SEQUENCE LISTING (54 sequences)

SEQ ID NO: 1 - DNA, 3420 bp, Homo sapiens
  CDS (11)..(1612)
gggccgagccatgcaggcgtcgcgcgtggactacatcgctccctggtgggtcgtgtggctgcacagcgtcccgcacgtcggcctgcgcctgcagcccgtgaacagcaccttcagccccggcgacgagagttaccaggagtcgctgctgttcctggggctggtggccgccgtctgcctgggcctgaacctcatcttccttgtggcttacctggtctgtgcatgccactgccggcgggacgatgcggtgcagaccaagcagcaccactcctgctgcatcacctggacggccgtggtggccgggctcatctgctgtgctgcggtgggcgttggtttctatggaaacagcgagaccaacgatggggcgtaccagctgatgtactccttggacgatgccaaccacaccttctctgggatcgatgctctggtttccggaactacccagaagatgaaggtggacctagagcagcacctggcccggctcagtgagatctttgctgcccggggcgattacctgcagaccctgaagttcatacagcagatggcgggcagcattgttgttcagctctcaggactgcccgtgtggagggaggtcaccatggagctgaccaagctatccgaccagactggctacgtggagtactacaggtggctctcctacctcctgctctttatcctggacctggtcatctgcctcattgcctgcctgggactggccaagcgctccaagtgtctcctggcctcgatgctgtgctgtggggcactgagcctgctcctcagttgggcatccctggccgctgatggctctgcggcagtggccaccagtgacttctgtgtggctcctgacaccttcatcctgaacgtcacggagggccagatcagcacagaggtgactcgctactacctgtattgcagccagagtggaagcagccccttccagcagaccctgaccaccttccagcgcgcacttaccaccatgcagatccaggtcgcggggctgctgcagtttgccgtgcccctcttctccactgcagaggaagacctgcttgcaatccagctcctgctgaactcctcagagtccagccttcaccagctgaccgccatggtggactgccgagggctgcacaaggattatctggacgctcttgctggcatctgctacgacggcctccagggcttgctgtaccttggcctcttctccttcctggccgccctcgccttctccaccatgatctgtgcagggccaagggcctggaagcacttcaccaccagaaacagagactacgatgacattgatgatgatgacccctttaacccccaagcctggcgcatggcggctcacagtcccccgaggggacagcttcacagcttctgcagctacagcagtggcctgggaagtcagaccagcctgcagcccccggcccagaccatctccaacgcccctgtctccgagtacatgaaccaagccatgctctttggtaggaacccacgctacgagaacgtgccactaatcgggagagcctcccctccgcctacgtactctcccagcatgagagccacctacctgtctgtggcggatgagcacctgaggcactacgggaatcagtttccagcctaacagactttcgggggttcctgcctcctttttccgttctggtttttaattagtgcaaatacaagctgcgtttctttaatagaaaccaaaggcatctggagcccgagaggcctcctgctgggcagaggagcagctgggattcccgaccaaagccccagggggtgcagaagactcaccacgcgggccagcctctctcttttgccctgctctccacaccagaaatgcccccaggtgcttggctgcctcagaggtaccatccctgagctggctgcctggccctgctcacccctacgcctcgcccttgccaggaggggagtggcagtgaggagggggccaggtcaggcaccaccatcaagagagctgtgtgttctctctggtcccacaacgatgactctgcctcttgtcagcccagccaagagcccagacgacccctctgtcctcgttccctgtcctcgttccctgcaggtaacatgagaagggctgatcaggagatgctctttaagaagttcgcacccctgctgacaccagaacaagccaaatcagagttccagggccagacaggctcttcctgggccacagaggggaggcatcaggaaagctctgcagtggggggctggtggctccggggctgggggatcacaggctggtgaaccccggtgggaacagaggtgaaagcctgccacattccgcctgtctccctaaccctccattgcctcgcctctattccagaatcaatgctgcagaatgtgttagctgcagataggcatggtctcaggtatgaacagacactttgaaacgactttaggtctttcttttctccagtgttttaaacatgttgattatccaaagaattgaaactcctagcacatccagtttttacaacagatttgcagctcattccttaccctggttaggtcactacttttgcagattttgctggcactgatctggagatctgcagatctggaggagacgggaaggagtcgattcttaaataaggatcagtgaggcatcctgtcccaagctactgtttggtggggatctgggttcatctcacccacagagggaggatctttaagaggagaaaaaagccaagagggaaagccagagttccctgttctaggggactagccaaatgcctacatcagctgtcccctccctgttgtctccaagtaagtttgccagaaaaggttttagcaaagtgctacaactgtgtctttataggaggataggcctctgccctgccccacccccaccacctgtccccacccagtgtcccaggccacaggagcttattggccaggagggaataatgtcccccaatactgcctgttgagggaccagagttggggtctttggtgcttccaacctcctgccaacctggagttcacaacaccagagccccacggcctcgcacactgaagcaggggcgtgcggtgactcggtgcttctgttttggaagaaccacctgtcatcaaaacatggacagcagggtgttctcagctcccagcgaagcctccacaacagaatggggccacagggcagccgggactccctgtctcacctacattaacccatgcatactgtatgccataaactcactttggtatatccgcgtcacatgcagagaggaactctgcgacgtcaaagtgttgcttcttaaagtttcattattggcaactagagggttgtttttaatgcatggaaactaaacagattcctcggggagttcctgaaggaaccaggtgggcaaacctttgcttatatacatgcggcctcacctggaagagaaataaaccacttgtact

SEQ ID NO: 2 - protein, 534 aa, Homo sapiens
MQASRVDYIAPWWVVWLHSVPHVGLRLQPVNSTFSPGDESYQESLLFLGLVAAVCLGLNLIFLVAYLVCACHCRRDDAVQTKQHHSCCITWTAVVAGLICCAAVGVGFYGNSETNDGAYQLMYSLDDANHTFSGIDALVSGTTQKMKVDLEQHLARLSEIFAARGDYLQTLKFIQQMAGSIVVQLSGLPVWREVTMELTKLSDQTGYVEYYRWLSYLLLFILDLVICLIACLGLAKRSKCLLASMLCCGALSLLLSWASLAADGSAAVATSDFCVAPDTFILNVTEGQISTEVTRYYLYCSQSGSSPFQQTLTTFQRALTTMQIQVAGLLQFAVPLFSTAEEDLLAIQLLLNSSESSLHQLTAMVDCRGLHKDYLDALAGICYDGLQGLLYLGLFSFLAALAFSTMICAGPRAWKHFTTRNRDYDDIDDDDPFNPQAWRMAAHSPPRGQLHSFCSYSSGLGSQTSLQPPAQTISNAPVSEYMNQAMLFGRNPRYENVPLIGRASPPPTYSPSMRATYLSVADEHLRHYGNQFPA

SEQ ID NO: 3 - DNA, 1605 bp, Homo sapiens
atgcaggcgtcgcgcgtggactacatcgctccctggtgggtcgtgtggctgcacagcgtcccgcacgtcggcctgcgcctgcagcccgtgaacagcaccttcagccccggcgacgagagttaccaggagtcgctgctgttcctggggctggtggccgccgtctgcctgggcctgaacctcatcttccttgtggcttacctggtctgtgcatgccactgccggcgggacgatgcggtgcagaccaagcagcaccactcctgctgcatcacctggacggccgtggtggccgggctcatctgctgtgctgcggtgggcgttggtttctatggaaacagcgagaccaacgatggggcgtaccagctgatgtactccttggacgatgccaaccacaccttctctgggatcgatgctctggtttccggaactacccagaagatgaaggtggacctagagcagcacctggcccggctcagtgagatctttgctgcccggggcgattacctgcagaccctgaagttcatacagcagatggcgggcagcattgttgttcagctctcaggactgcccgtgtggagggaggtcaccatggagctgaccaagctatccgaccagactggctacgtggagtactacaggtggctctcctacctcctgctctttatcctggacctggtcatctgcctcattgcctgcctgggactggccaagcgctccaagtgtctcctggcctcgatgctgtgctgtggggcactgagcctgctcctcagttgggcatccctggccgctgatggctctgcggcagtggccaccagtgacttctgtgtggctcctgacaccttcatcctgaacgtcacggagggccagatcagcacagaggtgactcgctactacctgtattgcagccagagtggaagcagccccttccagcagaccctgaccaccttccagcgcgcacttaccaccatgcagatccaggtcgcggggctgctgcagtttgccgtgcccctcttctccactgcagaggaagacctgcttgcaatccagctcctgctgaactcctcagagtccagccttcaccagctgaccgccatggtggactgccgagggctgcacaaggattatctggacgctcttgctggcatctgctacgacggcctccagggcttgctgtaccttggcctcttctccttcctggccgccctcgccttctccaccatgatctgtgcagggccaagggcctggaagcacttcaccaccagaaacagagactacgatgacattgatgatgatgacccctttaacccccaagcctggcgcatggcggctcacagtcccccgaggggacagcttcacagcttctgcagctacagcagtggcctgggaagtcagaccagcctgcagcccccggcccagaccatctccaacgcccctgtctccgagtacatgaaccaagccatgctctttggtaggaacccacgctacgagaacgtgccactaatcgggagagcctcccctccgcctacgtactctcccagcatgagagccacctacctgtctgtggcggatgagcacctgaggcactacgggaatcagtttccagcctaa

SEQ ID NO: 4 - DNA, 47999 bp, Human
  mRNA (1936)..(2074): Exon 1
ctgtaaaagcactacagaagagccactttccccagcaactgcagggactgagtcagagacaagaccttttaaaaggaggttttcacccaaagaagttccctcccctgccgcagtctccaagctgatcgcctggctcctgcagttctccggctccctgtcctgttctggaacttgcttgcttccccttccactggccttagtccctgatctcacagggtcctgttccttctgcacagaggcctcccccagcacccccctctttgactcccgatcatccttcagctcatgtcactgtcctttgggaaacaaccctgacgcccaccttccatcccctctccctacagcagggactctgacatttcacaaggcctgcaatcgctgttttgcaggatacaagtccacctcacctcgcctactaggctgtagacgccatgcaggcgggaacgtgtgttttatccattactgtatcctctagaacagagaacgtgctcaagaaaatttgttgaaagacaccccaatgagagaagaatggctgtttccaagcttccgggcccctcagctttgggctcctgcagggccccagaaccgtgggatcacggccacgaggagagagcccgaggccaggacttcctctaatggacacagggcctttgagcaccgggtgactggggccccactccccagcagccatatgtgtttgcgagcgttttcagtcggtccgcatccacccggtcccttcagccagaaccctaggagccccgccccgtcctcccggggctcctttctcacccgcgtccctgccgcggtggatacgcacccctcaggctggctgcccagcggtttccacgcccaatctgccgcgaccccatcactccaccgcacctacccctgactcctgcttgtcctgcagcctgggctcctccctccgctggccaagtcacgccaagcagtttgcgattacaggacgggtgggggcctctgcatatgtgcaagtacggagggcccagggacgcgaggctccccgatggcaacctcccctgcctttgcttttccccagagccccctcctggcctgcgttcggcatcttctcgggtcgtcatcgccctctgcggtgcctgcgctcggggtgagggtttttgtcggcttttgttacatcaccgatgtaacagtgcctggacgtagtaggtgctcaataaatatgttgttgaatgcgcttcagtctccccgaatgcttgtcccttccgggcgctctcatcgctctcctaccttccccgctacacagctcgaaggtcccctcctccgggaagctcaccctgtcccacttgccaggaagaatcaagcaaattttctgagtgagacaccttgtcctcactcaggcgtcctgcaggcggcttcagcgagttgttggggcagatgcgccgggagctcccgagggcagggacggcgtcttatttctctttttaagtccacagggcttgcacaatagatgcccaaaaaatgagagtgaatttagaatgagtgaccgagtggctgaagattgcctccttcctttcccagtctgtcggttacttaccctgtgacaagcccttgggacgggcagtcgcctctctggcctatgactgctggctgcccccactgacaccttatacccttgatctgatcataaataagaagcagcccggggacctcgagactgaaactgatttagagacttctgggagcttttttcacttccctcctcccatccggccaggacagatccccgcccagaaaggggctgttgactgcagcgggctggcgcccagaactgggactgggatcgcggcggggcggggcgggcgcgggcgacgggagaggcggggccgtgtggcggggcgggcacggggcccggccgggaggtgagcgcactgttcgttcagcttgtgggtagcactcgggccgagccatgcaggcgtcgcgcgtggactacatcgctccctggtgggtcgtgtggctgcacagcgtcccgcacgtcggcctgcgcctgcagcccgtgaacagcaccttcagccccggcgacgagagttaccaggaggtgagtttacgccgccccagaccgcagccacgccgcgccgaagtccccgcactaccccctctcccctcgagagcctgcactttcccacgtgcctctcaaaacccttctctccccgcgccccctttcccgtcgtccctcgtcccgtttcccccaccctcccaggccccattccgccagtccctcctggtcgggaaccttcggggccggccaacctgaagcgactcgacttgaaccccccctgggcacagggcccagcaaggcccacaccccctccgcgctcccaggagtcacacccttaacggccaagctccccaagttaagcgctgaatgtctggctagggcagtacgtcccgtttcccggcctgtccctactctgctgaccttggcaggatctgcaccctacaaacagggccaggctccccagacctccctgcgcagataaacaaagctgcagaatctcccagcccgtctcaggtctggtcacgcctgctctggccccggagcccacctgtatcagttccttgtgttgggtcaaagattctggggccagtggaccaggatgaggtccatttattcagagccaggcgcccatctcaggactttggcccccggcggcctctccctgtgtcaagttctccagatatgggacagatcagggcaaagcagggcctcttggagggaatcctgatccagtgtgaggtgcccaagcgccagtgacagggagtctaataataacccctgacgacccggctgccatcaggcccaggcagatggtgcctgccctaccagagggaggggagcatcaggaccccttcctggggttcagtgggcactcaggagcaagacggcagtcgtggacccacccccctcccccagagcccagcctgtgggagtctgtgttccctgagaatgtggctgcctggggatagctccagggaatcttcctctcccagagaatgcatgaaaagaggcggatatgatttcagaaacagccgtgtgtgtgtgtgtgtgtgtgtgtgtgtgtgtgtgtccccatcccaaaccctgtgccccacatcccacacccggggtctccagccatagttttctcctctccactggaggaaatctaaacttttcattgggtcaagaagacccctaggtgggggtagggatggtaaggggactccctcaccctgatggccccagagacagttcctatcagcccaacagctgtggctggtggatcctgggcctgcccacagccccctcagtcccatgggcaggtggcagttgagccagatgaacctacctccagcctctgcccctcctcccaggattctggccccggctcactttgtgctgggcatcaaatggttccagagggtgtcccttcccatcggccactgctcctgggcagctgacaccagccctgcccactggctcctggtcccgctcaccccctcaccaccactcagatcgctgagtaggagatgagcgtggtgggccaggaccggaagtctggctctgggtgttatttaaggtggctcctgtttttggtaagttcccctgttgtgaccacaggtctctcctggatgtcttctttcctcctgagcaccagtcactaacagagtcaggtggaccgtcggtcatcccagtcttcagcaagcttgactggagcaagtgctatgccaggcgtggggtgaccgtggtaaccaaggcagcaggtcttcaggagaaattgctggggattttctgcggctgggctctgggcgcagtgctatggcagacaccaagcagtttgaggtggggctctgccctccagttggctggggtggattcccagttctttccatgtgtgaccacgggtaggtgtcttaacctctctgagccttgttcccttcatctgcaagaattagatgagatattccacatatcatgcctagtacagagagtcccatagcacataacaggagccagtgacacagatcattcaatggagtggctgagaggttcaggagtgtgtctcaggacagtaccctgctggagaatggagaggaaggagatctcttccacagactctgattgggggagactgaacaaaacgcctgcagaaagggcttcagctcatgccccacatgttcaaatgcacagtgctggctgtgtggggtgtggggggtgggtgcgtgtgcatcagagactaagcaaggatggaaacaggcaggtgctggccttaggatgtgggtggtgcgggggatctcaaaggcaagggggttcttggaagtgcagagatggtgaagagcaggtcacctggtgagctgggatttcctgagcaagttcagtgttccagaaccttcctgaggaagtaggggctctcactcactttctggggtgtttgggagccgtgaaagcccccagtcctcctgatagttgatcctcacccctctccccccagtgaacttgagcccaagtcctcgcaaaggccacttggcctgtgtcaccagccttgtggggaggctaccctccagtgagtagaggcatgagaggtgagcaccaaggcagggctgtggacgagggcaccttggcctggcgggcacccagctctgggctgccagatgtggctgtatgtctggaaggaaatcagcctagacctgcagcccaagtcttctcacctttgacagatgagggggattggatttggagacgattcctactctaaagagatgataaaagaactaatgagttcatggattatgaagcctgtgcgcagtgcctggcacactggaaactgctacaatcactgcttaggaaggagaagactgcagccagccggtgtccactgggcagtgttggggagaagggaatgggcacctggcactgaatccagcaatgtctctctgagcaaatgaacgaggcacttcggtgtccctgcctgagcgtgagtgccaccatcaccatccagccagcccctcaggggagaccagacactgatgagctcagttcttgaaacatcttagtaagctcccaaaggctgtgcaaatgttagttattgctactgtcaacaggcaggtggccacatgtgtgcttcctgttccagcttcccttgcccaggatcagccgaccagctgctctttggggacatgtgttaggaggcagaggagatgttgttaattttttttgtatttaggcatgtatttccacgtgttctctcattggttggttccgttcatagattcgccaaagtacaaagtggagggtacacagttggggactggaccggaactacaggacttcctgcttctgacctgggggcaggtctgaggaagaaggaccaggcagcaagcccatcccagccgagatctgagaagagctgaggttgggatgaggaagccccattcatccagtgtgaaattgagtcacggtcatcagacagtcatgccgcgcctgggagagggcttaatctttcccagtgggcctgggagggagactctccctctaggtgctcctagtttccctctcccagcctccccctgagaccggcacacctctctctatgtcttctggggccagggctgttgccccctcccctgcccagtgcagaagccccttgggtcccgctgccatggtcactgcggcccccaacttgggtcccagcttggcactctccgctctgggtttttcatgacacagtgacaaatcagaaataattgaagtccctttgatgaggggagaggcagagtctgagcaccagtgtgggagctgggggcctggtgttgctgtgggacaaggaggtggctgcagggcacagtgggaggcgtggggaggcgtgggaaattgtcttggcaaagctctgtccatctgagcaggggggcctggaagcagaactcttggctccctcctccattcccctccacctccatgcacatggggacatgcagtgttaattaggagcaagaaaataattgcccattaggagcagcaggtgccaccaagctggccctggaagagggtgctgctggttagggggaggggggtgggctcctacctgctcctcttttccctcctccttcctcctcccttgcccaccttccctcttctttcttcttcccttcccaattccttttccccctacaccctctcaccctgtcccacccaccacccccagttttttcccagcacctaggtagggttcccaggagggtttgggaagtaactccctaacaaaaggcttccaaaggctgggcagggtggctcatgcctgtaatcccagcagtttgggaggccaaggaaggaggatcacatgagcccaggagttgaagactagcctggccaacatagtgagagcctgtctgtatttttttttaaaaaaaggaaatcaaaaaaaaaaaaaaatgcctccgagtttgggtttctcaggcctcgctttcactggagctggacagcattatcagaaacctaattgaggccagctgcccagcttggagaggccccaggccacaggggctcagcggctgccgaaggggagaccgggaggctggaggaggcttgagagagtgaaacgggggagcaggctacatcacccacagcctgatccttctcggacgagccttccttgctgggtggggacattggtagattcaggagcttttcccactggggataaaatgatagtgggactgttcaggaggcaaactcaaagggtagctgggggcaggctggatggtaactgggcttcaggctacacaggtttgtctggggctcccttacatttttgtgttattaggaataacttggttgggcgtcgtggctcacgcccgtaatcccagcactttggggagccgaggcaggtggatcacttgaggtcaggagttcgagaccagcctggtccttttgtagtaaaagtacaaaaaaattagctgagcatggtggcacatgcctgtaatcccagctactcaggaggctgaggcaggagaatcacttgaacctgggaggcggaggttatagtcagccgagatcacgctgctgcactccagcctggtgagagagcaagactccgtctcaaaaaaaaaaaaagtacaaaaaaattagctgggcctggtagcacatgcctgtaatcccagatactcaggaggctgaggcaggagaatcccttgcacctgggaggcggaggttgcagtgagccgagattgtaccactgcactccagcctggcgacagagcaagactccgtgtcaaaaaaaaaaaaaaaagaataacttacaggaaagagtgcagtcttaagtgtgcagtgcagttcaatgaattgataccctcgagcacctgcgtgcgtggtatgtgtgtgtggagcaacaccatcccaatcagatcgtattcctgaccccctagaagcccccttcgtgctctcttgaaatccgcaccctcccaaaggtagcaatgattcggtgattcacatttctctggctactcatgagtatctctgactttgaacttcatataaattaatccatacagttctctgctgttccatgtctggctttctctctcaacctgacttctgggaaattcatcccagaaataccgtcatgtgtggctggtttccttttcatggctgtatagtattctaagactataacacatgttctatatcttttctcttgttggtggacgtttgtgtctttttgggtgtaggctgtgactgctgtgtgaagctgctgtgaacatttgtgtatcttctgagggacacagccctcctttctgtaaagcagaattgctgggtcatggcatgcatgttacacaatgatttgcaatttttttttttttttggcttggttggggttttttttgggaggttgggttactttttttttaagaaatgggatctcagcatgttgcctagcccaggctagactcaaactcctgggctcaagtgatcctcccatcccagcatccccagtagctgggattgcaagtgcatgccaccaggcctcgctggtttgcaaatgattaagaggatccctgggctttagcagagaggcctcaggggctgccaaggggcggcaggacagaacgtggcatttctatccactcctcactcagatggagctctgtgttgcagggctctgctaagcaagtcagtttgaagaaaaagttaagctactggaaaaagtttgagaacttctgttgacactaatccagtgctgcccagactttaatggaatatatgttgcctgggatcatctctagaggcaggtctgattcactagctccagggcggggctagagattctgcacctctaacaagctcccagaagatgtcagggccacaggtcacagggtggtaaactttacttcaaggaaagaggctctcaactcccagctgtcttggcctcctctgcctgggcctgggctgcagggccctctggtgaaggggcatggactgaggaccagaagaggggcagtgactttccaccatctctttctatccctgggcttgctggggtcagtcccggggacaggccatgctaatgagtggtccaggtggggagagccccagccccagccccctgccacactgtgaggtctttgggagagggaggtgcccagatctactgggccctggcttcccagctgccactttttttttttttctagaagggatctccctctgtcacccaggccggagtgcagtggtgcaatcatggttcactgcagcctcaacctcccagtctcaagcgatcttcccacctcagcctcctgagtagctgagactacaggcagatgccaccatgcccagctaattttttattttttatttttttgtagagacagggtctccctgtattgcccagactggtcctgaactcctgggctcgagcaatcctctcaccttggcctcccaaagtgctgggatataggcgtgagccaccacacctggcggaaagccacattttccaaagtcaaatccatctgttcccctcccaagtgagccatctctcctgcctttttctgggctgcccccatcacttcagctgagaagtctgtcagccaagagtgaccagcagagcagtccatgtcagaggcccgaagacctgcccgcatctcacagggctggcagcgtggcctccagcaggtgtctgtccccctggaaaatggccccaaagccccagactgcagagcccggccttggtttgtcaggtctatctctgtgtgtgcctgtccctgcacaagtctcgtctcccccttagaccgcaggtcctggcagtacccccttcctgctgattgccaccccctgcataggactccctgaaacaggctttccccagcagcacacccagagcactaggggttcgggactgggaacccctattgggggtgcatgggaagcaggggcggctgtcagctcaggagcctgacttagagccaggtccagccctggctggcggccgccaatcccggtgccatctggtgcctatctggcaaaatcaagagatgaatgagcagagagtggcaccaggcctcctgacagactggctcccattgcagggccccgaggtgctaggcaggcctcaggcccctccggccccctcagaattctggctttgctgctgcctctggcactggaggttcagctgtccctggtggggtgcagtggaagggactccagaagttgcatatcctgactatcccgccccctcacacatcccccaagcatggagctggcttctcgattctcaggatggaccccaacacctagaacaagctcttcacattctggtagaggatgatgcccagcctgggacagcggggtccttgcatggaagaggcaacagatacacagggctgtccttccaggccctgcctcagtggcactcagcgcccgaggcctctgggtactctggtcttggacggtatccctctccttgcctttttcccttggccatggagctcaccacccacagccctccctgctagctggggattcctgccttgtctcctgagtggctcggactcttcatcagcagggcctggtccgtatcttcatccagggtctgaaccagaaaccggagaccagctctggcacccctccctcactgaccacgagcccaccacttggcctcatgccctgtaagctccatcactcccacccaagtgcctcagcgccgggccctctgtttacagagtaggagcttgaaccctaggtggagcttggaaggatggacgagagatgcagctcaggcagtgggcactcaggcccaagggcaggcacttccagagccccgcactgcagggatgaagagtgacaacaggcctctgctctcttccagtcgctgctgttcctggggctggtggccgccgtctgcctgggcctgaacctcatcttccttgtggcttacctggtctgtgcatgccactgccggcgggacgatgcggtgcagaccaagcagcaccactcctgctgcatcacctggacggccgtggtggccgggctcatctgctggtgagtgtccctggacgctgggcttggggtgtgtgactcagtctgcaaggggccagggactgtttgaccatgttctgacggagctccagctaccttgatggaaaagcttgtccccagatgaatgttgtccctttttttttttttaccaagcatcaggaatcagatgcctggggtggtgatggggtctccagaaaggtctcccaagtggccccccacaaaccctgccccaatgtgggtttgtcctgtgcccagcagctgtgcaggcagcatttctgcaccgacactgggcatctccagtctctgtccctggcccctgccagctgctgccctgggtctgtgaggtcagaagaaagtttgactccagcacaaatcaattccatcttcttgtgagatcaatagcttttagtgccgttagccagttctttggtttggatgaggaagggagaatttcttttatttatttattttaaagatggggtctcactctgttacccaggctagagtgcagtggtgcgatcacagctcactgcagccacaaactcctaggcttaagcaatcctcccacctcagcctcccaagtggctgggactacaggtgcacaccaccatgcctgcctggctttttttttggggggcggggggtgggcggggaagagacaggctttcatcaagttccccaggctgatctcaaactgctgagctcaagtgatccacctgcctcagcctccaaaagtgctgggattacaggcgtgaggcactacacctgggctgagactatgttgctcaggctggtcttcaacttctggcctcagacattgctcccccctcagcttcccaaagcactgggattacaggcatgagccaccatgctgggccttaaagtgaggatttataactcccgtgcaggagtccatacccagcccaccacactgcctggccactccaccccagctggcgtccacatgcatgctggctgttttcaggagttgttgcgtttttcttgtttttaaatctataatgatttttccaaaagcctctagtgtccacgcagagacgcttctggggtgtggtcaccccagagagtagagtctcccactacctggcagcaggctggcagccaggcaggtcctggtcagcctgggggtgaggatgcctgggctctcatacccaggcagctcaccttgccactgcgagtttcttctctgttggaagatgttcttgatctcgtcctgcctgtagtgggaggctcacctaagccaaaacgcaggcgagacgctaaagcagccttgtggggcggacaaagctctgagcacaaggaaggggaccaaggctgctgcaatacatggccggggctgaaataagagactcagaggacagagttctggacaggccgggaggtcacagtcgagtgcggttcccagtacagtggaagagctttgagaagtgactggtgttaaaaatcctcctgcatggccgggcgcggtgactcatgcctgtaatcccagcactttgggaggccaggcgggtggatcgctgagctcaggagttcgagatcacccttggcaatgtagtgagacctcactctacaaatatgagccaggcatggtagtgcatgcttgtagtcctgactgaggcaggaggatgatgagcccagtgagcagaggctacagtgagccatgatcgctccatcgcactctagcctgggtgatgtgagaccctgtctcttacacacacacacacacacagatacacacatacatgcacacacacagatacccacacgacaaacttgttttggaagaatactttggaacagtaagaatggcatgaggagtcaattcttctaatatggaagaattatctgcagtgtagggaaaactcaaggtggaaggaatgtgcgtggcagaatccatcccacaacattctgggcgtgacttgtaccaggcaagagggcaccagcagtaagaaaacaggcaccaagtctgctttcatgatctgtcagcagaggctgagaactgtgtaccagtttgctttgaatcattgcaactaaagagagagggctgggcgtggtggctcacgctataatcccagcactttgggaggccaaggtgggtggatcacttgaggtcaggagttcgacaccagcctgaccaacatggtgaaaccctgtctctactaaaaatacaaaattagctgggcgtggtggcgagtgcctgtaacctcagctacttgggaggctgaggcaggagtatcatttgaacccgggaggcaggggttgcagtgagccgagattgcgccattgcattctagcctgggctacagagtcagactccgtctcaaaaaaaaaaaaaacaaccaaaaaactaaggagagagctctgaaggaaaggagcaggcatagaagtggatggcccacctgagtccaggcgttcctgggtgacatgggccccaaacccataggatgagaaggcgttaacttggcaaaggtgggggcagggcaggcggagaacctcccggcagagggaagaggaacgtgcaaaggcccagaggagggagcgagcatcggggccaagggaggccaatgttggcagaggcggaggggggcagttgaggggggtttggggccctctacaaaagggcttctcagagcacaatacaattggttcattttcttttaaataaagttacaaattggagggtcacgggaagacaattcatttaatgtataaatatctcagctgagcacttggtgaagctcctcccctctgtgtggatacagttgcattttctatgatggtgcatttaactgggttcaaagctagttacacaactctggttgaacccaatgaatattgacttatgcagtggatttctaaactgtagagggggtgtgtgtaagagagagacagagatacgtaaagcatgcaggaaccttgtttttttttttgttttttttttaatgtctcctgcccccccatcttggatgatacaggaacctcattttaaaaggcacattcccagaccacactgatggaatgggccgggctgcttatctgcacttaacaggctccccaggtgattctgttcccgtggtctgaggaccacatcctgaaaaacactaaattaggagttgagggcgccctggaaggaggtttcttttcttttctttcttttttttttttttttttgagatagagtcttgctctgtctcccaggctggagtgaaatggtgcaatcgttgctccctgcaacctcctcctcctgggttctagcgattctcctgcctcagtctcccgagtagctgggattacaggcatgcgccaccacgcccagctaatttttgcatttttagtagagatggggtttcgccatgttggccaggctggtcttgaactcctgacctcagatgatctgcctgcctcagcctcccaaagtgctaggattacaggcatgagccactgcgcctggtaggaggtttctatcacagtcctccagggggttccgtcctagatcctgtgttgtttaaagaacaacagaacaaatataagtgtggacgtgtacattcatctccccccaccccaacccaaatgagcattttggatgaaaactcaggaggctgcttaatcaatcttcagatggtctcaagtggtgcaggtcagatggaatatgatggatgacaagagtcaagatttgaaatgatttgttcatgctggaatgttggactaaaaccaactagatgagatttcacagggatagattgaaagttctgcttttggttaaaacaacacaaaaatcaatacagcactataggaaggaggagagactgggcttaacagcaattcttgggaagaaattcaggggctgtttgtagaccacaagatcagtggggggcggggggaaccagggcgggtgcaagttcaggtcagtgccctgggaagtggaggacctaaggcatgacccctgctgcgctcagagctgatgaacaacctggacttgagtgactcactcaggccacatttaagagtgacattaaccaattagactttcatttggaggacagtgatggcatgggcaagggtctgaaaccttagtcccaagaaggacacctgcccacttggagagcatgaggtgagagggttatgaggaagccaggttcagctgtctgatggggcacggtgcagaactggaaacttcttccaggctgccctagaagacaaatgaagatccagggggtgagtggtttaaggaggcaggtgtcagctcagtagaagaaggaacattctataagttgggagttcacagtggaagaacagactgtgacatgggtggcctgaggatgttgggggaaagggtggaggatgtcgagagagtccacaattcctggaggtgtcaagagagacccaaggttggcaatgagtatgtgatctcagtagacctcagcgattcccaggcccacctgtgtcagaatcccctgggggctggtaaaagtgcaggtaacctaggccctccccagatctactgcatcagcatcccttgggctggctacagtctcccaacttttggagtcttaggactagaggacagtattttcctattgcccaaataaaatgtcctgtagaaccagcctgccagggaccctgaaccttcacctccccacccccagccccacctgccagaatgtgagatgtcatccacctactcaccgcagcccccaggaaagagttcctcagctcaagtcaaaaacctgctctccatagatgttcacagcagctgtattcagaattgccagaacttggaagcaacgaagatgtctttaacaggtgaatggataagctgtggtccatccagacaatgaaatattattcagcactgaaaaaaatgagttttcaagctggaataagtcatggaagaatcttaaatccatattactaagcgaaagaagccagtccgaaaatgctccacactgtatcgtttcaactctatgacattctgaaaaaggcaaaactaaggagaaagtgaaaaggtcaagggttaaggccaggtgcagtggctcacgcctgtaatcccagcactttgggaggccaaggcaggtggatcacctgaggtcaggagttcaagaccagcctggccaacatggtgaaacctcctctctactaataatacaaaaattagccaggcgtggtggcggacacctgtaatctcagctactcaggaggctgaggcaggagaatcacttaaacccaggagtcgggggttacagtgagctgagatagcgccattgcactccagcctgggcaacagagcgaaactctgtctcaaaaaaataaaaataaaaataaaggggttagaggggagggggtacttaatgggcagagcacagaggaattttagggcagtgaaactaccctgtatgatatggtaatggtggacacgtcattttttcatttttttttttttaatttttgcagagatagggtcccactacgttgcgcaggctgtctccaactccgtgggctcaagcgacctatccgcctcagcctcccaaagtgttgggatcacaggcgtgcagccacacccagccaacacatgtcattatccatatgtccaaacctatagaatgtgcaacgctgaggaatgaaccctagtggaagctgtgggctctgggtgatgatgtgtgcatctgggctcatcagttgtaatggatgtaccactctggtgcagggttttagggtggagtaagaggctgtgcgtgtgtggggcagcagatatatgggaactctctactgtccacttaattttgctgtgaacctaaaactgctctaaaaacaagctttttgttttttttggagacagagtctagctctgtcacccaggagggagtgcagtgccatgatcttggctcactgcaacctccacctcctgggttcaagcaattctatgcctcaactcaacctcctgagtagctgggattacaggcatgagccaccgcacccgaccctaagcttattttttaatataagcaaacattctcctctgtgtatcacctctaacctctgtttgggatcttgcttatagtgctgcggtgggcgttggtttctatggaaacagcgagaccaacgatggggcgtaccagctgatgtactccttggacgatgccaaccacaccttctctgggatcgatgctctggtaaggctcccgggcagctggccgggtacagcacagcccacaaggtcagcgtggtcagagcaagggcccccgtcagatcccagctagctcagctgcacacggaggggctgtgtgacgcctgagggctggacccccgcctgcgctgcagttgcctctgctggggcaaatgggcaattgtgcgttccaagcctgcaggggtggcagggatgggggtgaggactaaaggaggcagctgtgggatcaaaccccattttccaacaagccgaacactttctgatttgtcaaaggagaccttgaacatggaaaatcagccccactgcattttttgttttcattttaagtgctgcatatggagtattgcaaggtccaggagatgccaagaggaaaaaactctcttcagagacaactgagggctgggcgtggtggctcactcctgtcatcccagcactttgagggaggccaaggcatgtggagctcagtagtttgagaccagcctgggcaacatggcaaaaccctgtctgtagcagcaatacccgtgttagccagttgtggtgtggttcagctacttgggaggctgacgcagggatatcacttgttcctgggaagcggacgttccagttagccaagatctcgccaccacgctccattcctttgtagcctaatgtcactctatctctttaattataccgtggaacgattttcgtggagccccgttgatctacgcgttctcgcatactctcccctggctctctctgactccttcgctgtcctggctcgtttctcccccgtgctcccccccccccccaagcatgagatgatctctcaccaaataaaagatacaaatagacaaaaatcattatcattattatttttgaagacagagtttcgctttgttgcccagcctggagtgcagtggcacaatctcaacttactgcaacctctgcctcctgggttcaagcaattctcttgcctcagcctcccaagtagctgagattgcaggtgcactccacctcacctggctgatttttttttttttttttttaagtagagatggggtttgaccatgttgcccatgctggtcttgaactcctgagttcaggtaatccacccacctcagcctcccaaagtgctaggtttccaggcacgagccaccacatccaaatgagacagaaattatttttaaaagaaagaacaaaatagaaattctggagtcattaaccaaaataaaaatatagcagagaagctacaggatggcatatttgagcaggcagaagaatcactgaacgtgaagatgggtcaattgagattatgtagtctgaggaatagaaagaaaagagaacaaagaaaaacagagtctaaagacctatgggacaccatcaagcatgccaacatacacataatggaagtcacagaaggagaggagagagagaaagggtaggaaagaatatttgaataaataatggttgtaaatattccatatttgatttttaaaaaaataatctacacatccaagaaactcaatgaattccaagtgggataaactcaaagatatccatacctagaaacatcataatcaaagctgaagacagagactcttgaaagcagcaagagaaaaatgactcatcacatacacaggatcatcaataagatcaatagcttaagttgagtgtggtggctcacgcctgaaatcccagcactttgggaggccaaggtgggagaattgcctgaggccaggagttcaagaccagcctgggcaacatagcaagaccctatctctacaaaaattaaaaaataaaaaagtagctgagtgtggtgacgtgcacctgttgtcccaaccactcaggaggctgaggtgggaggattgcttgagcccaggagtttgaggctgcagtgagctatgatcacagcactgcgctccagcttgggtgacagagtgagaccttgtctctaaaaaaaaaagaaaagaaaaaaaaaaaagaaatgataagggaattaaaatggtacactagaaaatacgtggcacaaaagaaggcagttatggagaaagaacaaaaagatgtaagacatataggaaaaaaaagcaaaatggcagatgtaaactttatcagtaattaaattaatgtaaatagattaaactctccacttaaaaccagagatagatagaatagattaaaaggaaaaaaccacgatccaagctgggcgtggtggctcatgcctgtaatcccagcactttgggaggccgaggtgggcatatcgcttgaggccaggagttcgagatcagcctggccaacataggtaaaacctcatctctccaaaactacaaaaattagctgggcgtgtgcatgcacctgtaatccntgctnctngggaggctgaggcaggagnntcgcttgaacccaggaggcggaggttgcagtgagccgagatcncgccnctgcnctccagcctgggtgacagagtaagactctgtctcaaaaacaaaacaaaacaaaactatgattccttgtatgccagctacatgtaatctacaagagacatactttatttttttattttatttttatttttttgagatggagtctcgctctgtcacccaggctggagttcagtggcatgatctcgactcactgcaatctcagcttcccaggttcaagcgattctcgtgccttagcctcccaagtagctgggattacaggtgcgtgtcaccacacctggctacaagacatactttagattcaaagaggcaaacaggttaaaagtaaaaggatggggaaagatataccatgcaaatggtaaccaaagagggctggagtgcttatctcaaagacagactttgagatgaaaaatgctactagagacaaatgaagacatcatatactgataaaagggttaattcactaggaaaacttaacaattataagcatgtgcatgtacctaataacatagccttaacatacaggaagcaaaacctgacaaaattgaaggagaaatagacaattcaacaataataatggcagacatcagtatcctactttcaataatagacataattactacgtgaaagatcaacaaggaaatagaagtcttgaccaacactatagacaagacctcccagacaactatagaaaactccactggacaggccaggtgcggtggctcacgcctgttatcccagcactttgggaggccgaggcgggcggatcacgaggtcaggagatcgagaccatcctggctaacacggtgaaaccccatctctactaaaaaaaaatacaaaaaattagcccagcgtggtggcaggcacctgtagtcccagctactcggaaggctgaggcaggagaatggcgtgaacccaggaggcagagcttgcagtgggccaagatcgcgccactgcactccagcctgggcgaaagagcaagactccgtcccaaaaaaaaaaaaaggaaaactacaccggacaataagcagaatatagtcttctcaagtgctcatggaacattctccaggatagacaatatgctaggccataaaacaagtctcaataaattttaaaagactgaaatcaggccaggtgtggtggttcatcccagcactttgggaggccagccaaggcaggcggatcccttgagcccaggagtttaagaccagcctgggcagcatgatgaaaccttgtgactaaaaaaactacaaaaattagccgagtgtggtggcacatgcctgtaggctcagctactcgggaggctggggtggaagaattgctcgggcccaggaggtcgaggctgcaatgagcagtgattacaccactgcactccagcctgggcaacagagtaaaactctgtctcaaaaaaaaaaaaaaaaaaagaaaaagaaaatgattataagggaatactgtgaacaattaaatgtcaacaaactagacaacttgatgaaatggaaaagttcctagaaagacagaaactaccaaaagcaattaaagaagaaacagaaaatgtgaagaggatctataacaaataaacagactgaattgtaacttttaagcttccaataaagaaaagccagggaccagatggcttcactgatgagttctaccaaacatttcaagaattaatagcggccaggcatggtggctcacgccagtaatcccagtactttggaaggccaaggagggaggatcacttgaggtcaggaatttgagacaagtctggccaacatagtgaaaccctaactctactgaaaaaagacaaaattagccaggcgtggtggcatgagcctgtagtcccagctactcaggaggctgaggcaggagaatcacttgaatccgagaggtggaggctgcagtgagctgagactgcaccactgcactccggcctgggcgacagagcgagactccatctcaaaaaaaaaaaaaattaatatcaattcttcacaaacttccagaagggaatacttcccaactcattctataaggccagtattacccatatccgttagcatgtaggtttcccagaaacctctcccaaaatagggctccccaacgctctcatcatccctttatggcggcccctagtcaaatattctctgattttacttgaactatagttttttaaaactgtttattttgagctaattttagacttacagtagtaacaggagacactgctgaggtgtcagccaaacctgcctcacccgtgtctgcagttggtcagctatgggaggccctcaccccacttagtcttctgtggccgagcccaggctgccatgtgggctctgccctatagcagcaggattggctctgactatgaatctgggtcccggccaggtgtggtggccacacctgtaatcccagcactttgggaggcccaggtgggtgaatcacctgaggtcaggagtgcgagaccagcctggccaacatggtgaaaccctgtctctactaaaaataacaaaaaaattagccaggtgtggtggtgggtgcctgtaatctcagcgactcgggaggctgaggcaggagaatcacttgaacccgggaggcggaggttgcagtgagccgagatcacaccactgcactccagcctggcgacagagcgagactccatctcaaaaaaaaaaaaaaaacaaaaaagagaaagaaagaaatggatgaacgggctactctggcacactgcctagagggtagccctgctctgcaaggagcagtacctttaaatataaataaataaataatgtttttaactggatggccagccgtcaactttctatttctgtcttgcattggcagatgtttccttcagggtccactcaaccccaggcccttagggctcagccaagggacaggagcctgtctgcccacccagcaggacacacatccaggggaggatgtgaatacaaggagaaggcgccggtttcctcctcctgcctcatgtccaggctcctcagagacaggatcgcagctgtttttgtttttgcaaagtcccattgtcctgccttcactcagcgttcaggacccggtgtgctgcggctagtgccacaacaaagcttttctcttatagaacaattagaaatcctattgtggggatacaaggtcaaggtagcaacatagagtctgtttctctaaaagagaatttgtaagccaaagtgctgaaagttttaagagaatcttcaaagaataatggattattttgttggtttctccatctgggtgtctctgtatatagtaaggtatagtaatgaatgcatacgtttattccagccctttacagcaggggccacgtggtgggcacacaggatagcctccttagtagagagcaacagaggctccagagggcagggatgtgcccacactcccagcgccagaggctagacctggcctcagcctccaacacataatggtgtgctcctttggcgccagcattctctctctcggagcccagatcctgtttaaggccaggtcagggactgccccctggtttgagggacatgagggctgggtgggtagtggcggcctctgtcttcagtccttcctccaggggcacaggctcgagatcttcaggcctggggcagcatggtgtgttcattcatgccttaagcagccaggccagcttttttttttttttttcgacacagagtatcctcttgtcgcccaggctggagtgcaatggtgcgatcttgactcattgcagactccgcctcccgggtccaagtgattgtcctgcctcaacctcccaagtagctaggactataggtacatgccaccacacctggctaatttttatatatttttttgtagagacagggtctcactgtgttgcccaggctggtctcaaactcctgggttcaagcaatccgcctgcctcagcctcccgaagtgctgggatgacaggcatgagcgtaattatcgcacctggccaggccaccttctgctgcctgcaaagacctcttccgggatgagaatcagaagctctacctccatggcttttgccccctgctcctccctaggtttccggaactacccagaagatgaaggtggacctagagcagcacctggcccggctcagtgagatctttgctgcccggggcgattacctgcagaccctgaagttcatacagcagatggcgggcagcattgttgttcagctctcaggactgcccgtgtggagggaggtcaccatggagctgaccaagctatccgaccagactggctacgtggagtactacaggtgaaggaccggtgggaggcagagggaggggcagcagcggctacatcagctttgtttatccaaacctgcatgttgagctccaggtggagaagggccactggaaagtatttttttttgttttgtttttttaagacagagtcttgctctgttgcccaggctggagtgaagtggcacgatctcagctcactacaatctctacctccggggttcaagagattctcttgcctcagcctccctcccaagtagctgggactacaggtgtgtgccgccacgccagactaatttttgtatttttagtagagacagagtttcaccatgttggccaggctagtctctcgagctcctggcctcaagtgatccactggcctcagcctcccaaagtgctgggattacaggcatgagccaccatgcccagcccaccagaaagtcttgataagccattggccaaaatcaggagaacatgggggcttacaggggccacgtggtgagcatgcaggatagcctccttaatagataggcagcagaggctacagaggacagggatgttctcacactccccgcgccaaaggctggacctggcctcagcctcccgcacacggtggggtgcttctctgggactagcattcttcgtggctggtgcagggtggaaacgccacatgcttcataggttcctcgcctgccagagttcatgggacaggatttcttgtatccacagacacagccacagggttatggggggttgaggagatgagacccggctaagggtgctaagaagttgccatcaggggaggctctgacctacaagtcattgtgctcccagggcctgggcctaagcgtggaggaggcacaagggaggaatgaggctgtgtccccagactcactttttttttctttctttttaagacagtatctcactctgtcacccaggctggagtgcagtggtgtgatcatggctcattgtagccttgaactcctgggctcaagtgatcctcccgcctcagcctcctgagtagctgggactacaggcatacaaccacacctggctagtttaaaaaaatttttttttgcagctgggactgtgctcacacctaccagctctggggtcctccccacaccttgcctccacagcccgtccagtccctgcatctccgccatgtcctggatgctctggattgaagggaccttggccccactccccagcagatggatgctcatggcaaaagagcctccctccaagggacagaaggaaactgccagctctaaggactgtctgtgacctcctgtggccccaaaacaggggtgtctgatgattctcctgatgcatgggccattcattgcctgagccttcgaaatcttatgtcaggcaggggcaggacccattttaacgtcactcttctctgagcacacctagcagaggactgtgtatgtttaagggcctcatgtgggcattttgttaatgacactgatggaattaagtcagaatttacatatcaaaggcggcttattgttaaggcgatggtattaaaatagtggaaggatggtgtcaactgcacatcagctatttcagcgtgggtgattgacaggaccccctgtgagtccatccccaactccccactgcttattcgggttgaggcagaagaggtttggctcatgctgtaatcccagcactttgggaggctgaggtgggtggatcacctgaggccagtaattcgagaccagcctgggcaacatggagaaacctcatctctactaaaaatacaaaaattagctggacgtggtggcgcacctgagtagtcccagctacttgggaggctgaggcaggaaaattgcttgaacccaggaagcggaggctgcagtgagccgagattgcgccactgcactccagtcagggcaatagagtgagactctgtctcaaaaaaaaaaaaaaagcagaggtctagtccaaggttggcacctatggcgctgaacatcagtgatattctaccccttctcctgtctttgcttccatctgtggcttctcctctcacctgagctgctctgcaaaaaaagccctcccctgaagggcccctgtctttgcttttgggatagtccttgagcataaccataaggcaaagcaaatatttcctagcatttttatacaaggcggggcgaagtattggaggagataaaaagccaaggggctcctctctttgagaggctacggttgagggaagagggagccagggtgggagtggctctggcaggcagagccgagttcttggttaggaatacatcccccagtcctcgcccagctcctctgggaaaacacatccgcctagaggagatgtttccaggaacccctgacaacatgaggcgtggaggcagggaaaatacattgaatcacagccacagagagtgggttagaagcacccctaactggatttcctgctgctctgcagagcggaggcgagtggtgagaagaaatctttaagtttgaaacatgaactgggtatttcgagaggtgccctgtagctggctgtgtcccttgcaggaagcttgatgtagacaatgtggaatttctgggccaggctctccccaccaggggccttggctttctaatctgtactaatctgtagaatgggatccagctcacacctatgatcccagcactttgggaggctgaggcaggaggatctcttgagtccaggagtttgagaccagcctggacaacatagacagacccggtatttataatacgtgtgtgtgtgtgtgtgtgtgtgtgtgtgtgtgtgtgtgtgcgtgcgtgtaaaaataagaatgtgatccaaacactagatggtttgcgaggttccttccagctctgagctaagtctaaggaaactggcttcttggtggcagcaggctctgcccactccccaaagcccccggctccattccggggaaccccagagacctagtgtgtgctgccccctgctcgtgcagagacaaggactttgattagtccccttcttttggcagtaagcctcaattcggggagtcagaaagaacctcccttggctgccaggcctccagcttgctccctcccctctctccctcggagcctctttgccactttctgccagctccagggcactccctgcccacgctccaaactccgtcttcccctttatgatgtcagtgaagcagaaaaccattgctggagaggagaggtcagcgactttacttggctcttgcagcaagagaaataatgatccaaatacttccctctttggggacggggaagcccctgtctctgtgcccgcccctcctctgtccactctcccctggtgcccagcacagggtaaagggcatggctgtcatgtgagtacactgggccttgtgaggacaccgttggggacctgtgtggaagggaaaggacgagggcacttgtgtttgggggtcttctcagtgtccgttggcttaagatactttgattcttcagcctgttctctggagaagcaggagggcagggctgttgaagtccttggctgccttgccccatcaacctttatatttagcaagcacttaatccttcctctggccctgtgagatgaggggttccagctccctctacagatatggaaaccaaggtctcaaggggtgaagtggcttaccaaggtctcccagctggggtgtgactgcctttccccatcagtccagccctggggacaaaaaggccagaggaatgaagggcttggaggaggctggaggtctgcagggctgtaggtaccctgacctggactcccgggttccacactgagccctctgatggtcagaaacgagcccactcaccttgggtatcccttggtgccaagctcagtgcctggcaaaattgttgtttaaaattgaactgaagttttttgttgttgttgtttttgagatggagtctcactctgtgcncagctgggagtgcagtggcgtgatcttggctcactgcaacctctgtctcctgggttcaagtgattctcctgcatcagcctcccgagtagctgggactacaggtgcccgccaccacacctggctaatttttgtatattttgtagagatgaggtttcaccacgttggccaggctggtctcgaactcctggcctcaagtgatctgcccgtcttggcctcccaaagtgctgggattacaggcgtgagccactgcgcctgtcctgaacctaaaagtcttaagttatggatcccatccagcaagggggcaagatgggaggccttggggagccaggctgagtgtttgtcaaagaggcaggcaccttaggggctgtgttagatctgtggctataaacatcaggccttggctggtctgatgctgtctgagagttctagaagcaaagggaagcagggtcctaggcgatgcctgctgggaggccgcagaaagacacttcagagctgtgctgtccagggtggtagccccagccatgtgtggctattgaaattaattaaaattaagtaaagtttaaaatgtatttcttcagttgcacaagcatatttcaagttctcggtagccacctgtgggtagtggcaaagaagagagtggacagcagggatttagaacagctcctccactggggaaagttctagtgcacagtgctggttttgacactcagcctagaggtgaaggcagggggtgtgaattgacagctttgtccaggcagcagtgggggtgcagctcccgaggcttcagcccacgcatgctgggtgcttgctggctctgggcagcatgtccatagtagagggtccagtgtgtcccagggctgccgtgcagcctctcacgtggccagcaggtcagaagttccaccctgctgctcctgaccatccctgcccctctacccaggtggctctcctacctcctgctctttatcctggacctggtcatctgcctcattgcctgcctgggactggccaagcgctccaagtgtctcctggcctcgtgagtatccctacccgtggacctgggacaaagagctgggcaggatgccatcatcagagagagacgagggcctggccagctccagagtgtggggaaaatagccaggctgcctgaggccaccttctgccccgtcctcagcactcagcagaggagacagacagcagccaccaactcaccatctggtcaccaaacgagcaagcattaggctttcctccttctctgggcctgactctgttgagcagctaagaaacatgggtccgtgcatgaaagcctctggccgtcttagccaacactgcccccttagcctgtgctctcacgcctgggtctctttagcctcaaggacctgggctccatggtcctttcctagtagtgagcagggcccacgtcagggcagggactgaggctgcctttggatccattgcaagggtctgggggcagggtgggtggggtgatggctgagaggaaggggaccccgcctgccaacgttgtcgcctgcctctctcctaggatgctgtgctgtggggcactgagcctgctcctcagttgggcatccctggccgctgatggctctgcggcagtggtgagttgggggagggagtgggtggtgggtggtggttggtctgccaggacactctggtgtgtctccaagcagggccagcttccggtcccagctcctaacctagaatcccagaatctcagaaccaaagggacttgcgtcatctgaaccggcccctctcgttacagatggggaaacaggcccagagaaaggaaggggactgcccagggtcagagccaggataagacactcatgcttcatacccagagagaacccccggctgcccaggcatgcttaggcttacacgtgcttaggcttaggcgtgcctgggtgaccagggcgcttctctctgggtgtgaagaactgaccgggtctcctgagaatgggtaggggtccaagcctcagactggggaatccctcccgcatcagaagctgctgctttgccagctccccaggccctgggactgtagaatgtggtgtctgacttgccttccatcccaatgtcccaggactcccgcggtctgaagcacacatgctgtatgtttctggatgtgatgtctcaaaatggcttaaaaaaaaaaaagttgggggcttcctatgcgggtccacatgatctttaaaggacctaacactggcgctaagctgtgtgcatgaggctgttcattcactcagctgacattgtttgctgggtggcactcgatggtacaagttgcctggggttgcggaagccaagaggacgtgatgtttgctgttgacaaagttccagcctttggaagcaagactcataggcagatcactgcaaccccaggctttgatacagtggaggtgcagtgtgtgtgtgtgtgtgtgtgtgtgtgtgtgtgtttgtgtgtgtgtgttcatgcacatgcacgtgatagaagagaacacagtttaccaggggaattcaaggaacccctcactgattcaaagtccacagactaaccctgggacatgcatctgttctgccatagtgacctgaggcttgataggataagatgggggtggcttgtacgagaagctccctaaccacactcacacctcgtccagtgacttgggggctggcactagctctgggcgtggacacaatgccctcagggactctggcactgctgacttagcaccttaggcagaggcttgtgcagaagggagaagagggggcattttctgtctttgcctcctgccaccccagtgcagtgaccacggaccaaggaatggtcgagaaaaggcccaagtcatgtttgaaagcatgcaggagctgggacggctgtaaacagtgccactgctggccagggtgatgcaggcagctgctggcgctccagcctctcctctggggccagggccacgcacagggaagctgcagggagcacacggcgaactcctccagggccccatcccgctttctgggcagcctctgccttctggaggcagcggtggcttccagcttgccgctgtgagctgccctgtggtggccgcccctcccagtggggctcctccctctcaccaggcttcagtgttcacgaaaaccagaggccatttaatctcccagctgggcgccctggaatgcggggagctggggagaaaggccatttccctaattcctctagtcaggcccctgagtgcgagatggggagcaggcggtggagctgaatgggcttttgacgggctggagatgctgctgcattttaataggaagccgggtgaggaggagccaggagggcggggagggcggccggctgtcccggggttgttgtggagagcttttgtgtggcctcccaatgccaccctgctctctgaggcctgttttgacagccatggggccagaggattgacaaggcctcccttccccacccccgacccctacccctccctgtccctagaaaggactccatgaggacagagcactgccctggagatctgctctgggtgaggcagggagaggagggagcagcggagctccaagcttccaggccctgggccacccctcgtggtcagcactgcgagtcaggaggtgtctggggtgaggggctagagcaggcagagaggctgggaagccctggaagaaggaaggggaaggaaatgggttcagctgtgagggaggaggcaatggctgtcacaagtgctttggccaccgagcctggggacagagaacttcaggggattcctccagctaaggggcagccaggcctgcccctgagtcagagggtgggagtgtggcctctgcagccaccctgtctgggcccacatcctcccttcaccccttcccagcagtggaaacccaggaaagttcctgaggttttctgtgcctcagcttcctcctaggtgaacacagagaataacaggacttacctcaaagggctgttgctagggaaagatgagtgggttcatcctgggaggcacacagaaaatgtccaataaacagtggccaccactggcttcctgtgcattcatcatcatcatgctagggtcacgggggtcccttcagacccacacctccattctcaggagctgcctgcaggctcagccatcacctgggctggttactgggcagcctctcccacctgccccaactatgctcagtgacctccttcctgtcccctctcctgccatccctatcaccatggcatgagtaatgcacacgtgtattagtccattttcacactgctgataaagacatacccaagactgggcaatttacaaaagaaagaggtttgattggactcacagttccacatggctgaggaagcccacaatcatggtggaaggcaaggaggagcaagtcacgtcttacatggatggcagcaggcaaagagagaggacgtatgcagggaaactcctgtttttaaaaccatcagatctgccaggtgtggtggctcatgcctataatcccagcactttgggaggctgaggcaggaggatcacttgagcccaggagtttgagacaagcctgggcaacatagtgagaccctcaactctacaaaaaaaaaaaaattagtctgatgtggtagtgtctgcctgtggtcccagttactcagaaggctgaggtgggaggattgcttgagcctgggaggttgaggctgcagcaagctgtgttcatgccactgcactccagcctggatgacagagcaagacactgtctcataaaaagaaatgttagtgagaattgtgatcactctttgggagtggtgctgggcatgcaggaagcacgcctggaaactgttgttcatagtgcttgcaacctggtcagtctttacatattctggatgcaagttctttatcaaatagataatttgcagatattttatccaagtctgtggcttctctttttgttaacattgttggccgggcatggtggcgcatacctgtaatcccagctactctggaggctgaagcaggagaaccacttgaacgcaggaggcggaggttgcagtgagcagatatgccactccactccagcatgggtaacaaaatgagattccatctcaaaaaaaaagaaagaaaagaaaagaaagaatgaataagacctactatttgataggacaacaaggtgacaacagtcagcaataacttaattgtacatttaaaaataactaacaggccgggcgcggtggctcacgcctgtaatcccagcattttgggaggccaaggtgggtggatcatgaggtcaggagatcgagaccatcctggctaacacggtgaaaccccatctctactaaaaatacaaaaaattagccgggtgtggtggtgggcacctgtagtcccagctacttgggaggctgaggcagaagaatggcgtgaacccgggaggcggagcttgcagtgagctgagatcgtgccactgcactccagcctgggtgacagagggagactccgtctcaaaataaataaataaataaaaataactaacaaaggataaattcttgaggggatggataccccattctccatgatgtgactattaatatctagtatttgatagcacaacagggtgactatagtcaaaataatttaattatacatttaaaaatagctgaaagagtataactggattgtttgtaacacaaaggataaatgcttgaggggatggatggataccccatttcccatgattgtatattgtatattgcatgcctatagcaaaacatctcctgtgccccataaatatatacacctactatgtaaccacaaaaattaaaaattaaaaaaaaatagcaaaacctgctcacaggcaaacctgctcacgtaatcctgtttttgagatagagtcttgctctgtcatccaggctggagtgcagtggcacaatctcgactcactgcaacgtctaccttccaggtttaagcgattctcctacctcagcctcctgagtagctgtgattacaggcacgtgccaccatgcctggataatttttggtatttttagtagagatgggttttcgccatgttggccaggctggtcttgaactcctgacctcaagtgatccaccggcctcggccacccaaagtgctgggattataggcatgagccacaatgcccggcctaatctcttgggcgttgaaaggcctttggttgtaaatctaccaaggcaagctctccttcagtttatcttcctcccaccagcaccagccactcatccacatcttttgtcctaaggctctgtgcaaccccaggagccttgcacagatcctcaatctgagcccctggaaacttgctatcaaaggcacaatttctgaaagtgcaggtggctggcagacggaaatctctgctaattccatggtgtgatatggcaggtacacaggtcaaagcgccaaccactgaactcattgagaaagccctcagacccagtggagtctagtgatgtcatgggggagtcagaaaggtcaagggcaatatgtgggaatggcccagcccattgcatttttacaacttctaatcagcatcagtgagaaacccttccttgcgtggcctgtgaaccccgcctctgatgctgctggagcactttcatttcacttctcagcatcctgtgggtgtgtcaggtctcagaaagactaagtggccctgcctaggtcacgcagctggtgacgggaggcccagaacttgaaatcagggccgagcccagcccacttcccaattccctgctgtgtcccatgaccagtagcacgcgtcagaggctttcccaaaggcctccatgtcccgcctgttggggaagccctggctttctctctgtctgggattgactctcagctctgtcctggttggagagaagagatttccaggatggtacccagacagtggtggagccagagtctggcaaccaacgagccttttggacaattgggtggggagttgggtcattgggttggagaggaggggttcaagaagaggcccagggtctcaaatctggtcctggtgtttcatgcccagcatgtatcaacttagcaagttagtgagtgatgctctattgtggacccctgaggcagtggctcctgaaggaggacggggccaccggcagagcagggcactcagaggggtcccctgcccggggagcatgtgccagaggagggggacatccctaaggaacaggccagaggacctgagctctccctggctgctctggaaggcggaggcccccagtttatccagtccctagagcaccccaggccagatctggggtgcgcctggcttggaggccatgtccttgctgccgcctctcctggggcctgcgtctgctcccaccctgccgcatgggccttagcaatcacttagccaggccgtgctgtggggccaggtcctccagctcataagtggcagccgcctctcagctccatgcccttcccaccccatgtcttggcaggtgagcacctgcacccaaggctcacagatgcttcagcaccaacctcattctttagaaagagtttgggggaggcttcatagattgaacacccgtccctgtagcacttgatcatagcaagacaaaatgtaaaacagcagatgaggccatggtgtaacgaactcccacacacacagcctgcagctgccctggggcccaggatgctggcccccaaaggtctgtcccagaagaggcattcgggaataagagtaacagcatgcgctctgccccagtcacttttctgagcactttacttgcattgggtgaatagcattatcatctccatgtcacagatgaggcaaccggggcacaggaagaataagtaacttgtccaaggcaacagagctaggaaggggcagagccaggtttgaacccaggaggctggctccagactgtcctttgccacctgggagggagctcaaggggctggggtgatgggctcccggctcctcccagggccccgctgtcctgatacctgattttcctccaccccgtccctctctccctcactcaggccaccagtgacttctgtgtggctcctgacaccttcatcctgaacgtcacggagggccagatcagcacaggtaactacacactctcaggctgctgctgtggatgcataggtggccggactctgtgcccctacttacctctttcagccctgtagccatccaacccctcctcaaccctgaacttcaagtccagctcatgctctaggccatttcctagaaagtgcctcccttgggagctcagggacggggagggaggtctggcttggccaccaaggatgatagagatctcatctggtcatttgtgagctgcctaacgttatgccgttgccctgcagaggtgactcgctactacctgtattgcagccagagtggaagcagccccttccagcaggtatggccccccgaggcctgccctcaccctgcccacagccgctcccccttgaccctaagcctcaccactgaccaggatggcggaggtggcggggccttgcttgcctcactgtggtcctctccttagctgcttctgtgaggggggcgtgcttttggtcgtgggttctagattggtggttaacacatatgcagctcaatgcgggagctagttcccatgcagatgctgtccaggagctctggggtggggccgagattctgtactactcacaagctcccaggtgaggccagagctgctggtccaaagacacatctgagctgcaacgagggagaagactgtgcagctctcaagaagccagaggagtagggggagacttcctgcaagtggaggggcctgtgcagctgcagccaggtgggagggtgggaggggcaggagcataggcccctcagccactgtgggctcctgggagacggagcctcactgtggggctgtgtctttcctggctcagaccctgaccaccttccagcgcgcacttaccaccatgcagatccaggtcgcggggctgctgcagtttgccgtgcccctcttctccactgcagaggtaaggcagctgtgcaggaagaggggagccccagatgaaccctgacagccctttcccctgccccggccccagggccaggctgtgcacagcttgctgctggctctctgttctcccgcccccgtgactcggctccctcccttggctctagaggcccctgagcacagctgctgtggtggttttctccgccagctctcctccacagcccctcggcctctctctcttcccgcttcaggaagacctgcttgcaatccagctcctgctgaactcctcagagtccagccttcaccagctgactgccatggtggactgccgagggctgcacaaggtgcatggggaccctggggtcacgtggagagtgtgagggcacccagcaggccacaccttccagagaaaagccggtagaggccgaggggagcgatggcctgggtccccttccggcccaggaatggaactgaagccagataatgggaagggagggggtgcctggcttctccaggggctgagacagggagactcagtacaagggaccaggggttccggatgggagggttggatgtggttggctctgggtcactgggggaccctgaacaatgcaagggaggctgttccccattgtccagactcctggccatttcttcaacacatctgtctccctgggttgcaagtggatgtcccaatcccattgccagagtcttctttccaggaacttggaatatcagacagacacaatgatgcagggaacacatggggagcacgtgagagccccccacccaaaagcagtgagccagtgtatggcgacagctttcacatcccagatggatttagtgcagacagtctttgtcatcatcacagctgtcagctccatgtttggccctgttctgtgcattttatatcacagaacagtatgtcattcagttctcacagttatactatgaaccacacgttatttcctaggaaattgacttgcttgtgcgtgtgtatggatagatccatagatacagctatgtgtgtgtgcacgtgcgtgtgtctgtgtgtgtgcgtgtgtgtgcacttgtgtatatgtctgcggttgtgtgggtgcacatatgtgatgtgtgtatgtatgtgtgtggtatgtgcatgcttgtgcgcatgtgtgctgtgcatgcgtgtgtgtggtgcatgtgtatgtgcctgtatgtgtgtgtggggtgtgtggtgtgtgcgtgtgtgtggtgtgtgcatgtctgtgatgcgtgtggtgtgtgtgcatgtgtgttgtgtgcacatgtgtgcacatgtatgtggtgtgtgtggtgcatgtggggggtgcgtgtgtgtggggtgtgtgtgtgcgtgtatttataaagtggcttactgaagagtcccgagttggcacatcaggggcttgggtttagatcctggtagtctgacctcagagctaagaacatggcagtcacccctgtctcctctcttcttccccttctacatctaatccccagcaagtcttggcagctctcctctcagctgtaccctgaatccatccatttctctttctctccatggcttccaccacgatccggtcctgcccctccagcagacagtggcctcttccccactttctcttgccccctcaccacccatgagctttcacacatgcaaccagagcggtccttttgcaaactaggttggatcacatcccttccactcccacaccctccaatgtctttctgactccttttaaaagtccgtactcctctcccgcattgcaaggcccagcacgacccagcccctgctggcctatgttctctctctttcctgaacaggccaagcctgtcccctttgccccctcttctcaaggtgtgctgcttcttgccatcctggcctcagcttcagtgcccccgtctctggttccccaccctgcaacctcctccgccctgttcagagccagcttcttagtgcccagcacacagtaagtgctcagcaaatgtttccagcgtgaacccaagaagccccattgtgttaggctccagagtgtggacctttaatggtgctgcaaggcgctcgggatggggagcagccaggagaccccaggtccaggctcgggggagagggacttccagaggagagctcggcccgcaggctttgcccccgctccttcactagctgcatgtccctccttttcctccaggattatctggacgctcttgctggcatctgctacgacggcctccagggcttgctgtaccttggcctcttctccttcctggccgccctcgccttctccaccatgatctgtgcagggccaagggcctggaagcacttcaccaccaggtgggctgtctagtaaggaggggagcctcccacagcctggagttctgggagagcagcagacaggggcctctgctctacgatttagctgataacccaggactgaggagggcagcagagggcccgggcattcagcccaacaggctggctgacttctacagaagcttccccaagagtccacacagaccctccaaaggagttcggtggggcccagagcaagagctggggcccagggtggtgacagccctcaggccacagtctggcctcccttctcctctcttctcaccaggactttgcgatctggaatgaggagagttcttggtgctgggaggggtgccccaggaatagcaagaaggatacatggggtcactgttttcagggagcctgcagtccccttgaggagatgggcagggacacatggtgtacaggcacctggtgagcggattaggcaagaggacgcagggtagtggggacccacatgcgcatggtcagcgtggtggggagcctggaagagctggtcgtgaagctgggaatgggccagaatgatctgcaggaggcaggctggggaccgcgttttagaggagtctcaaggagacacgcgtggccagagagtcctgggaggaaaggcagggaagtaggacaggacctggctgcctcggagcctccttcacccttcacagccggccatcttctacccattgttctagaaacagagactacgatgacattgatgatgatgacccctttaacccccaagcctggcgcatggcggctcacagtcccccgaggggacagcttcacagcttctgcagctacagcagtggcctgggaagtcagaccagcctgcagcccccggcccagaccatctccaacgcccctgtctccgagtacatgaacggcctgcacacacacacaggttgggtagcactgcccggacagcatgttgggtggggtccacagtgccgggtgtggggaggaaggcatccaaggccacccgtggtccccactacagccacagggtgcctggagggaaggccnccggggagnatctagtcacccctccacacccacatgtgctgggggaagaaaccactgatgtcagcagcctctgccacaagctggtgggacaggtctcaggcccaaaaagctgcatttctccaggccatgctgctcccagcctccgttgcccatcagccacttgtccccaggcttcccaaggtgctcttctccatatctgcccactgcaccccctccactctatctgtggtttggttccatcccccacctcccatgtgctcacccatgggaccctcctgtagaaatctacacacacccccactcccacaccatcccctcctgagctctcctctcatccccgcaggaaccaagccatgctctttggtaggaacccacgctacgagaacgtgccactaatcgggagagcctcccctccgcctacggtaatatggctctggcccttccttgttggggtaatacataaagacaaaacctcctgccaacccagtgaatcctgtccacaaaggcagaaaagaatgaaactgttctgttattgaatatgcactaaaccagaccgtcctgggtatcccaggcagtccgctaaggagattgcaaagacgcaagaaatctcactccttcccctagccaggcaggaacaagacacagcccactgcatacctgtcatccagcaagaggacttgacggcactgtttgtcacagtagtccaccttaatccacttgctaattggggtggccatcctggttaagtaattgcctttatttatttatttttttttttttgaggcagaatctcactctattgcccaggctggggtgcagtggcgcaatatcggcttactgcaacccaggtttcaagcaattatcctgcctcagcctcccaagtaggtgggattacaggtgcgtgccacaacgccaggctaatttttgtatttttagtagagatggggtttctccatgatggccaggctggtctcaaacttctggcctcaggtgatccacccgcctcggcctcccaaagtgctgggattacaggcatgagccaccgcgcccggccagctaattgcctttatccagaggaaaaataaaactcctatttccgtgacaatcagatagctacaacttggagccaggcccctaagtgaactgtcactgagaaggagatgaggtgctgtcttcctgatgtttgtacttccaagagatggctcccagacctgaaaaaaccatacctggattgtaacactggcaagggccatattcagcctttaagaaggtttgcagacatctcaaaagacagaaattatttacaatgataagttttctaaaggaaatactgcaagaagagagggggtctctttccctttttgctccagagaaaattaaggaattttttttctttttagatttctgtttaccttaatccttggtctcttttgggctggacggtgggttggctgtggtggcgggtctgggagggctgaacccaaacacagccttcctcaatccgtctcagctccccatgtccctaggactgtgcccaagtagggccagtgcctcttgtccaccccaccacctccagccagagtacctgagatacagcctcaggggagtagctcagggtgggcaggttgagaaggaaagactatgctgtctcatcccagagaagtatccaaaggacacagaaatactggctgagacaaagaaagctgtaaacagacagtgaagtgaccgccaaggttcagggacacggactccagtgccaaaggcctgggttcaaatcccagctcatggcttatggcctctgtgactttggacaagcactttgctgctctgtgcctcagtttccccatctatagaatgagtatgggaatactagcacattcctcctcggtggtgtgtgcaatgggcaaattgattgttatcagagcgtacgtgcgaagcatccagtgagtgccgtaggacagtgagctgttatttgtgactttcttccaccccaccttctccacagagctgtggcatttatcaattttattttattttttattttttattttttttttgagacggagtctcactcttctgcccaggctggagtgcagtgatgtcatctcagctcactacaacctccacctcccaggttcaagtgattctcctgcctcagcctccctactagctgggattacaggcatgcaccaccacacctggctaatttttgtatttttagcagagacagggctggctggtcttgaactcctgacctcaggtgatccacccatcttggcctcccaaagtgctgggattacaggcatgagccaccgtgcccggccagagctgtggcatttataagaaaggacaaggggaatgaaaggccagaagtctgccatggggtgggcagaagggtcctggagccaggcatctcccttcattgggcctggcgaggcatttcagtgccccgacccagatagtttggaaggggacatccaacaggaaagcagaagcactggggcccgaatacctgggaccagcgtgatagacctgggccctgaggacccaggaagtgcacgcacaggcacccagggagtcagcttcccggtcatggcgcttggtgaaaatcatagtgtcagacccgagaaaagagcaagtggaacccagtccctgtcttcaggaagttccagtctatagaaggggcactcaagaaggctgcctgtagaaagtgctacaaggtactttgggaccagaaagaaggagccatctttgctgctgctctaggggattccagaaggttctatttaataggtggcgtttgagcacgaatgatagagcaggggctcacccaggggcagttttcccccctggatcaatttggcaatgtctggaggcattttcattaccacgcttggaggctggggatgggggctggatgctattgacatctagggagtggagcacccaccacaaagcacggtgcagccccaaatgcccataggttatggggcctgtcctctgcgtgtgggatgttcagcagcatccctggcctctacctgctagcaagcctgctggaaccaatccccctgtcattcactcctgtgccaccaccttggtgcttttgcctggagcacctcacctgctgtcccatcccacagctttacaaacacaagccccagcctcatgtcgttctttcttcctggggtccccgggagtgggctggttgaacctgtattcaaagtcagcaccatttattatttatgtttatttttttgagacagagtatcactctgtgacctaggctggagtacagtggtgcaatctcagctcactgcatcctccacctcctgagttcaagcaattctcatgcctcagcctcccaagtacttgggactataggcacgtgccatcacacccagctaatctttgcattttcagtagagacggggtttcgccatgctggccaggctagtctctaactcctggcctcaagtgatgcacctgccttggcctcccaaagtgctggggttacaggcatgagccaccatgcccagcctaaaagtcagcaccttttaaatgccagcctagcttctctcactaatcagaagcctcccgggttcctttttccaaccaccagctgccgtggccccaggaatgcagattgactttaatattaatctaagcacaacgtaattaggggaaatctgctgtgaaaaaagaattactctatgcactgtgatgccaaataaaaatgattttcagattcccttctgtttgaaagatccaggccactcatcctgccgttagcagagataatcagcaactgaccttatttgcaaggctcaaggacagaaaacccttcctccctagatttaagacctgcccagaaacacttattgctttggccaagttgactgcccagtcccacctagcccagcctgccctgggaagacccttccacctccatgcccggtaagggacctagagagacatctaatgatgccgcagaccaaggaaccccacagcatggagggtgtgggtgtctgccccagtaaagacagcacagctggctgcgccccaccctccccggggcgcctgctctaaacactccttagcaccgagcaagttcgagaggtggataaactcctccctttgccgagcaccctctctgggacagtcaggggacctcactgggtgggtgatgtcacgcctggtgatcgggatatacagcctgtcccttgctggaaatgtttcttcccccagctctgccttcaaggaataatctctcctgtccttttaggcactgtttcttggacttgatgaacaaaaatgacccagggcagttaaagatactgacattgagacttctcttcagacttacagaaacagaatatcttggggaaggagcctgggagattctaagtttaacaaacttattatcattcaagttgggaaatattgctttaagaaggagggtcttggccagcaccatgcctcatgcctgtaatcccagcactttgggagccaaggcaggtggatcacttgaggccaggagctcgagaccagcctggtcaacatggtgaaaccccgtctctactaaaaatacaaaaattagctgggcatggtggtgtgtgcctgtaatcccacctactcgggaggctgaggcaggagaatcgcttgaacccaggaggcagagactgcagtgagccaagatggcgccattgcactccagcctggatgacagagtgagattctgtctcaagaaaaaataaaaaagaagaaggagggtctcatacttgcgtatcatcagaatcacctgagggacccaccctgtttctgattcagaaaatctgagttggagcctggagatgtgccttttttgtttttttgagactgagtttcactcttgttgcccaggctggagtgcagtggcataatctcggctcactgcaacctccgctccccaggaccaagcgattctcctgcctcagcctcctgagtagctgagattacaggcccccaccaccacgcccagctgactttttgtatttttagtagagacagggtttcaccatgttggccaggctggtcttgaactcctgagctctggtgatccacctgtctcggcttccaaagtgctgggattaaaggtgtgcaccacgtgcctggccggagatgtgcgtgttgacatattctaagtaagctgctgctctcccctggcaacccgctttgcaacccatggttcttaagccaacttctcaactctcgtccccataattggaaccgtacttccccgcggggacctcccgtcaaaatctacgcccccctcacgccatccgcgcgccacccccttcccacggccacgccgccccaacgtcccaattgttccaacaaaaatatttatatctagtggatgtggcctgggatcgggatgtttttaggctccccaggtgtttctggcgagcagcccagtttgagagcagctgtgttattaaggctcttccctgctccatatcatttcgccctttcaacgtaagagctgggtgctcttatgtccattttgcaggtagggaaactgaggctagaaaaggctggagaacctaagccaaggtcacggggtaagatgcacagctgggatttgaacctgcttctctgagtctctgggcctctgccctcagcctgctggctgctgactgctcctccgtgcgctggtggaccctgacgctgcccccagtgtgaggggacaggctggatgctgggggtgtgtgctggggcggagtggacattggaggacagctggtctgtgtgtgcattttttatttgaggctgggaggcagaagtcttgatggatgtggggtggaggcacggccgggtttccctctgacacccgacccagttccactctgagaagtccccggcacctttgctggtgcagtgcttggctgactcagctctgaccatcccctctcttctctcttggcagtactctcccagcatgagagccacctacctgtctgtggcggatgagcacctgaggcactacgggaatcagtttccagcctaacagactttcgggggttcctgcctcctttttccgttctggtttttaattagtgcaaatacaagctgcgtttctttaatagaaaccaaaggcatctggagcccgagaggcctcctgctgggcagaggagcagctgggattcccgaccaaagccccagggggtgcagaagactcaccacgcgggccagcctctctcttttgccctgctctccacaccagaaatgcccccaggtgcttggctgcctcagaggtaccatccctgagctggctgcctggccctgctcacccctacgcctcgcccttgccaggaggggagtggcagtgaggagggggccaggtcaggcaccaccatcaagagagctgtgtgttctctctggtcccacaacgatgactctgcctcttgtcagcccagccaagagcccagacgacccctctgtcctcgttccctgtcctcgttccctgcaggtaacatgagaagggctgatcaggagatgctctttaagaagttcgcacccctgctgacaccagaacaagccaaatcagagttccagggccagacaggctcttcctgggccacagaggggaggcatcaggaaagctctgcagtggggggctggtggctccggggctgggggatcacaggctggtgaaccccggtgggaacagaggtgaaagcctgccacattccgcctgtctccctaaccctccattgcctcgcctctattccagaatcaatgctgcagaatgtgttagctgcagataggcatggtctcaggtatgaacagacactttgaaacgactttaggtctttcttttctccagtgttttaaacatgttgattatccaaagaattgaaactcctagcacatccagtttttacaacagatttgcagctcattccttaccctggttaggtcactacttttgcagattttgctggcactgatctggagatctgcagatctggaggagacgggaaggagtcgattcttaaataaggatcagtgaggcatcctgtcccaagctactgtttggtggggatctgggttcatctcacccacagagggaggatctttaagaggagaaaaaagccaagagggaaagccagagttccctgttctaggggactagccaaatgcctacatcagctgtcccctccctgttgtctccaagtaagtttgccagaaaaggttttagcaaagtgctacaactgtgtctttataggaggataggcctctgccctgccccacccccaccacctgtccccacccagtgtcccaggccacaggagcttattggccaggagggaataatgtcccccaatactgcctgttgagggaccagagttggggtctttggtgcttccaacctcctgccaacctggagttcacaacaccagagccccacggcctcgcacactgaagcaggggcgtgcggtgactcggtgcttctgttttggaagaaccacctgtcatcaaaacatggacagcagggtgttctcagctcccagcgaagcctccacaacagaatggggccacagggcagccgggactccctgtctcacctacattaacccatgcatactgtatgccataaactcactttggtatatccgcgtcacatgcagagaggaactctgcgacgtcaaagtgttgcttcttaaagtttcattattggcaactagagggttgtttttaatgcatggaaactaaacagattcctcggggagttcctgaaggaaccaggtgggcaaacctttgcttatatacatgcggcctcacctggaagagaaataaaccacttgtactaaaatgtgcgttccgtttctgacttccacagctgcaggaaataacgagggtattgaaagatggagcgctcccctccggcagggtcatgtggtgggaggcttagaagcagattccagtcgcgatttccgtgccaggactgaaagaggggcttgaacgaatgtcatggtgctcttgcgtggcttccagctgctcttacaggaccagggacaggacttatttctgccctgatctttctccagaattccccctaagggtcactgttctttttttgttctttttgttttgttttgttttgagacagagtctcgctctgtcacccaggctggagtgcagtggcgccatctcggctcactgcaacctccacctttggggttcaagagattctcctgcctcagcctcccaaggagctgggattacaggtgggtgccaccacacccggct

SEQ ID NO: 5 - protein, 534 aa, Human; identical to SEQ ID NO: 2

SEQ ID NO: 6 - DNA, 3408 bp, Mouse
  CDS (20)..(1615)
gctgcagcttgtgcaagccatgccggcggcgcgagtggagtacatcgcgccctggtgggtcgtgtggctgcacagcgtaccgcacctcggcctgcgcctgcaggacgagtacagcaccttcagccccggcgacgaaacttaccaggagtcgctgctcttcctgggggtgttggctgccattggcctgggcctgaatctcatcttcctcaccgtctacctggtgtgcacatgctgctgccggcgggaccacacggtgcagaccaagcagcaggaatcatgctgcgtgacctggacggcggtggtggctgggctcctctgctgtgctgcggttggcgttggtttctatggaaacagcgagaccaacgatgggatgcatcagctgatctactccctggacaacgcgaaccacaccttctctggaatggatgagctggtgtctgcaaacacccagaggatgaaggtagacctagaacagcacctggcccggctcagcgagatcattgctgcccggggtgactacatccagaccctgaagtttatgcaacagatggcaggcaatgtcgtcagccagctctcggggctgcccgtgtggagggaggtcaccacgcagctgaccaagctgtcccaccagactgcctatgtggaatactacaggtggctgtcctacctcctgcttttcatccttgacctggtcatctgccttgtcacctgcctgggactggccaggcggtccaagtgtctcctagcctccatgctgtgctgtggaatactgaccctgatcctcagctgggcttctctggctgctgatgctgctgcagcagtgggcaccagtgacttctgcatggctcctgacatctacatcctgaacaacacagggagccagatcaactcagaggtgacccggtactacctccattgcagtcagagcctaatcagcccgttccagcagtcactgaccaccttccagcgctcattgaccaccatgcagatccaggttggaggcctgctgcagtttgccgtgcccctcttccctacagcagagaaaagacttcttggcatccagcttctgctaaacaactccgagatcaggctgcaccagttgaccgccatgttggattgccgagggctgcacaaggactacctggacgccctcactggcatctgctatgatggcattgagggcctgctcttccttggtctcttctccctcttggctgccctggctttctccaccctgacctgtgccggacctcgtgcctggaaatacttcatcaacagggacagagattatgatgacatcgacgacgatgaccctttcaacccccaagctcggcgcatcgcggcccataaccccacgagggggcaactgcacagtttctgcagctacagcagcggccttggcagccagtgcagccttcagcctccctcccagaccatctccaatgccccagtctctgagtacatgaaccaggccatactctttggtgggaacccacgatacgaaaatgtgccactcatcgggagaggttcccctccgcccacatactctcccagcatgagacccacctacatgtccgtggcggatgaacacctgagacactacgagttcccgtcctaggggctttcagtgtacctgcctcttctgccagctcggttctcagcagatgcagaacccagctgtgtttctggaatagaaaccgaaggttcctggacacgaacaggctcctgtggctgcagagacacagctgggactcctgaccaaagccacaggtggatgtgaggcctgtgactggaccagcctctccggccttaactcccccaggactcagctgttgtccctgaggcagctgcccgcttcagcccaccttaagtcccaccctcagcagatgaaaaatggtaattgaggaggggaccaccagcctgagagcccttttctctaaaggcccttccattgggctcctgcctcttgttagcgtggccctaactaagtccaggccacaccctccagttacttggtcctcggtttctaggcggtggcaattggaggtgtgttaggagaacacctggtccaggatctacgttctgggaccacagggactctgcctagatgccagaaggacagcatcaagagatctccccagaggggatgggtggcttcaagctgggcactgcagcctggcagccatgggcacacgtataggagaaagcctgcaattctctaacctccaccagccaaccccattctggaaccaggtgtcagctgcaggtggggacagtctcaggtatgaacagacactttgaaagcacctgaacaggttcccagtgttttaaaacaggttgatgatccaaagactcacgcctcccgcaggcatggccaatacttgcaatagactcaggctcggttgggttttatacccgatgttctactgtggctctcacacaagccatcttacagatctggaggagaggccaaggagtgagctgtggggtttttggggttttgttttgctgcggtctccttatataccctgactggccctggcctagaactcacagatctgcctgcctctgtctgcagcagagtactgggctcaaaggcgtgtgctaccacaccacaccaggccaggagcagatttgctttgattttttttttttttttttttttgagacagggcttctctgtgtagccctggcttgtccttgaactcattctatagaccagactaacctcaaattcagagatcacctgcctctgccttgagagtgccaggattaaagggtgcgccatgatgccacatcctggtcttttggcttttttttttttttttaataagatatcagtgagacattctaccccacacggctagagaaggacatctggagccttgtaaagggaaagacatgatagctaagaaggccgagggacttcattctgaggaacagccacttctggccaggagaaaataataacccctgtcctgcctgccaagggacaggttaggggtctttggtgcttccaacctctgaccaacccagagtacactacacaccgaagccccgtggccttgtgcagccaagcgaggccctactgatggtgcgggacagggatgtgtccatttcagaagagccacattgtcattgtactggcagctcccagccaagccaccggcatcactgtgtctcatattaacctgtgcatactgtgagcgacctttgtatgcctgtgtcctgtgcagactcggagctttgacctcaggccttgctcctgatgtctcctctgcagcagctgaaggactttttaatgcatgtacattaaactaaaactcctccgggttctacaaaagtcgggtgggcaaacctcttcttggtccatgttcgccctcccccagaaataaaccacttccctta

SEQ ID NO: 7 - protein, 532 aa, Mouse
MPAARVEYIAPWWVVWLHSVPHLGLRLQDEYSTFSPGDETYQESLLFLGVLAAIGLGLNLIFLTVYLVCTCCCRRDHTVQTKQQESCCVTWTAVVAGLLCCAAVGVGFYGNSETNDGMHQLIYSLDNANHTFSGMDELVSANTQRMKVDLEQHLARLSEIIAARGDYIQTLKFMQQMAGNVVSQLSGLPVWREVTTQLTKLSHQTAYVEYYRWLSYLLLFILDLVICLVTCLGLARRSKCLLASMLCCGILTLILSWASLAADAAAAVGTSDFCMAPDIYILNNTGSQINSEVTRYYLHCSQSLISPFQQSLTTFQRSLTTMQIQVGGLLQFAVPLFPTAEKRLLGIQLLLNNSEIRLHQLTAMLDCRGLHKDYLDALTGICYDGIEGLLFLGLFSLLAALAFSTLTCAGPRAWKYFINRDRDYDDIDDDDPFNPQARRIAAHNPTRGQLHSFCSYSSGLGSQCSLQPPSQTISNAPVSEYMNQAILFGGNPRYENVPLIGRGSPPPTYSPSMRPTYMSVADEHLRHYEFPS

SEQ ID NO: 8 - DNA, 1599 bp, Mouse
atgccggcggcgcgagtggagtacatcgcgccctggtgggtcgtgtggctgcacagcgtaccgcacctcggcctgcgcctgcaggacgagtacagcaccttcagccccggcgacgaaacttaccaggagtcgctgctcttcctgggggtgttggctgccattggcctgggcctgaatctcatcttcctcaccgtctacctggtgtgcacatgctgctgccggcgggaccacacggtgcagaccaagcagcaggaatcatgctgcgtgacctggacggcggtggtggctgggctcctctgctgtgctgcggttggcgttggtttctatggaaacagcgagaccaacgatgggatgcatcagctgatctactccctggacaacgcgaaccacaccttctctggaatggatgagctggtgtctgcaaacacccagaggatgaaggtagacctagaacagcacctggcccggctcagcgagatcattgctgcccggggtgactacatccagaccctgaagtttatgcaacagatggcaggcaatgtcgtcagccagctctcggggctgcccgtgtggagggaggtcaccacgcagctgaccaagctgtcccaccagactgcctatgtggaatactacaggtggctgtcctacctcctgcttttcatccttgacctggtcatctgccttgtcacctgcctgggactggccaggcggtccaagtgtctcctagcctccatgctgtgctgtggaatactgaccctgatcctcagctgggcttctctggctgctgatgctgctgcagcagtgggcaccagtgacttctgcatggctcctgacatctacatcctgaacaacacagggagccagatcaactcagaggtgacccggtactacctccattgcagtcagagcctaatcagcccgttccagcagtcactgaccaccttccagcgctcattgaccaccatgcagatccaggttggaggcctgctgcagtttgccgtgcccctcttccctacagcagagaaaagacttcttggcatccagcttctgctaaacaactccgagatcaggctgcaccagttgaccgccatgttggattgccgagggctgcacaaggactacctggacgccctcactggcatctgctatgatggcattgagggcctgctcttccttggtctcttctccctcttggctgccctggctttctccaccctgacctgtgccggacctcgtgcctggaaatacttcatcaacagggacagagattatgatgacatcgacgacgatgaccctttcaacccccaagctcggcgcatcgcggcccataaccccacgagggggcaactgcacagtttctgcagctacagcagcggccttggcagccagtgcagccttcagcctccctcccagaccatctccaatgccccagtctctgagtacatgaaccaggccatactctttggtgggaacccacgatacgaaaatgtgccactcatcgggagaggttcccctccgcccacatactctcccagcatgagacccacctacatgtccgtggcggatgaacacctgagacactacgagttcccgtcctag

SEQ ID NO: 9 - DNA, 56 bp, Artificial Sequence
  Forward primer comprising a T7 RNA polymerase binding site
ggatcctaatacgactcactatagggagaccacatgccgagccatgcaggcgtcgc

SEQ ID NO: 10 - DNA, 25 bp, Artificial Sequence
  Reverse primer
ctgttaggctggaaactgattcccg

SEQ ID NO: 11 - DNA, 23 bp, Artificial Sequence
  First mentioned RT PCR primer on page 84
ggtgaggccgcatgtatataagc

SEQ ID NO: 12 - DNA, 22 bp, Artificial Sequence
  Second mentioned RT PCR primer on page 84
ggtatatccgcgtcacatgcag

SEQ ID NO: 13 - DNA, 22 bp, Artificial Sequence
  Intron A forward primer (Table 1)
ccgtgaacagcaccttcagccc

SEQ ID NO: 14 - DNA, 21 bp, Artificial Sequence
  Intron A reverse primer (Table 1)
ccagccccaggaacagcagcg

SEQ ID NO: 15 - DNA, 20 bp, Artificial Sequence
  Intron B forward primer (Table 1)
cctgctgcatcacctggacg

SEQ ID NO: 16 - DNA, 20 bp, Artificial Sequence
  Intron B reverse primer (Table 1)
aaaccaacgcccaccgcagc

SEQ ID NO: 17 - DNA, 20 bp, Artificial Sequence
  Intron C forward primer (Table 1)
ccacaccttctctgggatcg

SEQ ID NO: 18 - DNA, 20 bp, Artificial Sequence
  Intron C reverse primer (Table 1)
ccaggtgctgctctaggtcc

SEQ ID NO: 19 - DNA, 22 bp, Artificial Sequence
  Intron D forward primer (Table 1)
cccggctcagtgagatctttgc

SEQ ID NO: 20 - DNA, 20 bp, Artificial Sequence
  Intron D reverse primer (Table 1)
gagcaggaggtaggagagcc

SEQ ID NO: 21 - DNA, 20 bp, Artificial Sequence
  Intron F forward primer (Table 1)
cctcagttgggcatccctgg

SEQ ID NO: 22 - DNA, 20 bp, Artificial Sequence
  Intron F reverse primer (Table 1)
agccacacagaagtcactgg

SEQ ID NO: 23 - DNA, 22 bp, Artificial Sequence
  Intron K forward primer (Table 1)
ccttctccaccatgatctgtgc

SEQ ID NO: 24 - DNA, 22 bp, Artificial Sequence
  Intron K reverse primer (Table 1)
ccactgctgtagctgcagaagc

SEQ ID NO: 25 - DNA, 22 bp, Artificial Sequence
  Intron L forward primer (Table 1)
cctgtctccgagtacatgaacc

SEQ ID NO: 26 - DNA, 19 bp, Artificial Sequence
  Intron L reverse primer (Table 1)
cgtagcgtgggttcctacc

SEQ ID NO: 27 - DNA, 20 bp, Artificial Sequence
  Intron M forward primer (Table 1)
cactaatcgggagagcctcc

SEQ ID NO: 28 - DNA, 21 bp, Artificial Sequence
  Intron M reverse primer (Table 1)
cgtggtgagtcttctgcaccc

SEQ ID NO: 29 - DNA, 20 bp, Human
tctcttccagtcgctgctgt

SEQ ID NO: 30 - DNA, 20 bp, Human
ttgcttatagtgctgcggtg

SEQ ID NO: 31 - DNA, 20 bp, Human
tcctccctaggtttccggaa

SEQ ID NO: 32 - DNA, 20 bp, Human
ctctacccaggtggctctcc

SEQ ID NO: 33 - DNA, 20 bp, Human
tctctcctaggatgctgtgc

SEQ ID NO: 34 - DNA, 20 bp, Human
cctcactcaggccaccagtg

SEQ ID NO: 35 - DNA, 20 bp, Human
tgccctgcagaggtgactcg

SEQ ID NO: 36 - DNA, 20 bp, Human
cctggctcagaccctgacca

SEQ ID NO: 37 - DNA, 20 bp, Human
cccgcttcaggaagacctgc

SEQ ID NO: 38 - DNA, 20 bp, Human
tttcctccaggattatctgg

SEQ ID NO: 39 - DNA, 20 bp, Human
attgttctagaaacagagac

SEQ ID NO: 40 - DNA, 20 bp, Human
atccccgcaggaaccaagcc

SEQ ID NO: 41 - DNA, 20 bp, Human
ctcttggcagtactctccca

SEQ ID NO: 42 - DNA, 20 bp, Human
ttaccaggaggtgagtttac

SEQ ID NO: 43 - DNA, 20 bp, Human
tcatctgctggtgagtgtcc

SEQ ID NO: 44 - DNA, 20 bp, Human
cgatgctctggtaaggctcc

SEQ ID NO: 45 - DNA, 20 bp, Human
agtactacaggtgaaggacc

SEQ ID NO: 46 - DNA, 20 bp, Human
tcctggcctcgtgagtatcc

SEQ ID NO: 47 - DNA, 20 bp, Human
tgcggcagtggtgagttggg

SEQ ID NO: 48 - DNA, 20 bp, Human
atcagcacaggtaactacac

SEQ ID NO: 49 - DNA, 20 bp, Human
cttccagcaggtatggcccc

SEQ ID NO: 50 - DNA, 20 bp, Human
cactgcagaggtaaggcagc

SEQ ID NO: 51 - DNA, 20 bp, Human
gctgcacaaggtgcatgggg

SEQ ID NO: 52 - DNA, 20 bp, Human
tcaccaccaggtgggctgtc

SEQ ID NO: 53 - DNA, 20 bp, Human
ccgagtacatgaacggcctg

SEQ ID NO: 54 - DNA, 20 bp, Human
tccgcctacggtaatatggc

(19) United States
(12) Patent Application Publication
(10) Pub. No.: US 2005/0153286 A1
(43) Pub. Date: Jul. 14, 2005

(54) Polynucleotides and polypeptides linked to cancer and/or tumorigenesis

(76) Inventor: Judith Ann Clements, Queensland (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/477,720

(22) PCT Filed: May 14, 2002
(86) PCT No.: PCT/AU02/00591

(30) Foreign Application Priority Data
May 14, 2001 (AU) PR 4971

(51) Int. Cl. C12Q 1/68; G01N 33/574; C07H 21/04; C12N 9/64

(52) U.S. Cl. 435/6; 435/7.23; 435/69.3; 435/226; 435/320.1; 435/325; 536/23.2

(57) ABSTRACT

The present invention is directed to novel TTYH2 polynucleotides whose expression is modulated in cancers or tumours and especially in renal cell carcinoma. More particularly, the invention is directed to isolated TTYH2 polynucleotides and the TTYH2 polypeptides encoded thereby. The invention is further directed to methods for detecting the presence or diagnosing the risk of a cancer by detecting aberrant expression of a gene selected from TTYH2 or a gene belonging to the same biosynthetic or regulatory pathway as TTYH2. Also disclosed is the use of the aforementioned polypeptides and polynucleotides in screening for agents that modulate the expression of a gene or the level and or functional activity of an expression product of that gene, wherein the gene is selected from TTYH2 or a gene belonging to the same biosynthetic or regulatory pathway as TTYH2. The invention also discloses the use of such agents for inhibiting or reducing tumorigenesis or for treating and/or preventing conditions that are associated with aberrant TTYH2 expression. Also disclosed are immunopotentiating compositions comprising TTYH2 polynucleotides or TTYH2 polypeptides for eliciting an immune response in a patient, including the production of elements which specifically bind a TTYH2 polypeptide and/or which provide a protective effect against tumorigenesis

### FIELD OF THE INVENTION

THIS INVENTION relates generally to polynucleotides and polypeptides linked to cancer and/or tumorigenesis. More particularly, the present invention relates to novel TTYH2 polynucleotides whose expression is modulated in cancers or tumours and especially in renal cell carcinoma, and to TTYH2 polypeptides encoded thereby. The invention also relates to biologically active fragments of the TTYH2 polypeptides, to variants and derivatives of these polypeptides and to polynucleotides encoding those fragments, variants and derivatives. Further, the invention relates to antigen-binding molecules that are immuno-interactive with the polypeptides of the invention and to the use of these antigen-binding molecules for diagnostic purposes. The invention also encompasses methods for detecting the presence or diagnosing the risk of a cancer or tumour by detecting aberrant expression of a gene selected from TTYH2 or a gene belonging to the same biosynthetic or regulatory pathway as TTYH2. The invention also extends to methods of screening for agents that modulate the expression of a gene or the level and/or functional activity of an expression product of that gene, wherein the gene is selected from TTYH2 or a gene belonging to the same biosynthetic or regulatory pathway as TTYH2. The invention also relates to the use of these modulatory agents in methods for modulating tumorigenesis or for treating and/or preventing a cancer or tumour. Also encompassed are immunopotentiating compositions comprising TTYH2 polynucleotides or TTYH2 polypeptides for eliciting an immune response in a patient, including the production of elements which specifically bind a TTYH2 polypeptide and/or which provide a protective effect against tumorigenesis.

Bibliographic details of various publications referred to in this specification are collected at the end of the description.

### BACKGROUND OF THE INVENTION

A limited number of genetic changes have been identified in renal cell carcinoma (RCC) based on studies of familial forms of this disease. Mutations in the von Hippel Lindau (VHL) gene, a tumour suppressor gene, (Latif et al., 1993; Maher et al., 1991) are associated with familial and many sporadic clear cell RCC, the most common form of RCC. Hereditary papillary RCC has been linked with the c-MET proto-oncogene (Schmidt et al., 1997) and increased expression is also associated with sporadic papillary RCC (Fleming et al., 1998). However, not all patients with RCC have mutations or alterations in the expression of these currently identified genes, as illustrated by other forms of familial RCC (Teh et al., 1997). Thus, it is reasonable to speculate that there are other, potentially functionally significant, genetic and/or molecular abnormalities involved in the initiation and progression of RCC that are yet to be identified.

Tumorigenesis is the result of multiple genetic alterations, which act coordinately to contribute to the disease process. Identification of genes whose expression is dramatically altered in tumour versus normal cells will be invaluable in furthering our understanding of the molecular events underlying cancer development (Sager, 1997). Comparison of cellular gene expression profiles, using techniques such as differential display-polymerase chain reaction (DD-PCR) (Liang & Pardee, 1992), is a valuable tool for isolating disease-associated genes. DD-PCR has been used extensively to identify genes that are differentially expressed in cancers of the breast, prostate and ovary (Chen et al., 1998; Cole et al., 1998; Mok et al., 1998). In comparison, only a small number of studies have used this approach to examine RCC (Ivanov et al., 1998; Kocher et al., 1995; Stassar et al., 1999; Thrash-Bingham & Tartof, 1999). Although a number of genes associated with RCC were identified in these studies, their precise role in RCC tumorigenesis is yet to be elucidated.

In work leading up to the present invention, the inventors sought to identify other genes that are differentially expressed in RCC, by performing DD-PCR using RNA derived from RCC and from normal kidney parenchyma obtained from the same individual. A novel partial gene sequence was identified whose expression was up-regulated in RCC. This gene was cloned and its genomic localisation, structure and tissue expression pattern determined. The predicted 534 amino acid protein shows homology to the human (48%) and mouse (49%) TTYH1 (tweety homologue 1) and Drosophila melanogaster tweety (29%) proteins and thus this novel gene was designated TTYH2 (tweety homologue 2). The mouse orthologue was also identified and shares 81% identity with the human TTYH2 protein. These two novel proteins have 5 transmembrane regions in the same arrangement to the other tweety-related proteins, indicating that they are members of a new family of putative membrane-spanning proteins.

### SUMMARY OF THE INVENTION

Accordingly, in one aspect of the invention, there is provided an isolated polypeptide comprising an amino acid sequence corresponding to at least a biologically active fragment of the sequence set forth in SEQ ID NO: 2 or 7, or to a variant or derivative thereof.

The biologically active fragment preferably comprises at least 6, and more preferably at least 8, contiguous amino acids contained within the sequence set forth in SEQ ID NO: 2 or 7. In one embodiment, the biologically active fragment is selected from residues 1-8, 9-16, 17-24, 25-32, 33-40, 41-48, 49-56, 57-64, 65-72, 73-80, 81-88, 89-96, 97-104, 105-112, 113-120, 121-128, 129-136, 137-144, 145-152, 153-160, 161-168, 169-176, 177-184, 185-192, 193-200, 201-208, 209-216, 217-224, 225-232, 223-240, 241-248, 249-256, 257-264, 265-272,273-280, 281-288, 289-296, 297-304, 305-312, 313-320, 321-328, 329-336, 337-344, 345-352, 353-360, 361-368, 369-376, 317-384, 385-392, 393-400, 401-408, 409-416, 417-424, 425-432, 423-440, 441-448, 449-456, 457-464, 465-472, 473-480,481-488, 489-496, 497-504, 505-512, 513-520, 521-528 and 527-534 of SEQ ID NO: 2. In another embodiment, the biologically active fragment is selected from residues 1-8, 9-16, 17-24, 25-32, 33-40, 41-48, 49-56, 57-64, 65-72, 73-80, 81-88, 89-96, 97-104, 105-112, 113-120, 121-128, 129-136, 137-144, 145-152, 153-160, 161-168, 169-176, 177-184, 185-192, 193-200, 201-208, 209-216, 217-224, 225-232, 223-240, 241-248, 249-256, 257-264, 265-272, 273-280, 281-288, 289-296, 297-304, 305-312, 313-320, 321-328, 329-336, 337-344, 345-352, 353-360, 361-368, 369-376, 377-384, 385-392, 393-400, 401-408, 409-416,417-424, 425-432, 423-440, 441-448, 449-456, 457-464, 465-472, 473-480, 481-488, 489-496, 497-504, 505-512, 513-520, 521-528 and 525-532 of SEQ ID NO: 7.

In another embodiment, the biologically active fragment is selected from residues 1-57, 109-216 or 259-391 of SEQ ID NO: 2 or 7. In this instance, the biologically active fragment suitably comprises a predicted extracellular domain of TTYH2.

In yet another embodiment, the biologically active fragment is selected from residues 58-74, 92-108, 217-233, 240-258 or 392-408 of SEQ ID NO: 2 or 7. In this instance, the biologically active fragment suitably comprises a predicted TTYH2 transmembrane domain.

In yet another embodiment, the biologically active fragment is selected from residues 75-91, 234-239 or 409-534 of SEQ ID NO: 2, or residues 409-532 of SEQ ID NO: 7. In this instance, the biologically active fragment suitably comprises a predicted TTYH2 intracellular domain.

Suitably, the variant has at least 50%, preferably at least 55%, more preferably at least 60%, even more preferably at least 65%, even more preferably at least 70%, even more preferably at least 75%, even more preferably at least 80%, even more preferably at least 85%, even more preferably at least 90% and still even more preferably at least 95% sequence identity to the sequence set forth in any one of SEQ ID NO: 2 and 7 or biologically active fragment thereof. In a preferred embodiment, the variant is distinguished from at least a portion of the sequence set forth in SEQ ID NO: 2 or 7 by the substitution of at least one amino acid residue. In an especially preferred embodiment of this type, the substitution is a conservative substitution.

In another aspect, the invention provides an isolated polynucleotide comprising a nucleotide sequence encoding the polypeptide as broadly described above. In a preferred embodiment, the polynucleotide comprises a nucleotide sequence that corresponds or is complementary to at least a portion of the sequence set forth in SEQ ID NO: 1, 3, 4, 6 or 8, or to a polynucleotide variant thereof.

Preferred portions of the said sequence comprise at least 18, more preferably at least 24, contiguous nucleotides of the sequence set forth in SEQ ID NO: 1, 3, 4, 6 or 8.

In one embodiment, the polynucleotide variant has at least 50%, preferably at least 60%, more preferably at least 70%, more preferably at least 80% and still more preferably at least 90% sequence identity to at least a portion of the sequence set forth in SEQ ID NO: 1, 3, 4, 6 or 8.

The variant may be obtained from any suitable animal. Preferably, the variant is obtained from a mammal.

In another aspect, the invention contemplates a vector comprising a polynucleotide as broadly described above.

In yet another aspect, the invention features an expression vector comprising a polynucleotide as broadly described above wherein the polynucleotide is operably linked to a regulatory polynucleotide.

In a further aspect, the invention provides a host cell containing a vector or expression vector as broadly described above.

The invention also contemplates a method of producing a recombinant polypeptide comprising an amino acid sequence corresponding to at least a biologically active fragment of the sequence set forth in SEQ ID NO: 2 or 7, or to a variant or derivative thereof, said method comprising:
culturing a host cell containing an expression vector as broadly described above such that said recombinant polypeptide is expressed from said polynucleotide; and
isolating said recombinant polypeptide.

In a further aspect, the invention provides a method of producing a biologically active fragment of a polypeptide comprising the sequence set forth in SEQ ID NO: 2 or 7, comprising:
introducing a fragment of the polypeptide or a polynucleotide from which the fragment can be translated into a cell; and
detecting modulation of tumorigenesis, which indicates that said fragment is a biologically active fragment.

In a preferred embodiment, the fragment is present in said cell at a level and/or functional activity that correlates with the presence or risk of a cancer or tumour, which is preferably a cancer or tumour of the kidney and more preferably renal cell carcinoma. For example, that level and/or functional activity may correspond to a level and/or functional activity of a polypeptide comprising the sequence set forth in SEQ ID NO: 2 or 7, which correlates with the presence or risk of said cancer or tumour.

In yet a further aspect, the invention provides a method of producing a polypeptide variant of a parent polypeptide comprising the sequence set forth in SEQ ID NO: 2 or 7, or a biologically active fragment thereof, comprising:
providing a modified polypeptide whose sequence is distinguished from the parent polypeptide by the substitution, deletion or addition of at least one amino acid;
introducing said modified polypeptide or a polynucleotide from which the modified polypeptide can be translated into a cell; and
detecting modulation of tumorigenesis, which indicates that said modified polypeptide is a polypeptide variant.

In yet a further aspect, the invention provides a method of producing a polypeptide variant of a parent polypeptide comprising the sequence set forth in any one of SEQ ID NO: 2 and 7, or a biologically active fragment thereof, comprising:
providing a modified polypeptide whose sequence is distinguished from the parent polypeptide or said biologically active fragment, by the substitution, deletion or addition of at least one amino acid;
contacting the modified polypeptide with an antigen-binding molecule that is immuno-interactive with said parent polypeptide or said biologically active fragment; and
detecting the presence of a complex comprising the antigen-binding molecule and the modified polypeptide, which indicates that said modified polypeptide is a variant.

The present inventors have determined that aberrant expression of TTYH2 is associated with modulation of tumorigenesis. Accordingly, the isolated polypeptides and polynucleotides as broadly described above can be used to provide both drug targets and regulators to promote or inhibit one or more of said activities and to provide diagnostic markers for cancers using, for example, detectable polypeptides and polynucleotides as broadly described above, or using detectable agents which interact specifically with those polypeptides or polynucleotides.

Thus, in another aspect, the invention extends to a method of screening for an agent which modulates tumorigenesis, said method comprising:
contacting a preparation comprising:
   (i) a polypeptide comprising an amino acid sequence corresponding to at least a biologically active fragment of the sequence set forth in SEQ ID NO: 2 or 7, or to a variant or derivative thereof; or
   (ii) a polynucleotide comprising at least a portion of a genetic sequence that regulates said polypeptide, which is operably linked to a reporter gene, with a test agent; and
detecting a change in the level and/or functional activity of said polypeptide, or an expression product of said reporter gene, relative to a normal or reference level and/or functional activity in the absence of said test agent.

In a preferred embodiment, said agent inhibits or otherwise reduces tumorigenesis. In this instance, the method is further characterised by detecting an a reduction in the level and/or functional activity of said polypeptide, or an expression product of said reporter gene, relative to said normal or reference level and/or functional activity.

In another aspect, the invention resides in the use of a polypeptide comprising an amino acid sequence that corresponds to at least a biologically active fragment of the sequence set forth in SEQ ID NO: 2 or 7, or to a variant or derivative thereof, to produce an antigen-binding molecule that is immuno-interactive with said polypeptide.

In yet another aspect, the invention provides antigen-binding molecules that are immuno-interactive with said polypeptide, fragment, variant or derivative.

In another aspect, the invention envisions a method for detecting a specific polypeptide or polynucleotide sequence, comprising detecting a sequence of:
SEQ ID NO: 2, or a fragment thereof at least 6 amino acids in length; or
SEQ ID NO: 7, or a fragment thereof at least 6 amino acids in length; or
SEQ ID NO: 1, or a fragment thereof at least 18 nucleotides in length; or
SEQ ID NO: 3, or a fragment thereof at least 18 nucleotides in length, or
SEQ ID NO: 4, or a fragment thereof at least 18 nucleotides in length; or
SEQ ID NO: 6, or a fragment thereof at least 18 nucleotides in length; or
SEQ ID NO: 8, or a fragment thereof at least 18 nucleotides in length.

In yet another aspect, there is provided a method for detecting a polypeptide as broadly described above, comprising:
detecting expression in a cell of a polynucleotide comprising a nucleotide sequence encoding said polypeptide.

According to another aspect of the invention, there is provided a method of detecting a polypeptide comprising an amino acid sequence corresponding to at least a biologically active fragment of the sequence set forth in SEQ ID NO: 2 or 7, or to a variant or derivative thereof in a biological sample, method comprising:
contacting the sample with an antigen-binding molecule as broadly described above; and
detecting the presence of a complex comprising said antigen-binding molecule and said polypeptide, fragment, variant or derivative in said contacted sample.

In another aspect of the invention, there is provided a method for detecting the presence or diagnosing the risk of a cancer or tumour in a patient, comprising detecting aberrant expression of TTYH2 in a biological sample obtained from said patient.

Aberrant expression of a TTYH2 includes and encompasses (i) an aberrant TTYH2 expression product, which suitably comprises a substitution, deletion and/or addition of one or more subunits (e.g., nucleotides or amino acids) relative to a normal TTYH2 expression product; and (ii) a level and/or functional activity of an expression product of a gene selected from TTYH2 or a gene related to the same biosynthetic or regulatory pathway as TTYH2, which differs from a normal reference level and/or functional activity. In a preferred embodiment, the expression product, which is preferably a TTYH2 expression product is expressed at a higher level and/or functional activity than said normal reference level and/or functional activity.

Thus, in another aspect of the present invention, there is provided a method for detecting the presence or diagnosing the risk of a cancer or tumour in a patient, comprising detecting in a biological sample obtained from said patient an aberrant level and/or functional activity of an expression product of a gene selected from TTYH2 or a gene related to the same regulatory or biosynthetic pathway as TTYH2, which correlates with the presence or risk of said cancer or tumour. In a preferred embodiment, the expression product is expressed at a higher level and/or functional activity than said normal reference level and/or functional activity.

In another aspect, the invention provides a method for diagnosing the progression of a cancer or tumour in a patient, comprising measuring aberrant TTYH2 expression in a biological sample obtained from said patient.

In yet another aspect, the invention contemplates a method for prognostic assessment of a cancer or tumour in a patient, comprising detecting aberrant TTYH2 expression n a biological sample obtained from said patient.

In one embodiment, the method comprises detecting a change in the level and/or functional activity of a target molecule selected from an expression product of a gene selected from TTYH2 or a gene relating to the same regulatory or biosynthetic pathway as TTYH2, wherein the change is relative to a normal reference level and/or functional activity of said expression product.

In a preferred embodiment, the method comprises detecting a change in the level and/or functional activity of an expression product of TTYH2 relative to a corresponding normal reference level and/or functional activity of said expression product.

In yet another aspect, the invention encompasses a method for detecting the presence or diagnosing the risk of a cancer or tumour in a patient, comprising:
providing a biological sample from said patient; and
detecting relative to a normal reference value, an elevation in the level and/or functional activity of a member selected from the group consisting of a polypeptide comprising the sequence set forth in any one of SEQ ID NO: 2 and 7, or variant thereof, and a polynucleotide comprising the sequence set forth in any one of SEQ ID NO: 1, 3, 6 and 8, or variant thereof.

In a further aspect, the invention envisions a method for detecting the presence or diagnosing the risk of a cancer or tumour in a patient, comprising:
providing a biological sample from said patient; and
detecting aberrant expression of a TTYH2 polynucleotide or a TTYH2 polypeptide.

In yet another aspect, the invention encompasses a method for detecting the presence or diagnosing the risk of a cancer or tumour in a patient, comprising:
contacting a biological sample obtained from said patient with an antigen-binding molecule as broadly described above,
measuring the concentration of a complex comprising said antigen-binding molecule and a polypeptide comprising the sequence set forth in SEQ ID NO: 2 or 7, or a variant thereof, in said contacted sample; and
relating said measured complex concentration to the concentration of said polypeptide in said sample, wherein the presence of an elevated concentration relative to a normal reference concentration is indicative of said cancer or tumour.

The cancer or tumour is associated with an organ including, but not restricted to, kidney, brain and testis. In a preferred embodiment, the cancer or tumour is selected from a cancer or tumour of the kidney, more preferably renal cell carcinoma (RCC).

In another aspect, the invention encompasses the use of at least a portion of a TTYH2 expression product as broadly described above, or the use of one or more antigen-binding molecules that are immuno-interactive with a TTYH2 expression product as broadly described above, in the manufacture of a kit for detecting a TTYH2 polynucleotide or a TTYH2 polypeptide or the aberrant expression TTYH2 expression product that correlates with the presence or risk of a cancer or tumour.

In another aspect of the invention, there is provided a method for modulating tumorigenesis, said method comprising introducing into said cell an agent as broadly described above for a time and under conditions sufficient to modulate the level and/or functional activity of TTYH2.

The agent preferably decreases the level and/or functional activity of TTYH2.

In yet another aspect, the invention provides a composition for delaying, repressing or otherwise inhibiting tumorigenesis, comprising an agent that reduces the level and/or functional activity of TTYH2, and optionally a pharmaceutically acceptable carrier.

In another aspect, the invention provides a composition for treatment and/or prophylaxis of a cancer or tumour, comprising an agent that reduces the level and/or functional activity of TTYH2, an optionally a pharmaceutically acceptable carrier.

According to another aspect of the invention, there is provided a method for treatment and/or prophylaxis of a cancer or tumour, said method comprising administering to a patient in need of such treatment an effective amount of an agent that reduces the level and/or functional activity of TTYH2, and optionally a pharmaceutically acceptable carrier.

The invention also encompasses the use of the polypeptide as broadly described above, the polynucleotide as broadly described above, the vectors as broadly described above or the modulatory agents as broadly described above in the study, and modulation of tumorigenesis.

In yet another aspect, the invention contemplates the use of an agent as broadly described above in the manufacture of a medicament for restoring a normal level and/or functional activity of a TTYH2 expression product in a patient, wherein said agent is optionally formulated with a pharmaceutically acceptable carrier.

In even yet another aspect, the invention contemplates the use of the polypeptide as broadly described above, the polynucleotide as broadly described above or the expression vector as broadly described above in the manufacture of a medicament for eliciting an immune response in a patient, including the production of elements which specifically bind said polypeptide and/or which provide a protective effect against tumorigenesis, wherein said agent is optionally formulated with a pharmaceutically acceptable carrier.

In still another aspect, the invention extends to the use of an agent as broadly described above or the use of the polypeptide, fragment, variant or derivative as broadly described above or an expression vector as broadly described above in the manufacture of a medicament for the treatment and/or prophylaxis of a cancer or tumour in a patient, wherein said agent is optionally formulated with a pharmaceutically acceptable carrier.

According to another aspect, the invention contemplates a composition, comprising an immunopotentiating agent selected from the polypeptide as broadly described above, the polynucleotide as broadly described above or the vector or expression vector as broadly described above, together with a pharmaceutically acceptable carrier.

The composition may optionally comprise an adjuvant.

In a further aspect, the invention encompasses a method for modulating an immune response, which response is preferably directed against a cancer or tumour, comprising administering to a patient in need of such treatment an effective amount of an immunopotentiating agent selected from the polypeptide as broadly described above, the polynucleotide as broadly described above or the vector or expression vector as broadly described above.

In still another aspect, the invention encompasses a non-human genetically modified animal model for TTYH2 function, wherein the genetically modified animal is characterised by having an altered TTYH2 gene.

The genetically modified animal may comprise an alteration to its genome, wherein the alteration comprises replacement of an endogenous TTYH2 gene with a foreign TTYH2 gene. Alternatively, the alteration may correspond to a partial or complete loss of function in one or both alleles of the endogenous TTYH2 gene. In a preferred embodiment, the genetically modified animal comprises a disruption in at least one allele of the endogenous TTYH2 gene.

### BRIEF DESCRIPTION OF THE DRAWINGS

FIG. 1 DD-PCR analysis showing the up-regulation of TTYH2 (previously designated DD Band no. 13, (Rae et al., 2000) (arrow) in renal cell carcinoma (R) compared with normal kidney parenchyma (N) in two different patient samples (1 and 2) performed in duplicate. DD-PCR performed on another 2 patients showed the same up-regulation of TTYH2 (data not shown).

FIG. 2A. Nucleotide and deduced amino acid sequence of human TTYH2. Nucleotides are numbered on the left and amino acids on the right. The exon/intron boundaries are marked with arrowheads. The putative ATG start codon at nucleotide 11 is underlined and the poly A+signal at nucleotide 3404 is double underlined. The broken underlined sequence in the 3′UTR is the 235 bp fragment identified by DD-PCR. The potential transmembrane domains are boxed in grey. B. In vitro transcription/translation of TTYH2 cDNA. A 59 kDa protein was generated from a PCR product containing nucleotides 3-1618 of the TTYH2 cDNA. A negative control reaction and a control reaction from luciferase cDNA which gave a protein product were also performed (data not shown). C. Hydrophobicity plot of the deduced TTYH2 protein. Kyte-Doolittle hydropathy analysis was performed using a window of 17 residues (Kyte and Doolittle, 1982). Hydrophobicity is shown on the vertical axis with the hydrophobic side of the plot having a positive value. The horizontal axis represents the amino acid residue number. The 5 peaks correspond to amino acids 58-74, 92-108, 217-233, 240-256 and 392-408 and represent the putative transmembrane domains.

FIG. 3 Alignment (GCG Pileup) of predicted protein sequence of the Drosophila melanogaster tweety gene product (dTTY), the truncated tty2 gene product (dTTY2), human TTYH1 gene product (hTTYH1), mouse TTYH1 gene product (mTTYH1), Macaque TTYH1 gene product (maTTYH1), C. elegans TTYH1 gene product (cTTYH1), human TTYH2 gene product (hTTYH2) and mouse TTYH2 gene product (mTTYH2). Residues are boxed when 5 or more are completely conserved. The 16 absolutely conserved residues are indicated by *. The putative transmembrane regions are shaded.

FIG. 4A. Normal male metaphase chromosomes showing FISH with the TTYH2 probe. FISH signals and the DAPI banding pattern were merged for figure preparation. Hybridisation sites on chromosome 17 are indicated by arrows. B. Exon/intron boundaries of the TTYH2 gene. Exon and intron sequences are shown in upper- and lowercase letters, respectively. The nucleotide consensus sequence of the intron adjoining the splice junctions are shown in boldface type. Sizes of the introns were determined by BLAST analysis of the unordered genomic clone RP11-647F2 (accession no. AC021977) as well as amplification of the remaining introns by PCR of BAC 2514K5 (indicated by *). C. Organisation of the TTYH2 gene. Exons are represented as filled boxes, untranslated regions as unfilled boxes and introns as lines (not to scale).

FIG. 5A. Northern blot analysis of TTYH2 expression in 16 normal human tissues. Hybridisation was performed with a cRNA probe generated from TTYH2 cDNA (nucleotides 980-3420). A b-actin control probe was used to verify equivalent loading of RNA in each lane. B. Graphical representation of signal intensities following hybridisation with a probe generated from nucleotides 980-3420 (EST clone AI623520) of TTYH2 cDNA to a Clontech Multiple Tissue expression array containing poly A+RNA from 76 different human tissues and cell lines. Tissues arrayed: 1, whole brain; 2, cerebral cortex; 3, frontal lobe; 4, parietal lobe; 5, occipital lobe 6, temporal lobe; 7, cerebral cortex; 8, pons; 9, cerebellum, left; 10, cerebellum, right; 11, corpus callosum; 12, amygdala; 13, caudate nucleus; 14, hippocampus; 15, medulla oblongata; 16, putamen; 17, substantia nigra; 18, accumbens nucleus; 19, thalamus; 20, pituitary gland; 21, spinal cord; 22, heart; 23, aorta; 24, atrium, left; 25 atrium, right; 26 ventrical, left; 27, ventrical, right; 28, interventricular septum; 29, apex of heart; 30, oesophagus; 31, stomach; 32, duodenum; 33, jejunum; 34, ileum; 35, ilocecum; 36, appendix; 37, colon, ascending; 38, colon, transverse; 39, colon, descending; 40, rectum; 41, kidney; 42, skeletal muscle; 43, spleen; 44, thymus; 45, peripheral blood leucocyte; 46, lymph node; 47, bone marrow; 48, trachea; 49, lung; 50, placenta; 51, bladder; 52, uterus; 53, prostate; 54, testis; 55, ovary; 56, liver; 57, leukemia, HL-60; 58, pancreas; 59, adrenal gland; 60, thyroid gland; 61, salivary gland; 62, mammary gland; 63, HeLa S3; 64, leukemia, 65, leukemia MOLT-4; 66, Burkitt's lymphoma, 67, Burkitt's lymphoma, Daudi; 68, colorectal adenocarcinoma, Raji; 69, lung carcinoma, 70, fettas brain; 71, foetal heart; 72, foetal kidney; 73, foetal liver; 74, foetal spleen; 75, foetal thymus; 76, foetal lung. C. RT-PCR analysis of 17TTYH2 expression in normal kidney and RCC. RT-PCR was performed on 6 female (1-6) and 6 male (7-12) paired RCC(R) and normal kidney (N) samples as well as 2 renal cell carcinoma cell lines, Caki 1 (C) and SN12K1 (S). The expected PCR product sizes are indicated to the right. B2-microglobulin was used as a control for cDNA synthesis.

### BRIEF DESCRIPTION OF THE SEQUENCES: SUMMARY TABLE

TABLE A

| SEQUENCE ID | DESCRIPTION | LENGTH | |
|---|---|---|---|
| SEQ ID NO: 1 | cDNA sequence of human TTYH2 | 3420 | nts |
| SEQ ID NO: 2 | Polypeptide encoded by SEQ ID NO: 1 | 534 | aa |
| SEQ ID NO: 3 | Human TTYH2 CDS | 1605 | nts |
| SEQ ID NO: 4 | TTYH2 genomic sequence | 47999 | nts |
| SEQ ID NO: 5 | Polypeptide encoded by SEQ ID NO: 4 | 534 | aa |
| SEQ ID NO: 6 | cDNA sequence of murine Ttyh2 | 3408 | nts |
| SEQ ID NO: 7 | Polypeptide encoded by SEQ ID NO: 6 | 532 | aa |
| SEQ ID NO: 8 | Murine Ttyh2 CDS | 1599 | nts |
| SEQ ID NO: 9 | Forward PCR primer comprising the T7 RNA polymerase | 56 | nts |
| | binding site | | |
| SEQ ID NO: 10 | Reverse PCR primer | 25 | nts |
| SEQ ID NO: 11 | First mentioned RT PCR primer on page 84 | 23 | nts |
| SEQ ID NO: 12 | Second mentioned RT PCR primer on page 84 | 22 | nts |
| SEQ ID NO: 13 | Intron A forward primer (Table 1) | 22 | nts |
| SEQ ID NO: 14 | Intron A reverse primer (Table 1) | 21 | nts |
| SEQ ID NO: 15 | Intron B forward primer (Table 1) | 20 | nts |
| SEQ ID NO: 16 | Intron B reverse primer (Table 1) | 20 | nts |
| SEQ ID NO: 17 | Intron C forward primer (Table 1) | 20 | nts |
| SEQ ID NO: 18 | Intron C reverse primer (Table 1) | 20 | nts |
| SEQ ID NO: 19 | Intron D forward primer (Table 1) | 22 | nts |
| SEQ ID NO: 20 | Intron D reverse primer (Table 1) | 20 | nts |
| SEQ ID NO: 21 | Intron F forward primer (Table 1) | 20 | nts |
| SEQ ID NO: 22 | Intron F reverse primer (Table 1) | 20 | nts |
| SEQ ID NO: 23 | Intron K forward primer (Table 1) | 22 | nts |
| SEQ ID NO: 24 | Intron K reverse primer (Table 1) | 22 | nts |
| SEQ ID NO: 25 | Intron L forward primer (Table 1) | 22 | nts |
| SEQ ID NO: 26 | Intron L reverse primer (Table 1) | 19 | nts |
| SEQ ID NO: 27 | Intron M forward primer (Table 1) | 20 | nts |
| SEQ ID NO: 28 | Intron M reverse primer (Table 1) | 21 | nts |
| SEQ ID NO: 29 | Nucleotide sequence at the junction between Intron A and | 20 | nts |
| | Exon 2 of the TTYH2 gene | | |
| SEQ ID NO: 30 | Nucleotide sequence at the junction between Intron B and | 20 | nts |
| | Exon 3 of the TTYH2 gene | | |
| SEQ ID NO: 31 | Nucleotide sequence at the junction between Intron C and | 20 | nts |
| | Exon 4 of the TTYH2 gene | | |
| SEQ ID NO: 32 | Nucleotide sequence at the junction between Intron D and | 20 | nts |
| | Exon 5 of the TTYH2 gene | | |
| SEQ ID NO: 33 | Nucleotide sequence at the junction between Intron E and | 20 | nts |
| | Exon 6 of the TTYH2 gene | | |
| SEQ ID NO: 34 | Nucleotide sequence at the junction between Intron F and | 20 | nts |
| | Exon 7 of the TTYH2 gene | | |
| SEQ ID NO: 35 | Nucleotide sequence at the junction between Intron G and | 20 | nts |
| | Exon 8 of the TTYH2 gene | | |
| SEQ ID NO: 36 | Nucleotide sequence at the junction between Intorn H and | 20 | nts |
| | Exon 9 of the TTYH2 gene | | |
| SEQ ID NO: 37 | Nucleotide sequence at the junction between Intron I and | 20 | nts |
| | Exon 10 of the TTYH2 gene | | |
| SEQ ID NO: 38 | Nucleotide sequence at the junction between Intron J and | 20 | nts |
| | Exon 11 of the TTYH2 gene | | |
| SEQ ID NO: 39 | Nucleotide sequence at the junction between Intron K and | 20 | nts |
| | Exon 12 of the TTYH2 gene | | |
| SEQ ID NO: 40 | Nucleotide sequence at the junction between Intron L and | 20 | nts |
| | Exon 13 of the TTYH2 gene | | |
| SEQ ID NO: 41 | Nucleotide sequence at the junction between Intron M and | 20 | nts |
| | Exon 14 of the TTYH2 gene | | |
| SEQ ID NO: 42 | Nucleotide sequence at the junction between Exon 1 and | 20 | nts |
| | Intron A of the TTYH2 gene | | |
| SEQ ID NO: 43 | Nucleotide sequence at the junction between Exon 2 and | 20 | nts |
| | Intron B of the TTYH2 gene | | |
| SEQ ID NO: 44 | Nucleotide sequence at the junction between Exon 3 and | 20 | nts |
| | Intron C of the TTYH2 gene | | |
| SEQ ID NO: 45 | Nucleotide sequence at the junction between Exon 4 and | 20 | nts |
| | Intron D of the TTYH2 gene | | |
| SEQ ID NO: 46 | Nucleotide sequence at the junction between Exon 5 and | 20 | nts |
| | Intron E of the TTYH2 gene | | |
| SEQ ID NO: 47 | Nucleotide sequence at the junction between Exon 6 and | 20 | nts |
| | Intron F of the TTYH2 gene | | |
| SEQ ID NO: 48 | Nucleotide sequence at the junction between Exon 7 and | 20 | nts |
| | Intron G of the TTYH2 gene | | |
| SEQ ID NO: 49 | Nucleotide sequence at the junction between Exon 8 and | 20 | nts |
| | Intron H of the TTYH2 gene | | |
| SEQ ID NO: 50 | Nucleotide sequence at the junction between Exon 9 and | 20 | nts |
| | Intron I of the TTYH2 gene | | |
| SEQ ID NO: 51 | Nucleotide sequence at the junction between Exon 10 and | 20 | nts |
| | Intron J of the TTYH2 gene | | |
| SEQ ID NO: 52 | Nucleotide sequence at the junction between Exon 11 and | 20 | nts |
| | Intron K of the TTYH2 gene | | |
| SEQ ID NO: 53 | Nucleotide sequence at the junction between Exon 12 and | 20 | nts |
| | Intron L of the TTYH2 gene | | |
| SEQ ID NO: 54 | Nucleotide sequence at the junction between Exon 13 and | 20 | nts |
| | Intron M of the TTYH2 gene | | |

### DETAILED DESCRIPTION OF THE INVENTION

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “agent” is meant a naturally occurring or synthetically produced molecule which interacts either directly or indirectly with a target member, the level and/or functional activity of which is to be modulated.

“Amplification product” refers to a nucleic acid product generated by nucleic acid amplification techniques.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

As used herein, the term “binds specifically” “specifically immuno-interactive” and the like refers to antigen-binding molecules that bind, or are otherwise immuno-interactive with, the polypeptide or polypeptide fragments of the invention but do not significantly bind to, or do not otherwise specifically immuno-interact with, homologous prior art polypeptides.

By “biologically active fragment” is meant a fragment of a full-length parent polypeptide which fragment retains the activity of the parent polypeptide. A biologically active fragment will therefore modulate tumorigenesis, or elicit an immunogenic response to produce elements (e.g., antigen-binding molecules) that specifically bind to the parent polypeptide. As used herein, the term “biologically active fragment” includes deletion mutants and small peptides, for example of at least 8, preferably at least 10, more preferably at least 15, even more preferably at least 20 and even more preferably at least 30 contiguous amino acids, which comprise the above activities. Peptides of this type may be obtained through the application of standard recombinant nucleic acid techniques or synthesised using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a polypeptide of the invention with proteinases such as endoLys-C, endoArg-C, endoGlu-C and staphylococcus V8-protease. The digested fragments can be purified by, for example, high performance liquid chromatographic (HPLC) techniques.

The term “biological sample” as used herein refers to a sample that may be extracted, untreated, treated, diluted or concentrated from an animal. The biological sample may include whole blood, serum, plasma, saliva, urine, sweat, ascitic fluid, peritoneal fluid, synovial fluid, amniotic fluid, cerebrospinal fluid, tissue biopsy, and the like. Suitably, the biological sample is a tissue biopsy, preferably selected from kidney, brain, and testis.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “corresponds to” or “corresponding to” is meant a polynucleotide (a) having a nucleotide sequence that is substantially identical or complementary to all or a portion of a reference polynucleotide sequence or (b) encoding an amino acid sequence identical to an amino acid sequence in a peptide or protein. This phrase also includes within its scope a peptide or polypeptide having an amino acid sequence that is substantially identical to a sequence of amino acids in a reference peptide or protein.

By “derivative” is meant a polypeptide that has been derived from the basic sequence by modification, for example by conjugation or complexing with other chemical moieties or by post-translational modification techniques as would be understood in the art. The term “derivative” also includes within its scope alterations that have been made to a parent sequence including additions, or deletions that provide for functionally equivalent molecules. Accordingly, the term derivative encompasses molecules that will have tumorigenic activity, and the elicitation of an immunogenic response to produce elements (e.g., antigen-binding molecules) that specifically bind to the parent polypeptide.

By “effective amount” in the context of treating or preventing a cancer or tumour, is meant the administration of that amount of modulatory agent that modulates the expression of TTYH2 to an individual in need of such treatment, either in a single dose or as part of a series, that is effective for treatment or prevention of that cancer or tumour. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

“Homology” refers to the percentage number of amino acids that are identical or constitute conservative substitutions as defined in Table B infra. Homology may be determined using sequence comparison programs such as GAP (Deveraux et al. 1984). In this way, sequences of a similar or substantially different length to those cited herein might be compared by insertion of gaps into the alignment, such gaps being determined, for example, by the comparison algorithm used by GAP.

“Hybridisation” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridisation potential of paired nucleotides in complementary nucleic acid strands. Matched nucleotides hybridise efficiently, such as the classical A-T and G-C base pair mentioned above. Mismatches are other combinations of nucleotides that do not hybridise efficiently.

Reference herein to “immuno-interactive” includes reference to any interaction, reaction, or other form of association between molecules and in particular where one of the molecules is, or mimics, a component of the immune system.

By “immuno-interactive fragment” is meant a fragment of the polypeptide set forth in any one of SEQ ID NO: 2 and 7, which fragment elicits an immune response, including the production of elements that specifically bind to said polypeptide, or variant or derivative thereof. As used herein, the term “immuno-interactive fragment” includes deletion mutants and small peptides, for example of at least six, preferably at least 8 and more preferably at least 12, even more preferably at least 15, even more preferably at least 18 and still even more preferably at least 20 contiguous amino acids, which comprise antigenic determinants or epitopes. Several such fragments may be joined together.

By “isolated” is meant material that is substantially or essentially free from components that normally accompany it in its native state. For example, an “isolated polynucleotide”, as used herein, refers to a polynucleotide, which has been purified from the sequences which flank it in a naturally occurring state, e.g., a DNA fragment which has been removed from the sequences which are normally adjacent to the fragment.

By “modulating” is meant increasing or decreasing, either directly or indirectly, the level and/or functional activity of a target molecule. For example, an agent may indirectly modulate the said level/activity by interacting with a molecule other than the target molecule. In this regard, indirect modulation of a gene encoding a target polypeptide includes within its scope modulation of the expression of a first nucleic acid molecule, wherein an expression product of the first nucleic acid molecule modulates the expression of a nucleic acid molecule encoding the target polypeptide.

By “obtained from” is meant that a sample such as, for example, a nucleic acid extract or polypeptide extract is isolated from, or derived from, a particular source of the host. For example, the extract may be obtained from a tissue or a biological fluid isolated directly from the host.

The term “oligonucleotide” as used herein refers to a polymer composed of a multiplicity of nucleotide units (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotides and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule may vary depending on the particular application. An oligonucleotide is typically rather short in length, generally from about 10 to 30 nucleotides, but the term can refer to molecules of any length, although the term “polynucleotide” or “nucleic acid” is typically used for large oligonucleotides.

By “operably linked”, “operably connected”, “operable linkage” and the like is meant a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. “Operably connecting” a promoter to a polynucleotide is meant placing the polynucleotide (e.g., protein encoding polynucleotide or other transcript) under the regulatory control of a promoter, which then controls the transcription and optionally translation of that polynucleotide. In the construction of heterologous promoter/structural gene combinations, it is generally preferred to position a promoter or variant thereof at a distance from the transcription start site of the polynucleotide, which is approximately the same as the distance between that promoter and the gene it controls in its natural setting; i.e.: the gene from which the promoter is derived. As is known in the art, some variation in this distance can be accommodated without loss of function.

The term “patient” refers to patients of human or other mammal and includes any individual it is desired to examine or treat using the methods of the invention. However, it will be understood that “patient” does not imply that symptoms are present. Suitable animals that fall within the scope of the present invention include, but are not restricted to, primates, livestock animals (e.g. sheep, cows, horses, donkeys, pigs), laboratory test animals (e.g. rabbits, mice, rats, guinea pigs, hamsters), companion animals (e.g. cats, dogs) and captive wild animals (e.g. foxes, deer, dingoes, avians and reptiles).

By “pharmaceutically-acceptable carrier” is meant a solid or liquid filler, diluent or encapsulating substance that may be safely used in topical or systemic administration.

The term “polynucleotide” or “nucleic acid” as used herein designates mRNA, RNA, cRNA, cDNA or DNA. The term typically refers to oligonucleotides greater than 30 nucleotides in length. Polynucleotide sequences are understood to encompass complementary strands as well as alternative backbones described herein.

The terms “polynucleotide variant” and “variant” refer to polynucleotides displaying substantial sequence identity with a reference polynucleotide sequence or polynucleotides that hybridise with a reference sequence under stringent conditions that are defined hereinafter. These terms also encompasses polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide. The terms “polynucleotide variant” and “variant” also include naturally occurring allelic variants.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

The term “polypeptide variant” refers to polypeptides in which one or more amino acids have been replaced by different amino acids. It is well understood in the art that some amino acids may be changed to others with broadly similar properties without changing the nature of the activity of the polypeptide (conservative substitutions) as described hereinafter. Accordingly, polypeptide variants as used herein encompass polypeptides that have tumorigenic activity.

By “primer” is meant an oligonucleotide which, when paired with a strand of DNA, is capable of initiating the synthesis of a primer extension product in the presence of a suitable polymerising agent. The primer is preferably single-stranded for maximum efficiency in amplification but may alternatively be double-stranded. A primer must be sufficiently long to prime the synthesis of extension products in the presence of the polymerisation agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotides, although it may contain fewer nucleotides. Primers can be large polynucleotides, such as from about 200 nucleotides to several kilobases or more. Primers may be selected to be “substantially complementary” to the sequence on the template to which it is designed to hybridise and serve as a site for the initiation of synthesis. By “substantially complementary”, it is meant that the primer is sufficiently complementary to hybridise with a target nucleotide sequence. Preferably, the primer contains no mismatches with the template to which it is designed to hybridise but this is not essential. For example, non-complementary nucleotides may be attached to the 5′ end of the primer, with the remainder of the primer sequence being complementary to the template. Alternatively, non-complementary nucleotides or a stretch of non-complementary nucleotides can be interspersed into a primer, provided that the primer sequence has sufficient complementarity with the sequence of the template to hybridise therewith and thereby form a template for synthesis of the extension product of the primer.

“Probe” refers to a molecule that binds to a specific sequence or sub-sequence or other moiety of another molecule. Unless otherwise indicated, the term “probe” typically refers to a polynucleotide probe that binds to another nucleic acid, often called the “target nucleic acid”, through complementary base pairing. Probes may bind target nucleic acids lacking complete sequence complementarity with the probe, depending on the stringency of the hybridisation conditions. Probes can be labelled directly or indirectly.

The term “recombinant polynucleotide” as used herein refers to a polynucleotide formed in vitro by the manipulation of nucleic acid into a form not normally found in nature. For example, the recombinant polynucleotide may be in the form of an expression vector. Generally, such expression vectors include transcriptional and translational regulatory nucleic acid operably linked to the nucleotide sequence.

By “recombinant polypeptide” is meant a polypeptide made using recombinant techniques, i.e., through the expression of a recombinant polynucleotide.

By “reporter molecule” as used in the present specification is meant a molecule that, by its chemical nature, provides an analytically identifiable signal that allows the detection of a complex comprising an antigen-binding molecule and its target antigen. The term “reporter molecule” also extends to use of cell agglutination or inhibition of agglutination such as red blood cells on latex beads, and the like.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997. A detailed discussion of sequence analysis can be found in Unit 19.3 of Ausubel et al., “Current Protocols in Molecular Biology”, John Wiley & Sons Inc, 1994-1998, Chapter 15.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g. A, T, C, G, 1) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software.

“Stringency” as used herein, refers to the temperature and ionic strength conditions, and presence or absence of certain organic solvents, during hybridisation and washing procedures. The higher the stringency, the higher will be the degree of complementarity between immobilised target nucleotide sequences and the labelled probe polynucleotide sequences that remain hybridised to the target after washing.

“Stringent conditions” refers to temperature and ionic conditions under which only nucleotide sequences having a high frequency of complementary bases will hybridise. The stringency required is nucleotide sequence dependent and depends upon the various components present during hybridisation and subsequent washes, and the time allowed for these processes. Generally, in order to maximise the hybridisation rate, non-stringent hybridisation conditions are selected; about 20 to 25° C. lower than the thermal melting point (Tₘ). The Tₘ is the temperature at which 50% of specific target sequence hybridises to a perfectly complementary probe in solution at a defined ionic strength and pH. Generally, in order to require at least about 85% nucleotide complementarity of hybridised sequences, highly stringent washing conditions are selected to be about 5 to 15° C. lower than the Tₘ. In order to require at least about 70% nucleotide complementarity of hybridised sequences, moderately stringent washing conditions are selected to be about 15 to 30° C. lower than the Tₘ. Highly permissive (low stringency) washing conditions may be as low as 50° C. below the Tₘ, allowing a high level of mismatching between hybridised sequences. Those skilled in the art will recognise that other physical and chemical parameters in the hybridisation and wash stages can also be altered to affect the outcome of a detectable hybridisation signal from a specific level of homology between target and probe sequences. Other examples of stringency conditions are described in section 3.2.

By “vector” is meant a nucleic acid molecule, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

As used herein, underscoring or italicising the name of a gene shall indicate the gene, in contrast to its protein product, which is indicated in the absence of any underscoring or italicising. For example, “TTYH2” shall mean TTYH2 gene or cDNA sequence, whereas “TTYH2” shall indicate the protein product of the “TTYH2” gene. The terms “TTYH2” and “TTYH2” also include within their scope mammalian orthologues.

2. Isolated Polypeptides, Biologically Active Fragments, Polypeptide Variants and Derivatives

2.1 Polypeptides of the Invention

The present invention arises in part from the unexpected discovery that aberrant expression of a novel gene, designated TTYH2, is linked to the development and/or progression of a cancer or tumour. The invention, therefore, features an isolated polypeptide, designated TTYH2, comprising the sequence set forth in SEQ ID NO: 2 or 7. SEQ ID NO: 2 corresponds to a putative full-length human polypeptide comprising three putative extracellular domain (from residue 1 to about residue 57, from about residue 109 to about residue 216 and from about residue 259-391), five transmembrane domains (from about residue 58 to about residue 74, from about residue 92 to about residue 108, from about residue 217 to about residue 233, from about residue 240 to about residue 258, and from about residue 392 to about residue 408) and three intracellular domains (from about residue 75 to about residue 91, from about residue 234 to about residue 239 and from about residue 409 through 534). SEQ ID NO: 4 corresponds to a putative full-length mouse polypeptide comprising three putative extracellular domain (from residue 1 to about residue 57, from about residue 109 to about residue 216 and from about residue 259-391), five transmembrane domains (from about residue 58 to about residue 74, from about residue 92 to about residue 108, from about residue 217 to about residue 233, from about residue 240 to about residue 258, and from about residue 392 to about residue 408) and three intracellular domains (from about residue 75 to about residue 91, from about residue 234 to about residue 239 and from about residue 409 through 532).

2.2 Biologically Active Fragments

Biologically active fragments may be produced according to any suitable procedure known in the art. For example, a suitable method may include first producing a fragment of said isolated polypeptide and then testing the fragment for the appropriate biological activity. In one embodiment, biological activity of the fragment may be tested by introducing a fragment of the polypeptide, or a polynucleotide from which the fragment can be expressed, into a cell and detecting tumorigenesis, which indicates that said fragment is a biologically active fragment. Suitable assays for assaying these activities are known to persons of skill in the art. Examples of assays that may be used in accordance with the present invention are described in Section 6. Suitable biologically active fragments may comprises at least 6, preferably at least 8, more preferably at least 20 and even more preferably at least 50 amino acids of the polypeptides described above in Section 2.1.

The invention also extends to biological fragments of the above polypeptides, which can elicit an immune response in an animal and preferably in a heterologous animal from which the polypeptide is obtained. For example exemplary polypeptide fragments of 8 residues in length, which could elicit an immune response, include but are not limited to residues 1-8, 9-16, 17-24, 25-32, 33-40, 41-48, 49-56, 57-64, 65-72, 73-80, 81-88, 89-96, 97-104, 105-112, 113-120, 121-128, 129-136, 137-144, 145-152, 153-160, 161-168, 169-176, 177-184, 185-192, 193-200, 201-208, 209-216, 217-224, 225-232, 223-240, 241-248, 249-256, 257-264, 265-272, 273-280, 281-288, 289-296, 297-304, 305-312, 313-320, 321-328, 329-336, 337-344, 345-352, 353-360, 361-368, 369-376, 377-384, 385-392, 393-400, 401-408, 409-416, 417-424, 425-432, 423-440, 441-448, 449-456, 457-464, 465-472, 473-480, 481-488, 489-496, 497-504, 505-512, 513-520, 521-528 and 527-534 of SEQ ID NO: 2. In an alternate embodiment of this type, the biologically active fragment is selected from residues 1-8, 9-16, 17-24, 25-32, 33-40, 41-48, 49-56, 57-64, 65-72, 73-80, 81-88, 89-96, 97-104, 105-112, 113-120, 121-128, 129-136, 137-144, 145-152, 153-160, 161-168, 169-176, 177-184, 185-192, 193-200, 201-208, 209-216, 217-224, 225-232, 223-240, 241-248, 249-256, 257-264, 265-272, 273-280, 281-288, 289-296, 297-304, 305-312, 313-320, 321-328, 329-336, 337-344, 345-352, 353-360, 361-368, 369-376, 377-384, 385-392, 393-400, 401-408, 409-416, 417-424, 425-432, 423-440, 441-448, 449-456, 457-464, 465-472, 473-480, 481-488, 489-496, 497-504, 505-512, 513-520, 521-528 and 525-532 of SEQ ID NO: 7.

In another embodiment, the biologically active fragment comprises a TTYH2 domain mentioned above. In a preferred embodiment of this type, the biologically active fragment comprises a TTYH2 extracellular domain.

2.3 Polypeptide Variants

The invention also contemplates polypeptide variants of the polypeptides of the invention wherein said variants modulate tumorigenesis. Suitable methods of producing polypeptide variants include replacing at least one amino acid of a parent polypeptide comprising the sequence set forth in any one of SEQ ID NO: 2 or 7, or a biologically active fragment thereof, with a different amino acid to produce a modified polypeptide, and testing said modified polypeptide for tumorigenic activity, which indicates that the modified polypeptide is a polypeptide variant.

In another embodiment, a polypeptide variant is produced by replacing at least one amino acid of a parent polypeptide comprising the sequence set forth in SEQ ID NO: 2 or 7, or a biologically active fragment thereof, with a different amino acid to produce a modified polypeptide, introducing said polypeptide or a polynucleotide from which the fragment can be translated into a cell, and detecting tumorigenesis, which indicates that the modified polypeptide is a polypeptide variant. Examples of assays that may be used in accordance with the present invention are described in Section 6.

In general, variants will be the variant has at least 50%, preferably at least 55%, more preferably at least 60%, even more preferably at least 65%, even more preferably at least 70%, even more preferably at least 75%, even more preferably at least 80%, even more preferably at least 85%, even more preferably at least 90% and still even more preferably at least 95% homologous to a polypeptide as for example shown in SEQ ID NO: 2 or 7, or in fragments thereof. Suitably, the variant has at least 50%, preferably at least 55%, more preferably at least 60%, even more preferably at least 65%, even more preferably at least 70%, even more preferably at least 75%, even more preferably at least 80%, even more preferably at least 85%, even more preferably at least 90% and still even more preferably at least 95% sequence identity to the sequence set forth in SEQ ID NO: 2 or 7.

2.4 Methods of Producing Polypeptide Variants

Polypeptide variants according to the invention can be identified either rationally, or via established methods of mutagenesis (see, for example, Watson, J. D. et al., “MOLECULAR BIOLOGY OF THE GENE”, Fourth Edition, Benjamin/Cummings, Menlo Park, Calif., 1987). Significantly, a random mutagenesis approach requires no a priori information about the gene sequence that is to be mutated. This approach has the advantage that it assesses the desirability of a particular mutant based on its function, and thus does not require an understanding of how or why the resultant mutant protein has adopted a particular conformation. Indeed, the random mutation of target gene sequences has been one approach used to obtain mutant proteins having desired characteristics (Leatherbarrow, R. 1986, J. Prot. Eng. 1: 7-16; Knowles, J. R., 1987, Science 236: 1252-1258; Shaw, W. V., 1987, Biochem. J. 246: 1-17; Gerit, J. A. 1987, Chem. Rev. 87: 1079-1105).

Alternatively, where a particular sequence alteration is desired, methods of site-directed mutagenesis can be employed. Thus, such methods may be used to selectively alter only those amino acids of the protein that are believed to be important (Craik, C. S., 1985, Science 228: 291-297; Cronin, et al., 1988, Biochem. 27: 4572-4579; Wilks, et al., 1988, Science 242: 1541-1544).

Variant peptides or polypeptides, resulting from rational or established methods of mutagenesis or from combinatorial chemistries as are known in the art, may comprise conservative amino acid substitutions. Exemplary conservative substitutions in a polypeptide or polypeptide fragment according to the invention may be made according to the following table:

TABLE B

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile, |
| Phe | Met, Leu, Tyr |
| Ser | Thr |
| Thr | Ser |
| Trp | Tyr |
| Tyr | Trp, Phe |
| Val | Ile, Leu |

Substantial changes in function are made by selecting substitutions that are less conservative than those shown in TABLE B. Other replacements would be non-conservative substitutions and relatively fewer of these may be tolerated. Generally, the substitutions which are likely to produce the greatest changes in a polypeptide's properties are those in which (a) a hydrophilic residue (e.g., Ser or Asn) is substituted for, or by, a hydrophobic residue (e.g., Ala, Leu, Ile, Phe or Val); (b) a cysteine or proline is substituted for, or by, any other residue; (c) a residue having an electropositive side chain (e.g., Arg, His or Lys) is substituted for, or by, an electronegative residue (e.g. Glu or Asp) or (d) a residue having a smaller side chain (e.g., Ala, Ser) or no side chain (e.g. Gly) is substituted for, or by, one having a bulky side chain (e.g., Phe or Trp).

What constitutes suitable variants may be determined by conventional techniques. For example, nucleic acids encoding a polypeptide according to SEQ ID NO: 2 or 7 can be mutated using either random mutagenesis for example using transposon mutagenesis, or site-directed mutagenesis as described, for example, in Section 3.2 infra. Variants can be screened subsequently using the methods, for example, described in Section 6.

2.5 Polypeptide Derivatives

With reference to suitable derivatives of the invention, such derivatives include amino acid deletions and/or additions to a polypeptide, fragment or variant of the invention, wherein said derivatives modulate tumorigenesis. “Additions” of amino acids may include fusion of the polypeptides, fragments and polypeptide variants of the invention with other polypeptides or proteins. For example, it will be appreciated that said polypeptides, fragments or variants may be incorporated into larger polypeptides, and that such larger polypeptides may also be expected to modulate an activity as mentioned above.

The polypeptides, fragments or variants of the invention may be fused to a further protein, for example, which is not derived from the original host. The further protein may assist in the purification of the fusion protein. For instance, a polyhistidine tag or a maltose binding protein may be used in this respect as described in more detail below. Other possible fusion proteins are those which produce an immunomodulatory response. Particular examples of such proteins include Protein A or glutathione S-transferase (GST).

Other derivatives contemplated by the invention include, but are not limited to, modification to side chains, incorporation of unnatural amino acids and/or their derivatives during peptide, polypeptide or protein synthesis and the use of crosslinkers and other methods which impose conformational constraints on the polypeptides, fragments and variants of the invention.

Examples of side chain modifications contemplated by the present invention include modifications of amino groups such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with NaBH₄; reductive alkylation by reaction with an aldehyde followed by reduction with NaBH₄; and trinitrobenzylation of amino groups with 2,4,6-trinitrobenzene sulphonic acid (TNBS).

The carboxyl group may be modified by carbodiimide activation via O-acylisourea formation followed by subsequent derivatisation, by way of example, to a corresponding amide.

The guanidine group of arginine residues may be modified by formation of heterocyclic condensation products with reagents such as 2,3-butanedione, phenylglyoxal and glyoxal.

Sulphydryl groups may be modified by methods such as performic acid oxidation to cysteic acid; formation of mercurial derivatives using 4-chloromercuriphenylsulphonic acid, 4-chloromercuribenzoate; 2-chloromercuri-4-nitrophenol, phenylmercury chloride, and other mercurials; formation of a mixed disulphides with other thiol compounds; reaction with maleimide, maleic anhydride or other substituted maleimide; carboxymethylation with iodoacetic acid or iodoacetamide; and carbamoylation with cyanate at alkaline pH.

Tryptophan residues may be modified, for example, by alkylation of the indole ring with 2-hydroxy-5-nitrobenzyl bromide or sulphonyl halides or by oxidation with N-bromosuccinimide.

Tyrosine residues may be modified by nitration with tetranitromethane to form a 3-nitrotyrosine derivative.

The imidazole ring of a histidine residue may be modified by N-carbethoxylation with diethylpyrocarbonate or by alkylation with iodoacetic acid derivatives.

Examples of incorporating unnatural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids. A list of unnatural amino acids contemplated by the present invention is shown in TABLE C.

TABLE C

| Non-conventional amino acid | Non-conventional amino acid |
|---|---|
| α-aminobutyric acid | L-N-methylalanine |
| α-amino-α-methylbutyrate | L-N-methylarginine |
| aminocyclopropane-carboxylate | L-N-methylasparagine |
| aminoisobutyric acid | L-N-methylaspartic acid |
| aminonorbornyl-carboxylate | L-N-methylcysteine |
| cyclohexylalanine | L-N-methylglutamine |
| cyclopentylalanine | L-N-methylglutamic acid |
| L-N-methylisoleucine | L-N-methylhistidine |
| D-alanine | L-N-methylleucine |
| D-arginine | L-N-methyllysine |
| D-aspartic acid | L-N-methylmethionine |
| D-cysteine | L-N-methylnorleucine |
| D-glutamate | L-N-methylnorvaline |
| D-glutamic acid | L-N-methylornithine |
| D-histidine | L-N-methylphenylalanine |
| D-isoleucine | L-N-methylproline |
| D-leucine | L-N-medlylserine |
| D-lysine | L-N-methylthreonine |
| D-methionine | L-N-methyltryptophan |
| D-ornithine | L-N-methyltyrosine |
| D-phenylalanine | L-N-methylvaline |
| D-proline | L-N-methylethylglycine |
| D-serine | L-N-methyl-t-butylglycine |
| D-threonine | L-norleucine |
| D-tryptophan | L-norvaline |
| D-tyrosine | α-methyl-aminoisobutyrate |
| D-valine | α-methyl-γ-aminobutyrate |
| D-α-methylalanine | α-methylcyclohexylalanine |
| D-α-methylarginine | α-methylcylcopentylalanine |
| D-α-methylasparagine | α-methyl-α-napthylalanine |
| D-α-methylaspartate | α-methylpenicillamine |
| D-α-methylcysteine | N-(4-aminobutyl)glycine |
| D-α-methylglutamine | N-(2-aminoethyl)glycine |
| D-α-methylhistidine | N-(3-aminopropyl)glycine |
| D-α-methylisoleucine | N-amino-α-methylbutyrate |
| D-α-methylleucine | α-napthylalanine |
| D-α-methyllysine | N-benzylglycine |
| D-α-methylmethionine | N-(2-carbamylediyl)glycine |
| D-α-methylornithiine | N-(carbamylmethyl)glycine |
| D-α-methylphenylalanine | N-(2-carboxyethyl)glycine |
| D-α-methylproline | N-(carboxymethyl)glycine |
| D-α-methylserine | N-cyclobutylglycine |
| D-α-methylthreonine | N-cycloheptylglycine |
| D-α-methyltryptophan | N-cyclohexylglycine |
| D-α-methyltyrosine | N-cyclodecylglycine |
| L-α-methylleucine | L-α-methyllysine |
| L-α-methylmethionine | L-α-methylnorleucine |
| L-α-methylnorvatine | L-α-methylornithine |
| L-α-methylphenylalanine | L-α-methylproline |
| L-α-methylserine | L-α-methylthreonine |
| L-α-methyltryptophan | L-α-methyltyrosine |
| L-α-methylvaline | L-N-methylhomophenylalanine |
| N-(N-(2,2-diphenylethyl | N-(N-(3,3-diphenylpropyl |
| carbamylmethyl)glycine | carbamylmethyl)glycine |
| 1-carboxy-1-(2,2-diphenyl-ethyl | |
| amino)cyclopropane | |

Also contemplated is the use of crosslinkers, for example, to stabilise 3D conformations of the polypeptides, fragments or variants of the invention, using homo-bifunctional cross linkers such as bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety or carbodiimide. In addition, peptides can be conformationally constrained, for example, by introduction of double bonds between C_{α} and C_{β} atoms of amino acids, by incorporation of C_{α} and N_{α}-methylamino acids, and by formation of cyclic peptides or analogues by introducing covalent bonds such as forming an amide bond between the N and C termini between two side chains or between a side chain and the N or C terminus of the peptides or analogues. For example, reference may be made to: Marlowe (1993, Biorganic & Medicinal Chemistry Letters 3: 437-44) who describes peptide cyclisation on TFA resin using trimethylsilyl (TMSE) ester as an orthogonal protecting group; Pallin and Tam (1995, J: Chem. Soc. Chem. Comm. 2021-2022) who describe the cyclisation of unprotected peptides in aqueous solution by oxime formation; Algin et al (1994, Tetrahedron Letters 35: 9633-9636) who disclose solid-phase synthesis of head-to-tail cyclic peptides via lysine side-chain anchoring; Kates et al (1993, Tetrahedron Letters 34: 1549-1552) who describe the production of head-to-tail cyclic peptides by three-dimensional solid phase strategy; Tumelty et al (1994, J. Chem. Soc. Chem. Comm. 1067-1068) who describe the synthesis of cyclic peptides from an immobilised activated intermediate, wherein activation of the immobilised peptide is carried out with N-protecting group intact and subsequent removal leading to cyclisation; McMurray et al (1994, Peptide Research 7: 195-206) who disclose head-to-tail cyclisation of peptides attached to insoluble supports by means of the side chains of aspartic and glutamic acid; Hruby et al (1994, Reactive Polymers 22: 231-241) who teach an alternate method for cyclising peptides via solid supports; and Schmidt and Langer (1997, J. Peptide Res. 49: 67-73) who disclose a method for synthesising cyclotetrapeptides and cyclopentapeptides. The foregoing methods may be used to produce conformationally constrained polypeptides that modulate tumorigenesis.

The invention also contemplates polypeptides, fragments or variants of the invention that have been modified using ordinary molecular biological techniques so as to improve their resistance to proteolytic degradation or to optimise solubility properties or to render them more suitable as an immunogenic agent.

2.6 Methods of Preparing the Polypeptides of the Invention

Polypeptides of the inventions may be prepared by any suitable procedure known to those of skill in the art. For example, the polypeptides may be prepared by a procedure including the steps of:
(a) preparing a recombinant polynucleotide comprising a nucleotide sequence encoding a polypeptide comprising the sequence set forth in SEQ ID NO: 2 or 7, or variant or derivative of these, which nucleotide sequence is operably linked to transcriptional and translational regulatory nucleic acid;
(b) introducing the recombinant polynucleotide into a suitable host cell;
(c) culturing the host cell to express recombinant polypeptide from said recombinant polynucleotide; and
(d) isolating the recombinant polypeptide.

Suitably, said nucleotide sequence comprises the sequence set forth in any one of SEQ ID NO: 1, 3, 4, 6 and 8.

The recombinant polynucleotide preferably comprises either an expression vector that may be a self-replicating extra-chromosomal vector such as a plasmid, or a vector that integrates into a host genome.

The transcriptional and translational regulatory nucleic acid will generally be appropriate for the host cell used for expression. Numerous types of appropriate expression vectors and suitable regulatory sequences are known in the art for a variety of host cells. Typically, the transcriptional and translational regulatory nucleic acid may include, but is not limited to, promoter sequences, leader or signal sequences, ribosomal binding sites, transcriptional start and stop sequences, translational start and termination sequences, and enhancer or activator sequences. Constitutive or inducible promoters as known in the art are contemplated by the invention. The promoters may be either naturally occurring promoters, or hybrid promoters that combine elements of more than one promoter.

In a preferred embodiment, the expression vector contains a selectable marker gene to allow the selection of transformed host cells. Selection genes are well known in the art and will vary with the host cell used.

The expression vector may also include a fusion partner (typically provided by the expression vector) so that the recombinant polypeptide of the invention is expressed as a fusion polypeptide with said fusion partner. The main advantage of fusion partners is that they assist identification and/or purification of said fusion polypeptide. In order to express said fusion polypeptide, it is necessary to ligate a polynucleotide according to the invention into the expression vector so that the translational reading frames of the fusion partner and the polynucleotide coincide. Well known examples of fusion partners include, but are not limited to, glutathione-5-transferase (GST), Fc potion of human IgG, maltose binding protein (MBP) and hexahistidine (HIS₆), which are particularly useful for isolation of the fusion polypeptide by affinity chromatography. For the purposes of fusion polypeptide purification by affinity chromatography, relevant matrices for affinity chromatography are glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system. In a preferred embodiment, the recombinant polynucleotide is expressed in the commercial vector pFLAG as described more fully hereinafter. Another fusion partner well known in the art is green fluorescent protein (GFP). This fusion partner serves as a fluorescent “tag” which allows the fusion polypeptide of the invention to be identified by fluorescence microscopy or by flow cytometry. The GFP tag is useful when assessing subcellular localisation of the fusion polypeptide of the invention, or for isolating cells which express the fusion polypeptide of the invention. Flow cytometric methods such as fluorescence activated cell sorting (FACS) are particularly useful in this latter application. Preferably, the fusion partners also have protease cleavage sites, such as for Factor Xₐ or Thrombin, which allow the relevant protease to partially digest the fusion polypeptide of the invention and thereby liberate the recombinant polypeptide of the invention therefrom. The liberated polypeptide can then be isolated from the fusion partner by subsequent chromatographic separation. Fusion partners according to the invention also include within their scope “epitope tags”, which are usually short peptide sequences for which a specific antibody is available. Well known examples of epitope tags for which specific monoclonal antibodies are readily available include c-Myc, influenza virus, haemagglutinin and FLAG tags.

The step of introducing into the host cell the recombinant polynucleotide may be effected by any suitable method including transfection, and transformation, the choice of which will be dependent on the host cell employed. Such methods are well known to those of skill in the art.

Recombinant polypeptides of the invention may be produced by culturing a host cell transformed with an expression vector containing nucleic acid encoding a polypeptide, biologically active fragment, variant or derivative according to the invention. The conditions appropriate for protein expression will vary with the choice of expression vector and the host cell. This is easily ascertained by one skilled in the art through routine experimentation. Suitable host cells for expression may be prokaryotic or eukaryotic. One preferred host cell for expression of a polypeptide according to the invention is a bacterium. The bacterium used may be Escherichia coli. Alternatively, the host cell may be an insect cell such as, for example, SF9 cells that may be utilised with a baculovirus expression system.

The recombinant protein may be conveniently prepared by a person skilled in the art using standard protocols as for example described in Sambrook, et al., MOLECULAR CLONING. A LABORATORY MANUAL (Cold Spring Harbor Press, 1989), in particular Sections 16 and 17; Ausubel et al., CURRENT PROTOCOLS IN MOLECULAR BIOLOGY (John Wiley & Sons, Inc. 1994-1998), in particular Chapters 10 and 16; and Coligan et al., CURRENT PROTOCOLS IN PROTEIN SCIENCE (John Wiley & Sons, Inc. 1995-1997), in particular Chapters 1, 5 and 6.

Alternatively, the polypeptide, fragments, variants or derivatives of the invention may be synthesised using solution synthesis or solid phase synthesis as described, for example, in Chapter 9 of Atherton and Shephard (supra) and in Roberge et al (1995, Science 269: 202).

3. Polynucleotides of the Invention

3.1 Polynucleotides Encoding Polypeptides of the Invention

The invention further provides a polynucleotide that encodes a polypeptide, fragment, variant or derivative as defined above. In one embodiment, the polynucleotide comprises the entire sequence of nucleotides set forth in SEQ ID NO: 1. SEQ ID NO: 1 corresponds to a 3420 bp human TTYH2 cDNA sequence comprising: (1) a 5′ untranslated region from nucleotide 1 through nucleotide 10; (2) an open reading frame from nucleotide 11 through nucleotide 1612; and (3) a 3′ untranslated region from nucleotide 1613 through nucleotide 3420. In an alternate embodiment, the polynucleotide comprises the sequence set forth in SEQ ID NO: 3, which defines the above open reading frame and thus encodes a polypeptide of 534 amino acids.

In an alternate embodiment, the polynucleotide comprises the entire sequence of nucleotides set forth in SEQ ID NO: 4. SEQ ID NO: 4 corresponds to a ˜48,000 bp full-length human TTYH2 genomic sequence. This sequence defines: (1) a first exon from nucleotide <1936 through nucleotide 2074; (2) a first intron from nucleotide 2075 through nucleotide 10376; (3) a second exon from nucleotide 10377 through nucleotide 10549; (4) a second intron from nucleotide 10550 through nucleotide 16622; (5) a third exon from nucleotide 16623 through nucleotide 16734; (6) a third intron from nucleotide 16735 through nucleotide 23223; (7) a fourth exon from nucleotide 23224 through nucleotide 23444; (8) a fourth intron from nucleotide 23445 through nucleotide 28299; (9) a fifth exon from nucleotide 28300 through nucleotide 28395; (10) a fifth intron from nucleotide 28394 through nucleotide 28902; (11) a sixth exon from nucleotide 28903 through nucleotide 28975; (12) a sixth intron from nucleotide 28976 through nucleotide 35372; (13) a seventh exon from nucleotide 35373 through nucleotide 35442; (14) a seventh intron from nucleotide 35443 through nucleotide 35705; (15) an eighth exon from nucleotide 35706 through nucleotide 35761; (16) an eighth intron from nucleotide 35762 through nucleotide 36266; (17) a ninth exon from nucleotide 36267 through nucleotide 36359; (18) a ninth intron from nucleotide 36360 through nucleotide 36591; (19) a tenth exon from nucleotide 36592 through nucleotide 36684; (20) a tenth intron from nucleotide 36685 through nucleotide 38529; (21) an eleventh exon from nucleotide 38530 through nucleotide 38672; (21) an eleventh intron from nucleotide 38673 through nucleotide 39376; (22) a twelfth exon from nucleotide 39377 through nucleotide 39562; (23) a twelfth intron from nucleotide 39563 through nucleotide 40050; (24) a thirteenth exon from nucleotide 40051 through nucleotide 40129; (25) a thirteenth intron from nucleotide 40130 through nucleotide 45672, (26) a fourteenth exon from nucleotide 45673 through nucleotide 47558, (27) a 5′ untranslated region from nucleotide <1936 through nucleotide 1945; (28) a start portion of the open reading frame from nucleotide 1946 through nucleotide 2074; (29) 12 other portions of the open reading frame encoded by exons 2-13, respectively (30) an end portion of the open reading frame from nucleotide 45673 through nucleotide 45750; and (31) a 3′ untranslated region from nucleotide 45751 through nucleotide 47558. The aforementioned open reading frames, when joined together, encode a polypeptide comprising 534 residues as set forth in SEQ ID NO: 2.

The human TTYH2 gene, including its portions and flanking polynucleotide sequences have utility for isolating or otherwise producing polynucleotide sequences, including genomic and cDNA sequences of other animals, which could be taken advantage to produce genetically modified non-human animals. Useful sequences for producing genetically modified animals include, but are not restricted to, open reading frames encoding specific polypeptides or domains, introns, and adjacent 5′ and 3′ non-coding nucleotide sequences involved in the regulation of expression, up to about 1 kb beyond the coding region, but possibly further in either direction. Further, the TTYH2 gene and portions thereof, including exons and introns, have utility in a variety of applications, including its use in identifying aberrant TTYH2 genes and transcripts that may be linked to modulation of tumorigenesis.

In another embodiment, the polynucleotide comprises the entire sequence of nucleotides set forth in SEQ ID NO: 6. SEQ ID NO: 6 corresponds to a 3408 bp mouse TTYH2 cDNA sequence comprising: (1) a 5′ untranslated region from nucleotide 1 through nucleotide 19; (2) an open reading frame from nucleotide 20 through nucleotide 1615; and (3) a 3′ untranslated region from nucleotide 1616 through nucleotide 3408. In an alternate embodiment, the polynucleotide comprises the sequence set forth in SEQ ID NO: 8, which defines said open reading frame and thus encodes a polypeptide of 532 amino acids.

3.2 Polynucleotides Variants

In general, polynucleotide variants according to the invention comprise regions that show at least 50%, preferably at least 55%, more preferably at least 60%, even more preferably at least 65%, even more preferably at least 70%, even more preferably at least 75%, even more preferably at least 80%, even more preferably at least 85%, even more preferably at least 90% and still even more preferably at least 95% sequence identity over a reference polynucleotide sequence of identical size (“comparison window”) or when compared to an aligned sequence in which the alignment is performed by a computer homology program known in the art. What constitutes suitable variants may be determined by conventional techniques. For example, a polynucleotide according to any one of SEQ ID NO: 1, 3, 4, 6 and 8 can be mutated using random mutagenesis (e.g. transposon mutagenesis), oligonucleotide-mediated (or site-directed) mutagenesis, PCR mutagenesis and cassette mutagenesis of an earlier prepared variant or non-variant version of an isolated natural promoter according to the invention.

Oligonucleotide-mediated mutagenesis is a preferred method for preparing nucleotide substitution variants of a polynucleotide of the invention. This technique is well known in the art as, for example, described by Adelman et al. (1983). Briefly, a polynucleotide according to any one of SEQ ID NO: 1, 3, 4, 6 and 8 is altered by hybridising an oligonucleotide encoding the desired mutation to a template DNA, wherein the template is the single-stranded form of a plasmid or bacteriophage containing the unaltered or parent DNA sequence. After hybridisation, a DNA polymerase is used to synthesise an entire second complementary strand of the template that will thus incorporate the oligonucleotide primer, and will code for the selected alteration in said parent DNA sequence.

Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridise properly to the single-stranded DNA template molecule.

The DNA template can be generated by those vectors that are either derived from bacteriophage M13 vectors, or those vectors that contain a single-stranded phage origin of replication as described by Viera et al. (1987). Thus, the DNA that is to be mutated may be inserted into one of the vectors to generate single-stranded template. Production of single-stranded template is described, for example, in Sections 4.21-4.41 of Sambrook et al. (1989, supra).

Alternatively, the single-stranded template may be generated by denaturing double-stranded plasmid (or other DNA) using standard techniques.

For alteration of the native DNA sequence, the oligonucleotide is hybridised to the single-stranded template under suitable hybridisation conditions. A DNA polymerising enzyme, usually the Klenow fragment of DNA polymerase I, is then added to synthesise the complementary strand of the template using the oligonucleotide as a primer for synthesis. A heteroduplex molecule is thus formed such that one strand of DNA encodes the mutated form of the polypeptide or fragment under test, and the other strand (the original template) encodes the native unaltered sequence of the polypeptide or fragment under test. This heteroduplex molecule is then transformed into a suitable host cell, usually a prokaryote such as E. coli. After the cells are grown, they are plated onto agarose plates and screened using the oligonucleotide primer having a detectable label to identify the bacterial colonies having the mutated DNA. The resultant mutated DNA fragments are then cloned into suitable expression hosts such as E. coli using conventional technology and clones that retain the desired antigenic activity are detected. Where the clones have been derived using random mutagenesis techniques, positive clones would have to be sequenced in order to detect the mutation.

Alternatively, linker-scanning mutagenesis of DNA may be used to introduce clusters of point mutations throughout a sequence of interest that has been cloned into a plasmid vector. For example, reference may be made to Ausubel et al., supra, (in particular, Chapter 8.4) which describes a first protocol that uses complementary oligonucleotides and requires a unique restriction site adjacent to the region that is to be mutagenised. A nested series of deletion mutations is first generated in the region. A pair of complementary oligonucleotides is synthesised to fill in the gap in the sequence of interest between the linker at the deletion endpoint and the nearby restriction site. The linker sequence actually provides the desired clusters of point mutations as it is moved or “scanned” across the region by its position at the varied endpoints of the deletion mutation series. An alternate protocol is also described by Ausubel et al., supra, which makes use of site directed mutagenesis procedures to introduce small clusters of point mutations throughout the target region. Briefly, mutations are introduced into a sequence by annealing a synthetic oligonucleotide containing one or more mismatches to the sequence of interest cloned into a single-stranded M13 vector. This template is grown in an E. coli duf⁻ ung⁻ strain, which allows the incorporation of uracil into the template strand. The oligonucleotide is annealed to the template and extended with T4 DNA polymerase to create a double-stranded heteroduplex. Finally, the heteroduplex is introduced into a wild-type E. coli strain, which will prevent replication of the template strand due to the presence of apurinic sites (generated where uracil is incorporated), thereby resulting in plaques containing only mutated DNA.

Region-specific mutagenesis and directed mutagenesis using PCR may also be employed to construct polynucleotide variants according to the invention. In this regard, reference may be made, for example, to Ausubel et al, supra, in particular Chapters 8.2A and 8.5.

Alternatively, suitable polynucleotide sequence variants of the invention may be prepared according to the following procedure:
creating primers which are optionally degenerate wherein each comprises a portion of a reference polynucleotide encoding a reference polypeptide or fragment of the invention, preferably encoding the sequence set forth in SEQ ID NO: 2 or 7;
obtaining a nucleic acid extract from an organism, which is preferably an animal, and more preferably a mammal; and
using said primers to amplify, via nucleic acid amplification techniques, at least one amplification product from said nucleic acid extract, wherein said amplification product corresponds to a polynucleotide variant.

Suitable nucleic acid amplification techniques are well known to the skilled addressee, and include polymerase chain reaction (PCR) as for example described in Ausubel et al. (supra); strand displacement amplification (SDA) as for example described in U.S. Pat. No. 5,422,252; rolling circle replication (RCR) as for example described in Liu et al., (1996) and International application WO 92/01813) and Lizardi et al, (International Application WO 97/19193); nucleic acid sequence-based amplification (NASBA) as for example described by Sooknanan et al, (1994); and Q-β replicase amplification as for example described by Tyagi et al, (1996).

Typically, polynucleotide variants that are substantially complementary to a reference polynucleotide are identified by blotting techniques that include a step whereby nucleic acids are immobilised on a matrix (preferably a synthetic membrane such as nitrocellulose), followed by a hybridisation step, and a detection step. Southern blotting is used to identify a complementary DNA sequence; northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al. (1994-1998, supra) at pages 2.9.1 through 2.9.20.

It will be understood that polynucleotide variants according to the invention will hybridise to a reference polynucleotide under at least low stringency conditions. Reference herein to low stringency conditions include and encompass from at least about 1% v/v to at least about 15% v/v formamide and from at least about 1 M to at least about 2 M salt for hybridisation at 42° C., and at least about 1 M to at least about 2 M salt for washing at 42° C. Low stringency conditions also may include 1% Bovine Serum Albumin (BSA), 1 mM EDTA, 0.5 M NaBPO₄ (pH 7.2), 7% SDS for hybridisation at 65° C., and (i) 2×SSC, 0.1% SDS; or (ii) 0.5% BSA, 1 mM EDTA, 40 mM NaBPO₄ (pH 7.2), 5% SDS for washing at room temperature.

Suitably, the polynucleotide variants hybridise to a reference polynucleotide under at least medium stringency conditions. Medium stringency conditions include and encompass from at least about 16% v/v to at least about 30% v/v formamide and from at least about 0.5 M to at least about 0.9 M salt for hybridisation at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridisation at 65° C., and (i) 2×SSC, 0.1% SDS; or (ii) 0.5% BSA, 1 mM EDTA, 40 mM NaHPO₄ (pH 7.2), 5% SDS for washing at 60-65° C.

Preferably, the polynucleotide variants hybridise to a reference polynucleotide under high stringency conditions. High stringency conditions include and encompass from at least about 31% v/v to at least about 50% v/v formamide and from about 0.01 M to about 0.15 M salt for hybridisation at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridisation at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C.

Other stringent conditions are well known in the art. A skilled addressee will recognise that various factors can be manipulated to optimise the specificity of the hybridisation. Optimisation of the stringency of the final washes can serve to ensure a high degree of hybridisation. For detailed examples, see Ausubel et al., supra at pages 2.10.1 to 2.10.16 and Sambrook et al. (1989, supra) at sections 1.101 to 1.104.

While stringent washes are typically carried out at temperatures from about 42° C. to 68° C., one skilled in the art will appreciate that other temperatures may be suitable for stringent conditions. Maximum hybridisation rate typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel et al., supra at page 2.10.8).

In general, the Tₘ of a perfectly matched duplex of DNA may be predicted as an approximation by the formula:
Tₘ=81.5+16.6(log₁₀M)+0.41(% G+C)−0.63(% formamide)−(600/length)
wherein: M is the concentration of Na⁺, preferably in the range of 0.01 molar to 0.4 molar; % G+C is the sum of guanosine and cytosine bases as a percentage of the total number of bases, within the range between 30% and 75% G+C; % formamide is the percent formamide concentration by volume; length is the number of base pairs in the DNA duplex.

The Tₘ of a duplex DNA decreases by approximately 1° C. with every increase of 1% in the number of randomly mismatched base pairs. Washing is generally carried out at Tₘ−15° C. for high stringency, or Tₘ−30° C. for moderate stringency.

In one example of a hybridisation procedure, a membrane (e.g. a nitrocellulose membrane or a nylon membrane) containing immobilised DNA is hybridised overnight at 42° C. in a hybridisation buffer (50% deionised formamide, 5×SSC, 5× Denhardt's solution (0.1% ficoll, 0.1% polyvinylpyrollidone and 0.1% bovine serum albumin), 0.1% SDS and 200 mg/mL denatured salmon sperm DNA) containing labelled probe. The membrane is then subjected to two sequential medium stringency washes (i.e., 2×SSC, 0.1% SDS for 15 min at 45° C., followed by 2×SSC, 0.1% SDS for 15 min at 50° C.), followed by two sequential higher stringency washes (i.e., 0.2×SSC, 0.1% SDS for 12 min at 55° C. followed by 0.2×SSC and 0.1% SDS solution for 12 min at 65-68° C.

Methods for detecting a labelled polynucleotide hybridised to an immobilised polynucleotide are well known to practitioners in the art. Such methods include autoradiography, phosphorimaging, and chemiluminescent, fluorescent and colorimetric detection.

4. Antigen-Binding Molecules

The invention also contemplates antigen-binding molecules that are immuno-interactive with the aforementioned polypeptides, fragments, variants and derivatives. For example, the antigen-binding molecules may comprise whole polyclonal antibodies. Such antibodies may be prepared, for example, by injecting a polypeptide, fragment, variant or derivative of the invention into a production species, which may include mice or rabbits, to obtain polyclonal antisera. Methods of producing polyclonal antibodies are well known to those skilled in the art. Exemplary protocols which may be used are described for example in Coligan et al., (1991), and Ausubel et al., (1994-1998, supra), in particular Section III of Chapter 11.

In lieu of the polyclonal antisera obtained in the production species, monoclonal antibodies may be produced using the standard method as described, for example, by Köhler and Milstein (1975, Nature 256, 495-497), or by more recent modifications thereof as described, for example, in Coligan et al., (1991, supra) by immortalising spleen or other antibody-producing cells derived from a production species which has been inoculated with one or more of the polypeptides, polypeptide fragments, variants or derivatives of the invention.

The invention also contemplates as antigen-binding molecules Fv, Fab, Fab′ and F(ab′)₂ immunoglobulin fragments or other synthetic antigen-binding molecules such as synthetic stabilised Fv fragments, dAbs, minibodies and the like, which can be produced using routine methods by practitioners in the art.

The antigen-binding molecules of the invention may be used for affinity chromatography in isolating a natural or recombinant polypeptide or biologically active fragment of the invention. For example reference may be made to immunoaffinity chromatographic procedures described in Chapter 9.5 of Coligan et al., (1995-1997, supra). The antigen-binding molecules can be used to screen expression libraries for variant polypeptides of the invention as described herein. They can also be used to detect polypeptides, polypeptide fragments, variants and derivatives of the invention as described hereinafter.

5. Methods of Detecting Aberrant TTYH2 Expression

The present invention is predicated in part on the discovery that patients with a cancer or tumour including, but not limited to, renal cell carcinoma, have aberrant levels of TTYH2 transcripts, and presumably aberrant levels of TTYH2, relative to normal patients. Thus, the invention features a method for detecting the presence or diagnosing the risk of a cancer or tumour in a patient, comprising detecting aberrant expression of a TTYH2 gene in a biological sample obtained from said patient.

In one embodiment, the method comprises detecting a change in the expression of a gene or the level and/or functional activity of an expression product of said gene, wherein the gene is selected from TTYH2 or a gene relating to the same regulatory or biosynthetic pathway as TTYH2, and wherein the change is relative to a normal reference level and/or functional activity. For example, the presence or risk of a cancer or tumour is diagnosed when a TTYH2 gene product is expressed at a detectably higher compared to the level at which it is expressed in normal patients or in patients who are not afflicted with the cancer or tumour. In a preferred embodiment of this type, the method comprises detecting a level and/or functional activity of an expression product of a TTYH2 gene, which is elevated relative to a normal reference level and/or functional activity of said gene. Suitably, the level and/or functional activity of that expression product in the biological sample is at least 110%, more preferably at least 200%, even more preferably at least 300%, even more preferably at least 500%, even more preferably at least 1000%, even more preferably at least 2000%, even more preferably at least 4000%, even more preferably at least 6000%, even more preferably at least 8000%, and still more preferably at least 10,000% of that which is present in a corresponding biological sample obtained from a normal individual or from an individual who is not afflicted with said cancer or tumour. In another embodiment, the method comprises detecting the presence of an aberrant TTYH2 expression product, which correlates with the presence or risk of said cancer or tumour.

Thus, it will be desirable to qualitatively or quantitatively determine TTYH2 protein levels and/or TTYH2 transcription levels. Alternatively or additionally, it may be desirable to search for aberrant TTYH2 structural genes and regulatory regions. Alternatively or additionally, it may be desirable to qualitatively or quantitatively determine the level of an expression product (e.g., transcript, protein) of a gene relating to the same regulatory or biosynthetic pathway as a TTYH2 gene, which can modulate or otherwise influence TTYH2 protein levels and/or TTYH2 transcription levels. Likewise, it may also be desirable to search for an aberrant gene relating to the same regulatory or biosynthetic pathway as a TTYH2 gene.

The biological sample can include any suitable tissue or fluid. Suitably, the biological sample is a tissue biopsy, preferably selected from kidney, brain, and testis.

5.1 Genetic Diagnosis

One embodiment of the instant invention comprises a method for detecting an increase in the expression of a TTYH2 gene by qualitatively or quantitatively determining the transcripts of a TTYH2 gene in a cell (e.g., a kidney cell). Exemplary nucleic acid sequences for TTYH2 mRNA and its corresponding gene are set forth in the enclosed Sequence Listing infra and are summarised in TABLE A supra.

Another embodiment of the instant invention comprises a method for detecting enhancement of expression or function of a TTYH2 gene, by examining a TTYH2 gene and TTYH2 transcripts of a cell. It will also be appreciated that assays may detect or measure modulation of a genetic sequence from which TTYH2 is regulated or expressed. In another example, the subject of detection could be an upstream regulator of TTYH2/TTYH2, or a downstream regulatory target of TTYH2/TTYH2, instead of TTYH2/TTYH2.

Nucleic acid used in polynucleotide-based assays can be isolated from cells contained in the biological sample, according to standard methodologies (Sambrook, et al., “Molecular Cloning. A Laboratory Manual”, Cold Spring Harbor Press, 1989; Ausubel et al., “Current Protocols in Molecular Biology”, John Wiley & Sons Inc, 1994-1998). The nucleic acid may be genomic DNA or fractionated or whole cell RNA. Where RNA is used, it may be desired to convert the RNA to a complementary DNA. In one embodiment, the RNA is whole cell RNA; in another, it is poly-A RNA. In one embodiment, the nucleic acid is amplified by a nucleic acid amplification technique. Suitable nucleic acid amplification techniques are well known to the skilled addressee, and include the polymerase chain reaction (PCR) as for example described in Ausubel et al. (supra); strand displacement amplification (SDA) as for example described in U.S. Pat. No. 5,422,252; rolling circle replication (RCR) as for example described in Liu et al., (1996) and International application WO 92/01813) and Lizardi et al., (International Application WO 97/19193); nucleic acid sequence-based amplification (NASBA) as for example described by Sooknanan et al., (1994, Biotechniques 17: 1077-1080); and Q-β replicase amplification as for example described by Tyagi et al., (1996, Proc. Natl. Acad. Sci. USA 93: 5395-5400).

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994, J Macromol. Sci. Pure, Appl Chem., A31(1): 1355-1376).

Following detection, one may compare the results seen in a given patient with a control reaction or a statistically significant reference group of normal patients. In this way, it is possible to correlate the amount of a TTYH2 detected with the progression or severity of the disease.

In addition to determining levels of TTYH2 transcripts, it also may prove useful to examine various types of defects. These defect could include deletions, insertions, point mutations and duplications. Point mutations result in stop codons, frameshift mutations or amino acid substitutions. Somatic mutations are those occurring in non-germline tissues. Germ-line tissue can occur in any tissue and are inherited. Mutations in and outside the coding region also may affect the amount of TTYH2 produced, both by altering the transcription of the gene or in destabilising or otherwise altering the processing of either the transcript (mRNA) or protein.

A variety of different assays are contemplated in this regard, including but not limited to, fluorescent in situ hybridisation (FISH), direct DNA sequencing, pulse field gel electrophoresis (PFGE) analysis, Southern or Northern blotting, single-stranded conformation analysis (SSCA), RNase protection assay, allele-specific oligonucleotide (ASO), dot blot analysis, denaturing gradient gel electrophoresis, RFLP and PCR-SSCP.

5.1.1 Primers and Probes

Primers may be provided in double-stranded or single-stranded form, although the single-stranded form is preferred. Probes, while perhaps capable of priming, are designed to bind to a target DNA or RNA and need not be used in an amplification process. In preferred embodiments, the probes or primers are labelled with radioactive species ³²P, ¹⁴C, ³⁵S, ³H, or other label), with a fluorophore (rhodamine, fluorescein) or a chemillumiscent label (luciferase).

5.1.2 Template Dependent Amplification Methods

A number of template dependent processes are available to amplify the marker sequences present in a given template sample. An exemplary nucleic acid amplification technique is the polymerase chain reaction (referred to as PCR) which is described in detail in U.S. Pat. Nos. 4,683,195, 4,683,202 and 4,800,159, Ausubel et al. (supra), and in Innis et al., (“PCR Protocols”, Academic Press, Inc., San Diego Calif., 1990).

Briefly, in PCR, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR amplification procedure may be performed in order to quantify the amount of mRNA amplified. Methods of reverse transcribing RNA into cDNA are well known and described in Sambrook et al., 1989. Alternative methods for reverse transcription utilise thermostable, RNA-dependent DNA polymerases. These methods are described in WO 90/07641. Polymerase chain reaction methodologies are well known in the art.

Another method for amplification is the ligase chain reaction (“LCR”), disclosed in EPO No. 320 308. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Qβ Replicase, described in PCT Application No. PCT/US87/00880, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5α-thio-triphosphates in one strand of a restriction site may also be useful in the amplification of nucleic acids in the present invention, Walker et al., (1992, Proc. Natl. Acad. Sci. U.S.A 89: 392-396).

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridised to DNA that is present in a sample. Upon hybridisation, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, may be used in accordance with the present invention. In the former application, “modified” primers are used in a PCR-like, template- and enzyme-dependent synthesis. The primers may be modified by labelling with a capture moiety (e.g., biotin) and/or a detector moiety (e.g., enzyme). In the latter application, an excess of labelled probes are added to a sample. In the presence of the target sequence, the probe binds and is cleaved catalytically. After cleavage, the target sequence is released intact to be bound by excess probe. Cleavage of the labelled probe signals the presence of the target sequence.

Other nucleic acid amplification procedures include transcription-based amplification systems (TAS), including nucleic acid sequence based amplification (NASBA) and 3SR (Kwoh et al., 1989, Proc. Natl. Acad. Sci. U.S.A., 86: 1173; Gingeras et al., PCT Application WO 88/10315). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerisation, DNA/RNA hybrids are digested with RNase H while double stranded DNA molecules are heat denatured again. In either case the single stranded DNA is made fully double stranded by addition of second target specific primer, followed by polymerisation. The double-stranded DNA molecules are then multiply transcribed by an RNA polymerase such as T7 or SP6. In an isothermal cyclic reaction, the RNAs are reverse transcribed into single stranded DNA, which is then converted to double stranded DNA, and then transcribed once again with an RNA polymerase such as T7 or SP6. The resulting products, whether truncated or complete, indicate target specific sequences.

Davey et al., EPO No. 329 822 disclose a nucleic acid amplification process involving cyclically synthesising single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H(RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al. in PCT Application WO 89/06700 disclose a nucleic acid sequence amplification scheme based on the hybridisation of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, M. A., In: “PCR Protocols: A Guide to Methods and Applications”, Academic Press, N.Y., 1990; Ohara et al., 1989, Proc. Natl Acad. Sci. U.S.A., 86: 5673-567).

Methods based on ligation of two (or more) oligonucleotides in the presence of nucleic acid having the sequence of the resulting “di-oligonucleotide”, thereby amplifying the di-oligonucleotide, may also be used in the amplification step of the present invention. Wu et al., (1989, Genomics 4: 560).

5.1.3 Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labelled) under conditions that promote denaturation and rehybridization. Because the probe is designed to base pair with the target, the probe will binding a portion of the target sequence under renaturing conditions. Unbound probe is then removed, and detection is accomplished as described above.

5.1.4 Detection Methods

Products may be visualised in order to confirm amplification of the marker sequences. One typical visualisation method involves staining of a gel with ethidium bromide and visualisation under UV light. Alternatively, if the amplification products are integrally labelled with radio- or fluorometrically-labelled nucleotides, the amplification products can then be exposed to x-ray film or visualised under the appropriate stimulating spectra, following separation.

In one embodiment, visualisation is achieved indirectly. Following separation of amplification products, a labelled nucleic acid probe is brought into contact with the amplified marker sequence. The probe preferably is conjugated to a chromophore but may be radiolabelled. In another embodiment, the probe is conjugated to a binding partner, such as an antibody or biotin, and the other member of the binding pair carries a detectable moiety or reporter molecule.

In one embodiment, detection is by a labelled probe. The techniques involved are well known to those of skill in the art and can be found in many standard texts on molecular protocols. See Sambrook et al., 1989. For example, chromophore or radiolabel probes or primers identify the target during or following amplification.

One example of the foregoing is described in U.S. Pat. No. 5,279,721, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al., 1994, Hum. Mutat. 3: 126-132). The present invention provides methods by which any or all of these types of analyses may be used. Using, for example, the sequences set forth in herein, oligonucleotide primers may be designed to permit the amplification of sequences throughout TTYH2 that may then be analysed by direct sequencing.

5.1.5 Kit Components

All the essential materials and reagents required for detecting and sequencing TTYH2 genes and variants thereof may be assembled together in a kit. The kits may also optionally include appropriate reagents for detection of labels, positive and negative controls, washing solutions, dilution buffers and the like. For example, a nucleic acid-based detection kit may include (i) a polynucleotide according to the invention (which may be used as a positive control), (ii) an oligonucleotide primer according to the invention. Also included may be enzymes suitable for amplifying nucleic acids including various polymerases (Reverse Transcriptase, Taq, Sequenase™ DNA ligase etc. depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe.

5.1.6 Chip Technologies

Also contemplated by the present invention are chip-based DNA technologies such as those described by Hacia et al. (1996, Nature Genetics 14: 441-447) and Shoemaker et al. (1996, Nature Genetics 14: 450-456). Briefly, these techniques involve quantitative methods for analysing large numbers of genes rapidly and accurately. By tagging genes with oligonucleotides or using fixed probe arrays, one can employ chip technology to segregate target molecules as high density arrays and screen these molecules on the basis of hybridisation. See also Pease et al. (1994, Proc. Natl. Acad. Sci. U.S.A. 91: 5022-5026); Fodor et al. (1991, Science 251: 767-773).

5.2 Protein-Based Diagnostics

5.2.1 Antigen-Binding Molecules

Antigen-binding molecules that are immuno-interactive with a target molecule of the present invention can be used in measuring an increase in TTYH2 expression. Thus, the present invention also contemplates antigen-binding molecules that bind specifically to a TTYH2 polypeptide or to proteins that regulate or otherwise influence the level and/or functional activity of a TTYH2 polypeptide.

5.2.2 Immunodiagnostic Assays

The above antigen-binding molecules have utility in measuring directly or indirectly modulation of TTYH2 expression in healthy and diseased states, through techniques such as ELISAs and Western blotting. Illustrative assay strategies which can be used to detect a target polypeptide of the invention include, but are not limited to, immunoassays involving the binding of an antigen-binding molecule to the target polypeptide (e.g., a TTYH2 polypeptide) in the sample, and the detection of a complex comprising the antigen-binding molecule and the target polypeptide. Preferred immunoassays are those that can measure the level and/or functional activity of a target molecule of the invention. Typically, an antigen-binding molecule that is immuno-interactive with a target polypeptide of the invention is contacted with a biological sample suspected of containing said target polypeptide. The concentration of a complex comprising the antigen-binding molecule and the target polypeptide is measure in and the measured complex concentration is then related to the concentration of target polypeptide in the sample. Consistent with the present invention, the presence of an aberrant concentration of the target polypeptide is indicative of the presence of, or probable affliction with, a cancer or tumour.

Any suitable technique for determining formation of an antigen-binding molecule-target antigen complex may be used. For example, an antigen-binding molecule according to the invention, having a reporter molecule associated therewith may be utilised in immunoassays. Such immunoassays include, but are not limited to, radioimmunoassays (RIAs), enzyme-linked immunosorbent assays (ELISAs) and immunochromatographic techniques (ICTs), Western blotting which are well known those of skill in the art. For example, reference may be made to Coligan et al. (1994, supra) which discloses a variety of immunoassays that may be used in accordance with the present invention. Immunoassays may include competitive assays as understood in the art or as for example described infra. It will be understood that the present invention encompasses qualitative and quantitative immunoassays.

Suitable immunoassay techniques are described for example in U.S. Pat. Nos. 4,016,043, 4,424,279 and 4,018,653. These include both single-site and two-site assays of the non-competitive types, as well as the traditional competitive binding assays. These assays also include direct binding of a labelled antigen-binding molecule to a target antigen.

Two site assays are particularly favoured for use in the present invention. A number of variations of these assays exist, all of which are intended to be encompassed by the present invention. Briefly, in a typical forward assay, an unlabelled antigen-binding molecule such as an unlabelled antibody is immobilised on a solid substrate and the sample to be tested brought into contact with the bound molecule. After a suitable period of incubation, for a period of time sufficient to allow formation of an antibody-antigen complex, another antigen-binding molecule, suitably a second antibody specific to the antigen, labelled with a reporter molecule capable of producing a detectable signal is then added and incubated, allowing time sufficient for the formation of another complex of antibody-antigen-labelled antibody. Any unreacted material is washed away and the presence of the antigen is determined by observation of a signal produced by the reporter molecule. The results may be either qualitative, by simple observation of the visible signal, or may be quantitated by comparing with a control sample containing known amounts of antigen. Variations on the forward assay include a simultaneous assay, in which both sample and labelled antibody are added simultaneously to the bound antibody. These techniques are well known to those skilled in the art, including minor variations as will be readily apparent. In accordance with the present invention, the sample is one that might contain an antigen including a tissue or fluid as described above.

In the typical forward assay, a first antibody having specificity for the antigen or antigenic parts thereof is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well known in the art and generally consist of cross-linking covalently binding or physically adsorbing, the polymer-antibody complex is washed in preparation for the test sample. An aliquot of the sample to be tested is then added to the solid phase complex and incubated for a period of time sufficient and under suitable conditions to allow binding of any antigen present to the antibody. Following the incubation period, the antigen-antibody complex is washed and dried and incubated with a second antibody specific for a portion of the antigen. The second antibody has generally a reporter molecule associated therewith that is used to indicate the binding of the second antibody to the antigen. The amount of labelled antibody that binds, as determined by the associated reporter molecule, is proportional to the amount of antigen bound to the immobilised first antibody.

An alternative method involves immobilising the antigen in the biological sample and then exposing the immobilised antigen to specific antibody that may or may not be labelled with a reporter molecule. Depending on the amount of target and the strength of the reporter molecule signal, a bound antigen may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

From the foregoing, it will be appreciated that the reporter molecule associated with the antigen-binding molecule may include the following: (a) direct attachment of the reporter molecule to the antigen-binding molecule; (b) indirect attachment of the reporter molecule to the antigen-binding molecule; i.e., attachment of the reporter molecule to another assay reagent which subsequently binds to the antigen-binding molecule; and (c) attachment to a subsequent reaction product of the antigen-binding molecule.

The reporter molecule may be selected from a group including a chromogen, a catalyst, an enzyme, a fluorochrome, a chemiluminescent molecule, a lanthanide ion such as Europium (Eu³⁴), a radioisotope and a direct visual label.

In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes suitable for use as reporter molecules is disclosed in United States Patent Specifications U.S. Pat. No. 4,366,241, U.S. Pat. No. 4,843,000, and U.S. Pat. No. 4,849,338. Suitable enzymes useful in the present invention include alkaline phosphatase, horseradish peroxidase, luciferase, β-galactosidase, glucose oxidase, lysozyme, malate dehydrogenase and the like. The enzymes may be used alone or in combination with a second enzyme that is in solution.

Suitable fluorochromes include, but are not limited to, fluorescein isothiocyanate (FITC), tetramethylrhodamine isothiocyanate (TRITC), R-Phycoerythrin (RPE), and Texas Red. Other exemplary fluorochromes include those discussed by Dower et al. (International Publication WO 93/06121). Reference also may be made to the fluorochromes described in U.S. Pat. No. 5,573,909 (Singer et al), U.S. Pat. No. 5,326,692 (Brinkley et al). Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218.

In the case of an enzyme immunoassay, an enzyme is conjugated to the second antibody, generally by means of glutaraldehyde or periodates. As will be readily recognised, however, a wide variety of different conjugation techniques exist which are readily available to the skilled artisan. The substrates to be used with the specific enzymes are generally chosen for the production of, upon hydrolysis by the corresponding enzyme, a detectable colour change. Examples of suitable enzymes include those described supra. It is also possible to employ fluorogenic substrates, which yield a fluorescent product rather than the chromogenic substrates noted above. In all cases, the enzyme-labelled antibody is added to the first antibody-antigen complex. It is then allowed to bind, and excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample.

Alternately, fluorescent compounds, such as fluorescein, rhodamine and the lanthanide, europium (EU), may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. The fluorescent-labelled antibody is allowed to bind to the first antibody-antigen complex. After washing off the unbound reagent, the remaining tertiary complex is then exposed to light of an appropriate wavelength. The fluorescence observed indicates the presence of the antigen of interest. Immunofluorometric assays (IFMA) are well established in the art. However, other reporter molecules, such as radioisotope, chemiluminescent or bioluminescent molecules may also be employed.

It will be well understood that other means of testing target polypeptide (e.g., TTYH2) levels are available, including, for instance, those involving testing for an altered level of TTYH2 binding activity to an integrin, or Western blot analysis of TTYH2 protein levels in tissues, cells or fluids using anti-TTYH2 antigen-binding molecule, or assaying the amount of antigen-binding molecule or other TTYH2 binding partner which is not bound to a sample, and subtracting from the total amount of antigen-binding molecule or binding partner added.

6. Identification of Target Molecule Modulators

The invention also features a method of screening for an agent that modulates the expression of a gene or the level and/or functional activity of an expression product that gene, wherein the gene is selected from TTYH2 or a gene relating to the same regulatory or biosynthetic pathway as TTYH2. The method comprises contacting a preparation comprising (i) at least a portion of said expression product or variant or derivative thereof, or (ii) at least a portion of a genetic sequence, which regulates the expression of said gene, in operable linkage with a reporter polynucleotide, with a test agent, and detecting a change in the level and/or functional activity of an expression product produced from (i) or (ii) relative to a normal or reference level and/or functional activity in the absence of said test agent.

In accordance with the present invention, aberrant expression of TTYH2 correlates with the presence or risk of tumorigenesis. Thus, any suitable assay for detecting, measuring or otherwise determining modulation of tumorigenesis is contemplated by the present invention. Assays of a suitable nature are known to persons of skill in the art. It will be understood, in this regard, that the present invention is not limited to the use or practice of any one particular assay for determining a said activity.

Tumorigenesis is typically associated with promotion of cell proliferation. Generally, for cell proliferation, cell number is determined, directly, by microscopic or electronic enumeration, or indirectly, by the use of chromogenic dyes, incorporation of radioactive precursors or measurement of metabolic activity of cellular enzymes. An exemplary cell proliferation assay comprises culturing cells in the presence or absence of a test compound, and detecting cell proliferation by, for example, measuring incorporation of tritiated thymidine or by colorimetric assay based on the metabolic breakdown of 3-(4,5-dimethylthiazol-2-yl)-2,5-diphenyl tetrazolium bromide (MTT) (Mosman, 1983, J. Immunol. Meth. 65: 55-63).

Compounds of interest may be tested for suitability as inhibitors of cell proliferation and enhancers of differentiation using cultured human keratinocytes, as described, for example, in U.S. Pat. No. 5,037,816. Those compounds which inhibit proliferation and induce differentiation in cultured keratinocytes are those potentially useful as therapeutic agents in treating disorders, e.g., precancer, such as actinic keratoses, and cancer, where suppression of cell proliferation is desired.

Cancer or tumour markers are known for a variety of cell or tissue types. Cells or tissues expressing cancer or tumour markers may be detected using monoclonal antibodies, polyclonal antisera or other antigen-binding molecules that are immuno-interactive with these markers or by using nucleic acid analysis techniques, including, for example, detecting the level or presence of marker-encoding polynucleotides.

Modulatory compounds contemplated by the present invention includes agonists and antagonists of TTYH2 gene expression. Antagonists of TTYH2 gene expression include antisense molecules, ribozymes and co-suppression molecules. Agonists include molecules which increase promoter activity or interfere with negative mechanisms. Agonists of TTYH2 include molecules which overcome any negative regulatory mechanism. Antagonists of TTYH2 polypeptides include antibodies and inhibitor peptide fragments.

Candidate agents encompass numerous chemical classes, though typically they are organic molecules, preferably small organic compounds having a molecular weight of more than 50 and less than about 2,500 Dalton. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogues or combinations thereof.

Small (non-peptide) molecule modulators of TTYH2 are particularly preferred. In this regard, small molecules are particularly preferred because such molecules are more readily absorbed after oral administration, have fewer potential antigenic determinants, and/or are more likely to cross the cell membrane than larger, protein-based pharmaceuticals. Small organic molecules may also have the ability to gain entry into an appropriate cell and affect the expression of a gene (e.g. by interacting with the regulatory region or transcription factors involved in gene expression); or affect the activity of a gene by inhibiting or enhancing the binding of accessory molecules.

Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogues. Screening may also be directed to known pharmacologically active compounds and chemical analogues thereof.

Screening for modulatory agents according to the invention can be achieved by any suitable method. For example, the method may include contacting a cell comprising a polynucleotide corresponding to a TTYH2 gene or to a gene belonging to the same regulatory or biosynthetic pathway as TTYH2, with an agent suspected of having said modulatory activity and screening for the modulation of the level and/or functional activity of a protein encoded by said polynucleotide, or the modulation of the level of an expression product encoded by the polynucleotide, or the modulation of the activity or expression of a downstream cellular target of said protein or said expression product Detecting such modulation can be achieved utilising techniques including, but not restricted to, ELISA, cell-based ELISA, filter-binding ELISA, inhibition ELISA, Western blots, immunoprecipitation, slot or dot blot assays, immunostaining, RIA, scintillation proximity assays, fluorescent immunoassays using antigen-binding molecule conjugates or antigen conjugates of fluorescent substances such as fluorescein or rhodamine, Ouchterlony double diffusion analysis, immunoassays employing an avidin-biotin or a streptavidin-biotin detection system, and nucleic acid detection assays including reverse transcriptase polymerase chain reaction (RT-PCR).

It will be understood that a polynucleotide from which a target molecule of interest is regulated or expressed may be naturally occurring in the cell which is the subject of testing or it may have been introduced into the host cell for the purpose of testing. Further, the naturally-occurring or introduced sequence may be constitutively expressed—thereby providing a model useful in screening for agents which down-regulate expression of an encoded product of the sequence wherein said down regulation can be at the nucleic acid or expression product level—or may require activation—thereby providing a model useful in screening for agents that up-regulate expression of an encoded product of the sequence. Further, to the extent that a polynucleotide is introduced into a cell, that polynucleotide may comprise the entire coding sequence which codes for a target protein or it may comprise a portion of that coding sequence (e.g. a domain such as a protein binding domain) or a portion that regulates expression of a product encoded by the polynucleotide (e.g., a promoter). For example, the promoter that is naturally associated with the polynucleotide may be introduced into the cell that is the subject of testing. In this regard, where only the promoter is utilised, detecting modulation of the promoter activity can be achieved, for example, by operably linking the promoter to a suitable reporter polynucleotide including, but not restricted to, green fluorescent protein (GFP), luciferase, β-galactosidase and catecholamine acetyl transferase (CAT). Modulation of expression may be determined by measuring the activity associated with the reporter polynucleotide.

In another example, the subject of detection could be a downstream regulatory target of the target molecule, rather than target molecule itself or the reporter molecule operably linked to a promoter of a gene encoding a product the expression of which is regulated by the target protein.

These methods provide a mechanism for performing high throughput screening of putative modulatory agents such as proteinaceous or non-proteinaceous agents comprising synthetic, combinatorial, chemical and natural libraries. These methods will also facilitate the detection of agents which bind either the polynucleotide encoding the target molecule or which modulate the expression of an upstream molecule, which subsequently modulates the expression of the polynucleotide encoding the target molecule. Accordingly, these methods provide a mechanism of detecting agents that either directly or indirectly modulate the expression and/or activity of a target molecule according to the invention.

In a series of preferred embodiments, the present invention provides assays for identifying small molecules or other compounds (i.e., modulatory agents) which are capable of inducing or inhibiting the level and/or or functional activity of target molecules according to the invention. The assays may be performed in vitro using non-transformed cells, immortalised cell lines, or recombinant cell lines. In addition, the assays may detect the presence of increased or decreased expression of genes or production of proteins on the basis of increased or decreased mRNA expression (using, for example, the nucleic acid probes disclosed herein), increased or decreased levels of protein products (using, for example, the antigen binding molecules disclosed herein), or increased or decreased levels of expression of a reporter gene (e.g., GFP, β-galactosidase or luciferase) operatively linked to a target molecule-related gene regulatory region in a recombinant construct.

Thus, for example, one may culture cells which produce a particular target molecule and add to the culture medium one or more test compounds. After allowing a sufficient period of time (e.g., 6-72 hours) for the compound to induce or inhibit the level and/or functional activity of the target molecule, any change in said level from an established baseline may be detected using any of the techniques described above and well known in the art In particularly preferred embodiments, the cells are selected from kidney cells, brain cells or testicular cells. Using the nucleic acid probes and/or antigen-binding molecules disclosed herein, detection of changes in the level and or functional activity of a target molecule, and thus identification of the compound as agonist or antagonist of the target molecule, requires only routine experimentation.

In particularly preferred embodiments, a recombinant assay is employed in which a reporter gene encoding, for example, GFP, β-galactosidase or luciferase is operably linked to the 5′ regulatory regions of a target molecule related gene. Such regulatory regions may be easily isolated and cloned by one of ordinary skill in the art in light of the present disclosure. The reporter gene and regulatory regions are joined in-frame (or in each of the three possible reading frames) so that transcription and translation of the reporter gene may proceed under the control of the regulatory elements of the target molecule related gene. The recombinant construct may then be introduced into any appropriate cell type although mammalian cells are preferred, and human cells are most preferred. The transformed cells may be grown in culture and, after establishing the baseline level of expression of the reporter gene, test compounds may be added to the medium. The ease of detection of the expression of the reporter gene provides for a rapid, high throughput assay for the identification of agonists or antagonists of the target molecules of the invention.

Compounds identified by this method will have potential utility in modifying the expression of target molecule related genes in vivo. These compounds may be further tested in the animal models to identify those compounds having the most potent in vivo effects. In addition, as described above with respect to small molecules having target polypeptide binding activity, these molecules may serve as “lead compounds” for the further development of pharmaceuticals by, for example, subjecting the compounds to sequential modifications, molecular modelling, and other routine procedures employed in rational drug design.

In another embodiment, a method of identifying agents that inhibit TTYH2 activity is provided in which a purified preparation of TTYH2 protein is incubated in the presence and absence of a candidate agent under conditions in which TTYH2 is active, and the level of TTYH2 activity is measured by a suitable assay. For example, a TTYH2 inhibitor can be identified by measuring the ability of a candidate agent to decrease TTYH2 activity in a cell (e.g., a kidney cell, a brain cell or a testicular cell). In this method, a cell that is capable of expressing TTYH2 is exposed to, or cultured in the presence and absence of, the candidate agent under conditions in which TTYH2 is active in the cell, and tumorigenesis is detected. An agent tests positive if it inhibits any of these activities.

In yet another embodiment, random peptide libraries consisting of all possible combinations of amino acids attached to a solid phase support may be used to identify peptides that are able to bind to a target molecule or to a functional domain thereof. Identification of molecules that are able to bind to a target molecule may be accomplished by screening a peptide library with a recombinant soluble target molecule. The target molecule may be purified, recombinantly expressed or synthesised by any suitable technique. Such molecules may be conveniently prepared by a person skilled in the art using standard protocols as for example described in Sambrook, et al., MOLECULAR CLONING. A LABORATORY MANUAL (Cold Spring Harbor Press, 1989), in particular Sections 16 and 17; Ausubel et al., CURRENT PROTOCOLS IN MOLECULAR BIOLOGY (John Wiley & Sons, Inc. 1994-1998), in particular Chapters 10 and 16; and Coligan et al., CURRENT PROTOCOLS IN PROTEIN SCIENCE (John Wiley & Sons, Inc. 1995-1997), in particular Chapters 1, 5 and 6. Alternatively, a target polypeptide according to the invention may be synthesised using solution synthesis or solid phase synthesis as described, for example, in Chapter 9 of Atherton and Shephard (supra) and in Roberge et al (1995, Science 269: 202).

To identify and isolate the peptide/solid phase support that interacts and forms a complex with a target molecule, preferably a target polypeptide, it may be necessary to label or “tag” the target polypeptide. The target polypeptide may be conjugated to any suitable reporter molecule, including enzymes such as alkaline phosphatase and horseradish peroxidase and fluorescent reporter molecules such as fluorescein isothyiocynate (FITC), phycoerythrin (PE) and rhodamine. Conjugation of any given reporter molecule, with target polypeptide, may be performed using techniques that are routine in the art. Alternatively, target polypeptide expression vectors may be engineered to express a chimeric target polypeptide containing an epitope for which a commercially available antigen-binding molecule exists. The epitope specific antigen-binding molecule may be tagged using methods well known in the art including labelling with enzymes, fluorescent dyes or coloured or magnetic beads.

For example, the “tagged” target polypeptide conjugate is incubated with the random peptide library for 30 minutes to one hour at 22° C. to allow complex formation between target polypeptide and peptide species within the library. The library is then washed to remove any unbound target polypeptide. If the target polypeptide has been conjugated to alkaline phosphatase or horseradish peroxidase the whole library is poured into a petri dish containing a substrate for either alkaline phosphatase or peroxidase, for example, 5-bromo-4-chloro-3-indoyl phosphate (BCIP) or 3,3′,4,4″-diamnobenzidine (DAB), respectively. After incubating for several minutes, the peptide/solid phase-target polypeptide complex changes colour, and can be easily identified and isolated physically under a dissecting microscope with a micromanipulator. If a fluorescently tagged target polypeptide has been used, complexes may be isolated by fluorescent activated sorting. If a chimeric target polypeptide having a heterologous epitope has been used, detection of the peptide/target polypeptide complex may be accomplished by using a labelled epitope specific antigen-binding molecule. Once isolated, the identity of the peptide attached to the solid phase support may be determined by peptide sequencing.

7. Method of Modulating a TTYH2-Related Activity

The invention, therefore, provides a method for modulating tumorigenesis, comprising contacting a cell with an agent for a time and under conditions sufficient to modulate the level and/or functional activity of a polypeptide as broadly described above. In a preferred embodiment, the agent decreases the level and/or functional activity of TTYH2 protein. In such a case, the agent is suitably used to reduce, repress or otherwise inhibit tumorigenesis. Suitable TTYH2 inhibitors may be identified or produced by methods for example disclosed in Section 6.

For example, a suitable TTYH2 inhibitor may comprise oligoribonucleotide sequences, that include anti-sense RNA and DNA molecules and ribozymes that function to inhibit the translation of TTYH2 protein-encoding mRNA. Anti-sense RNA and DNA molecules act to directly block the translation of mRNA by binding to targeted mRNA and preventing protein translation. In regard to antisense DNA, oligodeoxyribonucleotides derived from the translation initiation site, e.g., between −10 and +10 regions of a gene encoding a polypeptide according to the invention, are preferred. Ribozymes are enzymatic RNA molecules capable of catalysing the specific cleavage of RNA. The mechanism of ribozyme action involves sequence specific hybridisation of the ribozyme molecule to complementary target RNA, followed by a endonucleolytic cleavage. Within the scope of the invention are engineered hammerhead motif ribozyme molecules that specifically and efficiently catalyse endonucleolytic cleavage of TTYH2 RNA sequences.

Specific ribozyme cleavage sites within any potential RNA target are initially identified by scanning the target molecule for ribozyme cleavage sites, which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridisation with complementary oligonucleotides, using ribonuclease protection assays.

Both anti-sense RNA and DNA molecules and ribozymes may be prepared by any method known in the art for the synthesis of RNA molecules. These include techniques for chemically synthesising oligodeoxyribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesise antisense RNA constitutively or inducibly, depending on the promoter used, can be introduced stably into cell lines.

Various modifications to the DNA molecules may be introduced as a means of increasing intracellular stability and half-life. Possible modifications include but are not limited to the addition of flanking sequences of ribo- or deoxy-nucleotides to the 5′ and/or 3′ ends of the molecule or the use of phosphorothioate or 2′ O-methyl rather than phosphodiesterase linkages within the oligodeoxyribonucleotide backbone.

8. TTYH2-Modulating Compositions and Uses Therefor

A further feature of the invention is the use of a modulatory agent identified according to Section 6 as actives (“therapeutic agents”) in pharmaceutical compositions for treatment or prophylaxis of a cancer or tumour. The invention, therefore, also extends to a method for treating or preventing a cancer or tumour, comprising administering to a patient in need of such treatment an effective amount of a modulatory agent as broadly described above. The cancer includes, but is not limited to, a cancer of the brain, testis or kidney. In a preferred embodiment, the cancer is a caner of the kidney, more preferably renal cell carcinoma.

A pharmaceutical composition according to the invention is administered to a patient, preferably prior to such symptomatic state associated with the cancer or tumour. The therapeutic agent present in the composition is provided for a time and in a quantity sufficient to treat that patient. Suitably, the pharmaceutical composition comprises a pharmaceutically acceptable carrier.

Depending on the specific conditions being treated, therapeutic agents may be formulated and administered systemically or locally. Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the therapeutic agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art. Intra-muscular and subcutaneous injection is appropriate, for example, for administration of immunogenic compositions, vaccines and DNA vaccines.

The agents can be formulated readily using pharmaceutically acceptable carriers well known in the art into dosages suitable for oral administration. Such carriers enable the compounds of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Pharmaceutical compositions suitable for use in the present invention include compositions wherein the active ingredients are contained in an effective amount to achieve its intended purpose. The dose of agent administered to a patient should be sufficient to effect a beneficial response in the patient over time such as a reduction in the symptoms associated with the cancer or tumour. The quantity of the agent(s) to be administered may depend on the subject to be treated inclusive of the age, sex, weight and general health condition thereof. In this regard, precise amounts of the agent(s) for administration will depend on the judgement of the practitioner. In determining the effective amount of the agent to be administered in the treatment or prophylaxis of the condition, the physician may evaluate tissue levels of a polypeptide, fragment, variant or derivative of the invention, and progression of the disorder. In any event, those of skill in the art may readily determine suitable dosages of the therapeutic agents of the invention.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilisers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more therapeutic agents as described above with the carrier which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilising processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterise different combinations of active compound doses.

Pharmaceutical which can be used orally include push-fit capsules made of gelatin, as well as soft, sealed capsules made of gelatin and a plasticiser, such as glycerol or sorbitol. The push-fit capsules can contain the active ingredients in admixture with filler such as lactose, binders such as starches, and/or lubricants such as talc or magnesium stearate and, optionally, stabilisers. In soft capsules, the active compounds may be dissolved or suspended in suitable liquids, such as fatty oils, liquid paraffin, or liquid polyethylene glycols. In addition, stabilisers may be added.

Dosage forms of the therapeutic agents of the invention may also include injecting or implanting controlled releasing devices designed specifically for this purpose or other forms of implants modified to act additionally in this fashion. Controlled release of an agent of the invention may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be effected by using other polymer matrices, liposomes and/or microspheres.

Therapeutic agents of the invention may be provided as salts with pharmaceutically compatible counterions. Pharmaceutically compatible salts may be formed with many acids, including but not limited to hydrochloric, sulphuric, acetic, lactic, tartaric, malic, succinic, etc. Salts tend to be more soluble in aqueous or other protonic solvents that are the corresponding free base forms.

For any compound used in the method of the invention, the therapeutically effective dose can be estimated initially from cell culture assays. For example, a dose can be formulated in animal models to achieve a circulating concentration range that includes the IC50 as determined in cell culture (e.g., the concentration of a test agent, which achieves a half-maximal inhibition or enhancement of TTYH2 activity). Such information can be used to more accurately determine useful doses in humans.

Toxicity and therapeutic efficacy of such therapeutic agents can be determined by standard pharmaceutical procedures in cell cultures or experimental animals, e.g. for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilised. The exact formulation, route of administration and dosage can be chosen by the individual physician in view of the patient's condition. (See for example Fingl et al., 1975, in “The Pharmacological Basis of Therapeutics”, Ch. 1 p1).

Dosage amount and interval may be adjusted individually to provide plasma levels of the active agent which are sufficient to maintain TTYH2-inhibitory effects. Usual patient dosages for systemic administration range from 1-2000 mg/day, commonly from 1-250 mg/day, and typically from 10-150 mg/day. Stated in terms of patient body weight, usual dosages range from 0.02-25 mg/kg/day, commonly from 0.02-3 mg/kg/day, typically from 0.2-1.5 mg/kg/day. Stated in terms of patient body surface areas, usual dosages range from 0.5-1200 mg/m²/day, commonly from 0.5-150 mg/m²/day, typically from 5-100 mg/m²/day.

Alternately, one may administer the compound in a local rather than systemic manner, for example, via injection of the compound directly into a tissue, which is preferably a kidney tissue, a stomach tissue or a rectal tissue, often in a depot or sustained release formulation. Furthermore, one may administer the drug in a targeted drug delivery system, for example, in a liposome coated with tissue-specific antibody. The liposomes will be targeted to and taken up selectively by the tissue. In cases of local administration or selective uptake, the effective local concentration of the agent may not be related to plasma concentration.

9. Immunopotentiating Compositions

The invention also contemplates a composition, comprising an immunopotentiating agent selected from a polypeptide as described in Section 2, or a polynucleotide as described in Section 3, or a vector as described in Section 2.6, together with a pharmaceutically acceptable carrier. One or more immunopotentiating agents can be used as actives in the preparation of immunopotentiating compositions. Such preparation uses routine methods known to persons skilled in the art. Typically, such compositions are prepared as injectables, either as liquid solutions or suspensions; solid forms suitable for solution in, or suspension in, liquid prior to injection may also be prepared. The preparation may also be emulsified. The active immunogenic ingredients are often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, and/or adjuvants that enhance the effectiveness of the vaccine. Examples of adjuvants which may be effective include but are not limited to: aluminium hydroxide, N-acetyl-muramyl-L-threonyl-D-isoglutamine (thur-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 1983A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion. For example, the effectiveness of an adjuvant may be determined by measuring the amount of antibodies resulting from the administration of the composition, wherein those antibodies are directed against one or more antigens presented by the treated cells of the composition.

The immunopotentiating agents may be formulated into a composition as neutral or salt forms. Pharmaceutically acceptable salts include the acid addition salts (formed with free amino groups of the peptide) and which are formed with inorganic acids such as, for example, hydrochloric or phosphoric acids, or such organic acids such as acetic, oxalic, tartaric, maleic, and the like. Salts formed with the free carboxyl groups may also be derived from inorganic basis such as, for example, sodium, potassium, ammonium, calcium, or ferric hydroxides, and such organic basis as isopropylamine, trimethylamine, 2-ethylamino ethanol, histidine, procaine, and the like.

If desired, devices or compositions containing the immunopotentiating agents suitable for sustained or intermittent release could be, in effect, implanted in the body or topically applied thereto for the relatively slow release of such materials into the body.

The compositions are conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1%-2%. Oral formulations include such normally employed excipients as, for example, pharmaceutical grades of mannitol, lactose, starch, magnesium carbonate, and the like. These compositions take the form of solutions, suspensions, tablets, pills, capsules, sustained release formulations or powders and contain 10%-95% of active ingredient, preferably 25%-70%.

If desired, the composition may be in the form of a composition of matter for eliciting a humoral and a cellular immune response against a target antigen, comprising antigen-presenting cells which express a processed form of a polypeptide as described in Section 2 for presentation to, and modulation of, T cells. Antigen-primed antigen-presenting cells may be prepared by a method including contacting antigen-presenting cells with a polypeptide as described in Section 2 for a time and under conditions sufficient to permit said polypeptide to be internalised by the antigen-presenting cells; and culturing the polypeptide-containing antigen-presenting cells for a time and under conditions sufficient for the polypeptide to be processed for presentation by the antigen-presenting cells. The antigen-presenting cells may be selected from dendritic cells, macrophages and B cells. In preferred embodiments of the invention, the antigen-presenting cells are dendritic cells.

With regard to nucleic acid based compositions, all modes of delivery of such compositions are contemplated by the present invention. Delivery of these compositions to cells or tissues of an animal may be facilitated by microprojectile bombardment, liposome mediated transfection (e.g., lipofectin or lipofectamine), electroporation, calcium phosphate or DEAE-dextran-mediated transfection, for example. In an alternate embodiment, a synthetic construct may be used as a therapeutic or prophylactic composition in the form of a “naked DNA” composition as is known in the art. A discussion of suitable delivery methods may be found in Chapter 9 of CURRENT PROTOCOLS IN MOLECULAR BIOLOGY (Eds. Ausubel et al.; John Wiley & Sons Inc., 1997 Edition) or on the Internet site DNAvaccine.com. The compositions may be administered by intradermal (e.g., using panjet™ delivery) or intramuscular routes.

The step of introducing a nucleic acid based composition (e.g., an expression vector) into a target cell will differ depending on the intended use and species, and can involve one or more of non-viral and viral vectors, cationic liposomes, retroviruses, and adenoviruses such as, for example, described in Mulligan, R. C., (1993 Science 260 926-932) which is hereby incorporated by reference. Such methods can include, for example:

A. Local application of the synthetic polynucleotide by injection (Wolff et al., 1990, Science 247 1465-1468, which is hereby incorporated by reference), surgical implantation, instillation or any other means. This method can also be used in combination with local application by injection, surgical implantation, instillation or any other means, of cells responsive to the protein encoded by the synthetic polynucleotide so as to increase the effectiveness of that treatment. This method can also be used in combination with local application by injection, surgical implantation, instillation or any other means, of another factor or factors required for the activity of said protein.

B. General systemic delivery by injection of DNA, (Calabretta et al., 1993, Cancer Treat. Rev. 19 169-179, which is incorporated herein by reference), or RNA, alone or in combination with liposomes (Zhu et al., 1993, Science 261 209-212, which is incorporated herein by reference), viral capsids or nanoparticles (Bertling et al., 1991, Biotech. Appl. Biochem. 13 390-405, which is incorporated herein by reference) or any other mediator of delivery. Improved targeting might be achieved by linking the synthetic polynucleotide to a targeting molecule (the so-called “magic bullet” approach employing, for example, an antibody), or by local application by injection, surgical implantation or any other means, of another factor or factors required for the activity of the protein encoding said synthetic polynucleotide, or of cells responsive to said protein.

C. Injection or implantation or delivery by any means, of cells that have been modified ex vivo by transfection (for example, in the presence of calcium phosphate: Chen et al., 1987, Mole. Cell Biochem. 7 2745-2752, or of cationic lipids and polyamines: Rose et al., 1991, BioTech. 10 520-525, which articles are incorporated herein by reference), infection, injection, electroporation (Shigekawa et al., 1988, BioTech. 6 742-751, which is incorporated herein by reference) or any other way so as to increase the expression of said synthetic polynucleotide in those cells. The modification can be mediated by plasmid, bacteriophage, cosmid, viral (such as adenoviral or retroviral; Mulligan, 1993, Science 260 926-932; Miller, 1992, Nature 357 455-460; Salmons et al., 1993, Hum. Gen. Ther. 4 129-141, which articles are incorporated herein by reference) or other vectors, or other agents of modification such as liposomes (Zhu et al., 1993, Science 261 209-212, which is incorporated herein by reference), viral capsids or nanoparticles (Bertling et al., 1991, Biotech. Appl. Biochem. 13 390-405, which is incorporated herein by reference), or any other mediator of modification. The use of cells as a delivery vehicle for genes or gene products has been described by Barr et al., 1991, Science 254 1507-1512 and by Dhawan et al., 1991, Science 254 1509-1512, which articles are incorporated herein by reference. Treated cells can be delivered in combination with any nutrient, growth factor, matrix or other agent that will promote their survival in the treated subject.

Also encapsulated by the present invention is a method for treatment and/or prophylaxis of a cancer or tumour, especially a cancer or tumour of the a cancer of the brain, testis or kidney, comprising administering to a patient in need of such treatment an effective amount of an immunopotentiating composition as broadly described above.

The immunopotentiating compositions or vaccines may be administered in a manner compatible with the dosage formulation, and in such amount as is therapeutically effective to alleviate patients from the cancer or tumour or as is prophylactically effective to prevent incidence of the cancer or tumour in the patient. The dose administered to the patient, in the context of the present invention, should be sufficient to effect a beneficial response in the patient over time such as an amelioration or reversal of the symptoms associated with cancer or tumour. The quantity of the composition or vaccine to be administered may depend on the subject to be treated inclusive of the age, sex, weight and general health condition thereof. In this regard, precise amounts of the composition or vaccine for administration will depend on the judgement of the practitioner. In determining the effective amount of the composition or vaccine to be administered in the treatment of a cancer or tumour, the physician may evaluate the progression of the cancer or the size of the tumour over time. In any event, those of skill in the art may readily determine suitable dosages of the composition or vaccine of the invention.

In a preferred embodiment, DNA-based immunopotentiating agent (e.g., 100 μg) is delivered intradermally into a patient at day 1 and at week 8 to prime the patient. A recombinant poxvirus (e.g., at 10⁷ pfu/mL) from which substantially the same immunopotentiating agent can be expressed is then delivered intradermally as a booster at weeks 16 and 24, respectively.

The effectiveness of the immunisation may be assessed using any suitable technique. For example, CTL lysis assays may be employed using stimulated splenocytes or peripheral blood mononuclear cells (PBMC) on peptide coated or recombinant virus infected cells using ⁵¹Cr labelled target cells. Such assays can be performed using for example primate, mouse or human cells (Allen et al., 2000, J. Immunol. 164 (9): 4968-4978 also Woodberry et al., infra). Alternatively, the efficacy of the immunisation may be monitored using one or more techniques including, but not limited to, HLA class I Tetramer staining—of both fresh and stimulated PBMCs (see for example Allen et al., supra), proliferation assays (Allen et al., supra), Elispot™ Assays and intracellular INF-gamma staining (Allen et al., supra), ELISA Assays—for linear B cell responses; and Western blots of cell sample expressing the synthetic polynucleotides.

10. Genetically Modified Animals

The invention also provides genetically modified, non-human animals having an altered TTYH2 gene. Alterations to the TTYH2 gene include, but are not restricted to, deletions or other loss of function mutations, introduction of an exogenous gene having a nucleotide sequence with targeted or random mutations, introduction of an exogenous gene from another species, or a combination thereof. The genetically modified animal may be either homozygous or heterozygous for the alteration.

Useful sequences for producing the genetically modified animals of the invention include, but are not restricted to, open reading frames encoding specific polypeptides or domains, introns, and adjacent 5′ and 3′ non-coding nucleotide sequences involved in the regulation of expression, up to about 1 kb beyond the coding region, but possibly further in either direction. The DNA sequences encoding TTYH2 may be cDNA (e.g., SEQ ID NO: 1 or 6) or genomic DNA (e.g., SEQ ID NO: 4) or a fragment thereof. A genomic sequence of interest comprises the nucleic acid present between the initiation codon and the stop codon, as defined in the listed sequences, including all of the introns that are normally present in a native chromosome. It may further include the 3′ and 5′ untranslated regions found in the mature mRNA. It may further include specific transcriptional and translational regulatory sequences, such as promoters, enhancers, etc., including about 1 kb, but possibly more, of flanking genomic DNA at either the 5′ or 3′ end of the transcribed region. The genomic DNA may be isolated as a fragment of 100 kb or smaller; and substantially free of flanking chromosomal sequence. The sequence of this 5′ region, and further 5′ upstream sequences and 3′ downstream sequences, may be utilised for promoter elements, including enhancer binding sites, that provide for expression in cells where TTYH2 is expressed. The cell specific expression is useful for determining the pattern of expression, and for providing promoters that mimic the native pattern of expression. Naturally occurring polymorphisms in the promoter region are useful for determining natural variations in expression, particularly those that may be associated with disease. Alternatively, mutations may be introduced into the promoter region to determine the effect of altering expression in experimentally defined systems. Methods for the identification of specific DNA motifs involved in the binding of transcriptional factors are known in the art, e.g. sequence similarity to known binding motifs, gel retardation studies, etc. For examples, reference may be made to Blackwell et al. (1995, Mol Med 1: 194-205), Mortlock et al. (1996, Genome Res. 6: 327-33), and Joulin and Richard-Foy (1995, Eur J Biochem 232: 620-626). Further, there is recent evidence that expression of certain mRNA species can be regulated at the translational level so that protein expression is restricted to particular cells types. The key features of these mRNA species are multiple translational initiation sites in the 5′ region of the coding sequence and a long 3′ untranslated region that controls mRNA translation in part.

The regulatory sequences may be used to identify cis acting sequences required for transcriptional or translational regulation of TTYH2 expression, especially in different cells or stages of development or differentiation, and to identify cis acting sequences and trans acting factors that regulate or mediate expression. Such transcription or translational control regions may be operably linked to a TTYH2 gene in order to promote expression of wild type or altered TTYH2 or other proteins of interest in cultured cells, or in embryonic, foetal or adult tissues, and for gene therapy.

The polynucleotides used for the production of the genetically modified animal may encode all or a part of the TTYH2 polypeptides or domains thereof as appropriate. Fragments of the DNA sequence may be obtained by chemically synthesising oligonucleotides in accordance with conventional methods, by restriction enzyme digestion, by PCR amplification, etc. For the most part, DNA fragments will be of at least 15 nucleotides, usually at least 18 nucleotides, more usually at least about 50 nucleotides. Such small DNA fragments are useful as primers for PCR, hybridisation screening, etc. Larger DNA fragments, i.e. greater than 100 nucleotides are useful for production of the encoded polypeptide. For use in amplification reactions, such as PCR, a pair of primers will be used.

The genetically modified animals of the present invention typically, but not exclusively, comprise a foreign or exogenous polynucleotide sequence or transgene present as an extrachromosomal element or stably integrated in all or a portion of its cells, especially in germ cells. Unless otherwise indicated, it will be assumed that a genetically modified animal comprises stable changes to the germline sequence. During the initial construction of the animal, “chimeras” or “chimeric animals” are generated, in which only a subset of cells have the altered genome. Chimeras are primarily used for breeding purposes in order to generate the desired genetically modified animal. Animals having a heterozygous alteration are generated by breeding of chimeras. Male and female heterozygotes are typically bred to generate homozygous animals.

Genetically modified animals fall into two groups, colloquially termed “knockouts” and “knockins”. In the present invention, knockouts have a partial or complete loss of function in one or both alleles of the endogenous TTYH2 gene. Knockins have an introduced transgene (i.e., foreign gene) with altered genetic sequence and function from the endogenous gene. Increased (including ectopic) or decreased expression may be achieved by introduction of an additional copy of the target gene, or by operatively inserting a regulatory sequence that provides for enhanced expression of an endogenous copy of the target gene. These changes may be constitutive or conditional, i.e. dependent on the presence of an activator or repressor. The foreign gene is usually either from a different species than the animal host, or is otherwise altered in its coding or non-coding sequence. The introduced gene may be a wild-type gene, naturally occurring polymorphism, or a genetically manipulated sequence, for example having deletions, substitutions or insertions in the coding or non-coding regions. The introduced sequence may encode a TTYH2 polypeptide, or may utilise the TTYH2 promoter operably linked to a reporter gene. Where the introduced gene is a coding sequence, it is usually operably linked to a promoter, which may be constitutive or inducible, and other regulatory sequences required for expression in the host animal. A knockin and a knockout may be combined, such that the naturally occurring gene is disabled, and an altered form introduced.

Preferably, a genetically modified animal of the invention has a partial or complete loss of function in one or both alleles of the endogenous TTYH2 gene and thus falls into the “knockout” group of genetically modified animals. A knockout may be achieved by a variety of mechanisms, including introduction of a disruption of the coding sequence, e.g. insertion of one or more stop codons, insertion of a DNA fragment, etc., deletion of coding sequence, substitution of stop codons for coding sequence, etc. In some cases the foreign transgene sequences are ultimately deleted from the genome, leaving a net change to the native sequence. Different approaches may be used to achieve the “knockout”. A chromosomal deletion of all or part of the native TTYH2 may be induced, including deletions of the non-coding regions, particularly the promoter region, 3′ regulatory sequences, enhancers, or deletion of a gene that activates expression of TTYH2. A functional knockout may also be achieved by the introduction of an anti-sense construct that blocks expression of the native TTYH2 genes (for example, see Li and Cohen, 1996, Cell 85: 319-329). “Knockouts” also include conditional knock-outs, for example where alteration of the target gene occurs upon exposure of the animal to a substance that promotes target gene alteration, introduction of an enzyme that promotes recombination at the target gene site (e.g. Cre in the Cre-lox system), or other method for directing the target gene alteration postnatally.

In a preferred embodiment, the partial or complete loss of function in one or both alleles of the TTYH2 gene is effected by disruption of that gene. Accordingly, the genetically modified animal preferably comprises a disruption in at least one allele of the endogenous TTYH2 gene. In accordance with the present invention, the disruption suitably results in an inability of said animal to produce a corresponding functional expression product or detectable levels of said expression product. Accordingly, a disruption in said endogenous TTYH2 gene may result in a reduced level and/or functional activity of TTYH2 or in an inability of said animal to produce a functional TTYH2 or detectable levels of TTYH2 relative to a corresponding animal without said disruption.

A disruption typically comprises an insertion of a nucleic acid sequence into one region of the native genomic sequence (usually one or more exons) and/or the promoter region of a gene so as to decrease or prevent expression of that gene in the cell as compared to the wild-type or naturally occurring sequence of the gene. By way of example, a nucleic acid construct can be prepared containing a selectable marker gene which is inserted into a targeting nucleic acid sequence that is complementary to a genomic sequence (promoter and/or coding region) to be disrupted. Useful genomic sequences to be disrupted include, but are not restricted to, TTYH12 open reading frames encoding polypeptides or domains, introns, and adjacent 5′ and 3′ non-coding nucleotide sequences involved in regulation of gene expression. Accordingly, a targeting sequence may comprise some or part of the nucleic acid present between the initiation codon and the stop codon, as defined in the listed sequences, including some or all of the introns that are normally present in a native chromosome. It may further include the 3′ and 5′ untranslated regions found in the mature mRNA. It may further include specific transcriptional and translational regulatory sequences, such as promoters, enhancers, etc., including about 1 kb, but possibly more, of flanking genomic DNA at either the 5′ or 3′ end of the transcribed region. When the nucleic acid construct is then transfected into a cell, the construct will integrate into the genomic DNA. Thus, many progeny of the cell will no longer express the gene at least in some cells, or will express it at a decreased level, as the genomic sequence is now disrupted by the selection marker.

In another embodiment, an individual disruption reduces, abrogates or otherwise impairs the expression of a TTYH2 gene and in this regard, the disruption may reside in the deletion of at least a portion of the transcriptional and/or translational regulatory sequences associated with said TTYH2 gene.

Specific examples of the genetically modified animals of the present invention include those containing:
(a) a substantially complete loss of function in a single allele of the endogenous TTYH2 gene (i.e., TTYH2^{+/-});
(b) a substantially complete loss of function in both alleles of the endogenous TTYH2 gene (i.e., TTYH2^{-/-}); or
(c) genetic or functional equivalents of (a) or (b).

Suitable genetic or functional equivalent animals include those containing anti-sense constructs comprising a sequence complementary to at least a portion of an endogenous TTYH2 gene which will block expression of a corresponding expression product to a level analogous to that in (a) or (b) above. It should be understood that any and all such equivalents are contemplated to fall within the scope of the present invention.

Non-human animals for genetic modification include, but are not restricted to, vertebrates, preferably mammals such as rodents, non-human primates, ovines, bovines, ruminants, lagomorphs, porcines, caprines, equines, canines, felines, aves, etc. In a preferred embodiment, the non-human animal is selected from the order Rodentia, which includes rodents i.e., placental mammals (class Euthria) which include the family Muridae (rats and mice). In a particularly preferred embodiment, the non-human animal is a mouse.

The genetically modified animals of the invention are suitably produced using a vector, which is preferably but not exclusively a targeting construct, comprising a polynucleotide of the invention or biologically active fragment thereof or variant or derivative of these. Specific constructs or vectors of interest include, but are not limited to, anti-sense TTYH2 constructs comprising a sequence complementary to a polynucleotide, fragment, variant or derivative as herein described, which will block native TTYH2 expression, expression of dominant negative TTYH2 mutations, and over-expression of a TTYH2 gene.

A detectable marker, such as lacZ, or a selection marker, such as neo, may be introduced into the locus, where upregulation of expression will result in an easily detected change in phenotype. Vectors utilising the TTYH2 promoter region, in combination with a reporter gene or with the coding region are also of interest.

A series of small deletions and/or substitutions may be made in the TTYH2 gene to determine the role of different exons in DNA binding, transcriptional regulation, etc. By providing expression of TTYH2 protein in cells in which it is otherwise not normally produced, one can induce changes in cell behaviour.

A gene disruption resulting in partial or complete loss of function in one or both alleles of TTYH2 is suitably effected using a targeting construct or vector. Any polynucleotide sequence capable of disrupting an endogenous gene of interest (e.g., by introducing a premature stop codon, causing a frameshift mutation, disrupting proper splicing, etc.) may be employed in this regard. In a preferred embodiment, the vector, or an ancillary vector, comprises a positive selectable marker gene (e.g., hyg or neo). The disruption may reduce or prevent the expression of TTYH2 or may render the resulting TTYH2 polypeptide completely non-functional. Reduced levels of TTYH2 refer to a level of TTYH2 which is lower than that found in a wild-type animal. The level of TTYH2 produced in an animal of interest may be determined by a variety of methods including Western blot analysis of protein extracted from suitable cell types including, but not restricted to, kidney cells, lymphocytes or melanocytes. A lack of ability to produce functional TTYH2 includes within its scope the production of undetectable levels of functional TTYH2 (e.g., by Western blot analysis). In contrast, a functional TTYH2 is a molecule which retains the biological activity of the wild-type TTYH2 and which preferably is of the same molecular weight as the wild-type molecule.

Targeting vectors for homologous recombination will comprise at least a portion of the TTYH2 gene with the desired genetic modification, and will include regions of homology to the target locus. Those regions may be non-isogenic, but are preferably isogenic, to the target locus. Conveniently, markers for positive and negative selection are included. Methods for generating cells having targeted gene modifications through homologous recombination are known in the art. Various techniques for transfecting animal and particularly mammalian cells are described for example by Keown et al. (1990, Methods in Enzymology 185: 527-537).

In a preferred embodiment, the targeting vector includes polynucleotide sequences comprising a selectable marker gene flanked on either side by TTYH2 gene sequences. The targeting vector will generally contain gene sequences sufficient to permit the homologous recombination of the targeting vector into at least one allele of the endogenous gene resident in the chromosomes of the target or recipient cell (e.g., ES cells). In a preferred embodiment, the cell employed is an ES cell from a mammal within the order Rodentia and most preferably a mouse ES cell. Typically, the targeting vector will contain approximately 1 to 15 kb of DNA homologous to the endogenous TTYH2 gene (more than 15 kb or less than 5 kb of the endogenous TTYH2 gene sequences may be employed so long as the amount employed is sufficient to permit homologous recombination into the endogenous gene); this 1 to 15 kb of DNA is preferably divided on each side of the selectable marker gene.

The targeting construct may contain more than one selectable marker gene. The selectable marker is preferably a polynucleotide which encodes an enzymatic activity that confers resistance to an antibiotic or drug upon the cell in which the selectable marker is expressed. Selectable markers may be “positive”; positive selectable markers typically are dominant selectable markers, i.e., genes which encode an enzymatic activity which can be detected in any animal, preferably mammalian, cell or cell line (including ES cells). Examples of dominant selectable markers include the bacterial aminoglycoside 3′ phosphotransferase gene (also referred to as the neo gene) which confers resistance to the drug G418 in animal cells, the bacterial hygromycin G phosphotransferase (hyg) gene which confers resistance to the antibiotic hygromycin and the bacterial xanthine-guanine phosphoribosyl transferase gene (also referred to as the gpt gene) which confers the ability to grow in the presence of mycophenolic acid. Selectable markers may be ‘negative’; negative selectable markers encode an enzymatic activity whose expression is cytotoxic to the cell when grown in an appropriate selective medium. For example, the HSV-tk gene is commonly used as a negative selectable marker. Expression of the HSV-tk gene in cells grown in the presence of gancyclovir or acyclovir is cytotoxic; thus, growth of cells in selective medium containing gancyclovir or acyclovir selects against cells capable of expressing a functional HSV TK enzyme.

When more than one selectable marker gene is employed, the targeting vector preferably contains a positive selectable marker (e.g. the neo gene) and a negative selectable marker (e.g., the Herpes simplex virus tk (HSV-tk) gene). The presence of the positive selectable marker permits the selection of recipient cells containing an integrated copy of the targeting vector whether this integration occurred at the target site or at a random site. The presence of the negative selectable marker permits the identification of recipient cells containing the targeting vector at the targeted site (i.e., which has integrated by virtue of homologous recombination into the target site); cells which survive when grown in medium which selects against the expression of the negative selectable marker do not contain a copy of the negative selectable marker.

Preferred targeting vectors of the present invention are of the “replacement-type”, wherein integration of a replacement-type vector results in the insertion of a selectable marker into the target gene. Replacement-type targeting vectors may be employed to disrupt a gene resulting in the generation of a null allele (i.e., an allele incapable of expressing a functional protein; null alleles may be generated by deleting a portion of the coding region, deleting the entire gene, introducing an insertion and/or a frameshift mutation, etc.) or may be used to introduce a modification (e.g., one or more point mutations) into a gene.

The genetically modified animals of the present invention are preferably generated by introduction of the above vectors into embryonal stem (ES) cells. ES cells are obtained by culturing pre-implantation embryos in vitro under appropriate conditions (Evans, et al., 1981, Nature 292: 154-156; Bradley, et al., 1984, Nature 309: 255-258; Gossler, et al., 1986, Proc. Natl. Acad. Sci. USA 83: 9065-9069; and Robertson, et al., 1986, Nature 322: 445-448). Transgenes can be efficiently introduced into the ES cells by DNA transfection using a variety of methods known to the art including electroporation, calcium phosphate co-precipitation, protoplast or spheroplast fusion, lipofection and DEAE-dextran-mediated transfection. Transgenes may also be introduced into ES cells by retrovirus-mediated transduction or by micro-injection. Cells are subsequently plated onto a feeder layer in an appropriate medium and those containing the transgene may be detected by employing a selective medium. Alternatively, PCR may be used to screen for ES cells which have integrated the transgene. After sufficient time for colonies to grow, they are picked and analysed for the occurrence of homologous recombination or integration of the vector. This PCR technique obviates the need for growth of the transfected ES cells under appropriate selective conditions prior to transfer into the blastocoel of a non-human animal. Those colonies that are positive may then be used for embryo manipulation and blastocyst injection. Blastocysts are obtained from 4 to 6 week old superovulated females. The ES cells are trypsinised, and the modified cells are injected into the blastocoel of the blastocyst. After injection, the blastocysts are returned to each uterine horn of pseudopregnant females. Females are then allowed to go to term and the resulting litters screened for mutant cells having the vector. By providing for a different phenotype of the blastocyst and the ES cells, chimeric progeny can be readily detected. For a review, see Jaenisch (1988, Science 240: 1468-1474). The chimeric progeny are screened for the presence of the transgene and males and females having the transgene are mated to produce homozygous progeny. If the gene alterations cause lethality at some point in development, tissues or organs can be maintained as allogeneic or congenic grafts or transplants, or in in vitro culture.

Alternative methods for the generation of genetically modified animals are known to those skilled in the art. For example, embryonal cells at various developmental stages can be used to introduce transgenes for the production of genetically modified animals. Different methods are used depending on the stage of development of the embryonal cell. The zygote, particularly at the pronucleal stage (i.e., prior to fusion of the male and female pronuclei), is a preferred target for micro-injection. The use of zygotes as a target for gene transfer has a major advantage in that in most cases the injected DNA will be incorporated into the host genome before the first cleavage (Brinster, et al., 1985, Proc. Natl. Acad. Sci. USA 82: 4438-4442). As a consequence, all cells of the genetically modified non-human animal will carry the incorporated transgene. This will in general also be reflected in the efficient transmission of the transgene to offspring of the founder since 50% of the germ cells will harbour the transgene. U.S. Pat. No. 4,873,191 describes a method for the micro-injection of zygotes.

Retroviral infection can also be used to introduce transgenes into a non-human animal. The developing non-human embryo can be cultured in vitro to the blastocyst stage. During this time, the blastomeres can be targets for retroviral infection (Janenich, 1976, Proc. Natl. Acad. Sci. USA 73: 1260-1264). Retroviral infection is easily and efficiently obtained by culturing the blastomeres on a monolayer of virus-producing cells (Van der Putten, supra; Stewart, et al., 1987, EMBO J. 6: 383-388). Alternatively, infection can be performed at a later stage. Virus or virus-producing cells can be injected into the blastocoel (Jahner, D. et al., 1982, Nature 298: 623-628). It is also possible to introduce transgenes into the germline, albeit with low efficiency, by intrauterine retroviral infection of the midgestation embryo (Jahner, D. et al., 1982, supra). An additional means of using retroviruses or retroviral vectors to create genetically modified animals known to the art involves the micro-injection of retroviral particles or mitomycin C-treated cells producing retrovirus into the perivitelline space of fertilised eggs or early embryos (PCT International Application Publication No. WO 90/08832) and Haskell and Bowen, 1995, Mol. Reprod. Dev. 40: 386).

In selecting lines of an animal species to work the present invention, they may be selected for criteria such as embryo yield, pronuclear visibility in the embryos, reproductive fitness, colour selection of genetically modified offspring or availability of ES cell clones. For example, if genetically modified mice are to be produced, lines such as C57BL/6 may be preferred.

The age of the animals that are used to obtain embryos and to serve as surrogate hosts is a function of the species used. When mice are used, for example, pre-puberal females are preferred as they yield more embryos and respond better to hormone injections. In this regard, administration of hormones or other chemical compounds may be necessary to prepare the female for egg production, mating and/or implantation of embryos.

Genetically modified offspring of a surrogate host may be screened for the presence of the transgene by any suitable method. Screening may be accomplished by Southern or northern analysis using a probe that is complementary to at least a portion of the transgene or by PCR using primers complementary to portions of the transgene. Western blot analysis using an antibody against the protein encoded by the transgene may be employed as an alternative or additional method for screening. Alternative or additional methods for evaluating the presence of the transgene include without limitation suitable biochemical assays such as enzyme and/or immunological assays, histological stains for particular markers or enzyme activities and the like.

Progeny of the genetically modified mammals may be obtained by mating the genetically modified animal with a suitable partner or by in vitro fertilisation using eggs and/or sperm obtained from the genetically modified animal. Where in vitro fertilisation is used, the fertilised embryo is implanted into a surrogate host or incubated in vitro or both. Where mating is used to produce genetically modified progeny, the genetically modified animal may be back-crossed to a parental line, otherwise inbred or cross-bred with animals possessing other desirable genetic characteristics. The progeny may be evaluated for the presence of the transgene using methods described above, or other appropriate methods.

Genetically modified animals comprising genetic alterations resulting in partial or complete loss of function in one or both alleles of TTYH2 find a number of uses. For example, TTYH2 knockout mice provide a means for screening test compounds beneficial for modulating TTYH2 function. In addition, these animals provide a means for screening compounds for the treatment or prevention in patients of conditions associated with aberrant TTYH2 expression, especially cancers and tumours. In a particular preferred embodiment, these animals provide a means for screening compounds for therapeutic use in patients, which are useful inter alia in modulating tumorigenesis and especially for treating and/or preventing cancers or tumours. Thus, the invention also contemplates a process for screening a candidate agent for the ability to specifically modulate TTYH2 function. The process comprises administering a candidate agent to a genetically modified animal as broadly described above and to a corresponding wild-type animal, which is preferably a species or strain of animal from which the genetically modified animal was derived. The individual responses of the genetically modified animal and of the wild-type animal are then compared. A candidate agent tests positive as a specific modulator of TTYH2 function if there is a substantial modulation of the response under test in the wild-type animal but there is no substantial modulation of the tested response in the genetically modified animal.

In order that the invention may be readily understood and put into practical effect, particular preferred embodiments will now be described by way of the following non-limiting examples.

### EXAMPLES

### Example 1

Clinical Samples

Kidney tissue was collected at the time of nephrectomy from patients with RCC at the Princess Alexandra Hospital, Brisbane, Australia and stored at −80° C. Histological assessment of tumour tissue from the 16 patients used in this study confirmed clear cell RCC using the Heidelberg classification of renal cell tumours (Kovacs et al., 1997) with a tumour stage of T₁ or T₂, N₀, M₀ (early stage) as determined by the tumour, nodes, metastasis (TNM) staging of RCC (Guinan et al., 1997). Informed consent was obtained in all cases. Ethics approval was obtained from both the Queensland University of Technology and Princess Alexandra Hospital Ethics Committees.

### Example 2

Identification and Characterisation of TTYH2 cDNA and Protein

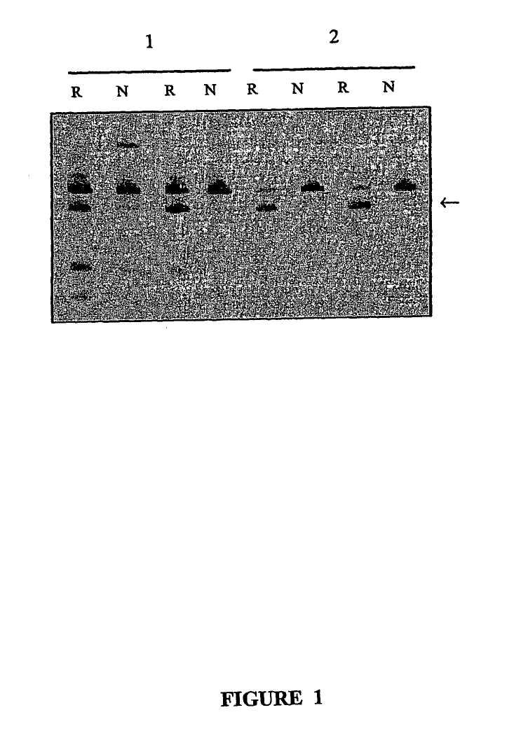

DD-PCR was performed using a Delta Differential Display kit (Clontech) according to the manufacturer's protocol with modifications as described previously (Bentley & Bassam, 1996; Rae et al., 2000). Duplicate paired RCC and normal kidney samples from 4 patients were analysed using 65 different primer combinations. A 235 bp fragment was identified as being up-regulated in 4 RCC samples when compared with matched normal kidney parenchyma (the DD-PCR results from 2 patients are shown FIG. 1). The 235 bp fragment was cloned into pGEM-T easy (Promega) and sequenced. This sequence was then used to screen the National Centre for Biotechnology Information (NCBI) GenBank non-redundant (nr) and human and mouse expressed sequence tag (EST) databases. Matching human EST clones (accession nos. H09521, AI623520, AA789226 and BE410734) and mouse clone (accession no. AI587692) were purchased (Incyte Genomics) and sequenced.

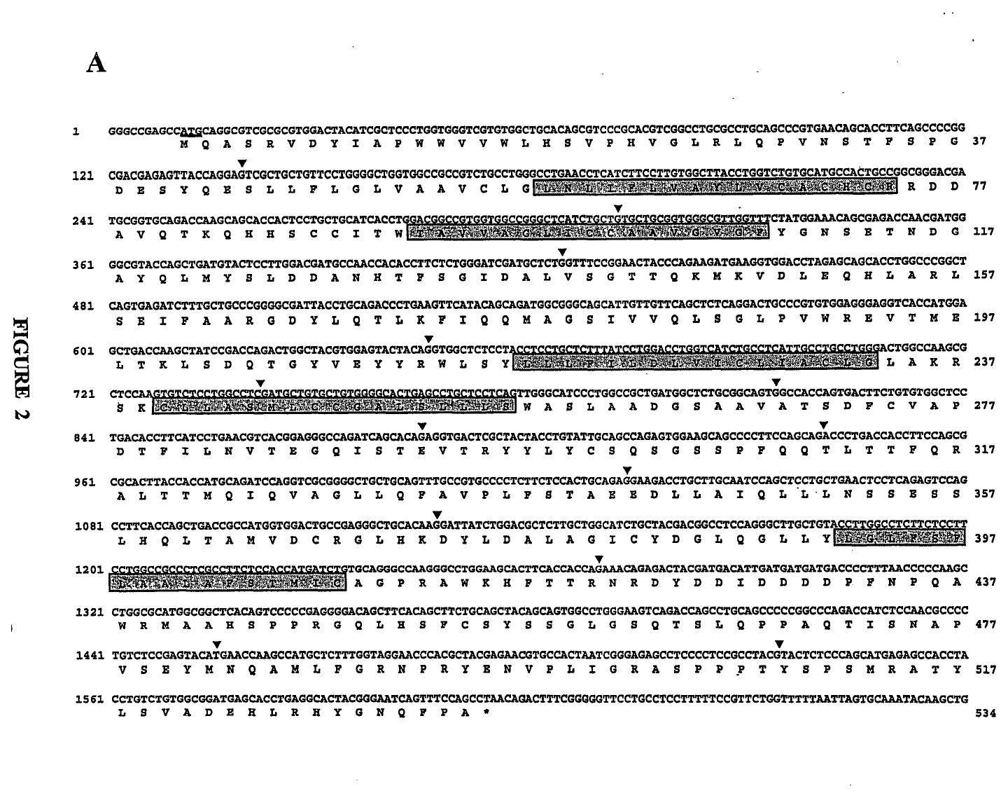

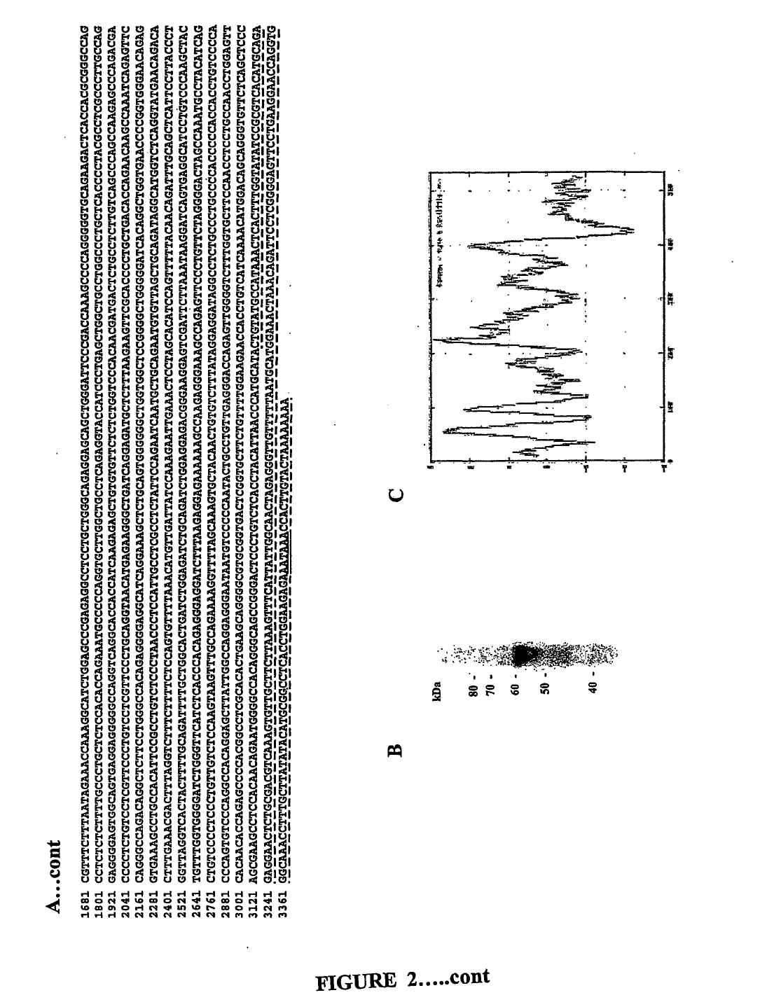

The 235 bp sequence showed 99% identity to two partial sequences contained within the GenBank nr database (accession nos. D63134 and D63135). Additional sequence was obtained from clones identified by searching the EST database. The complete contig (nucleotides 1-3420) (FIG. 2A) was obtained from human EST clones BE410734 (nucleotides 1-670), AA789226 (nucleotides 664-1725) and AI623520 (nucleotides 980-3420) and submitted to GenBank under the accession no. AF319952. The complete cDNA of 3420 bp [SEQ ID NO: 1] contains an open reading frame of 1602 bp (nucleotides 11-1612 of SEQ ID NO: 1) and a 3′ untranslated region (UTR) of 1808 bp. A consensus polyadenylation signal (AATAAA) is located at nucleotide 3404 (FIG. 2A). Nucleotides 358-401, 661-748 and 1309-1346′ of this novel cDNA showed significant homology (86%) to the human and mouse tweety homologue 1 TTYH1) genes. Based on the similarity to the TTYH1 genes at the nucleotide and protein (discussed below) levels, the inventors designated this novel gene human tweety homologue 2 (TTYH2; HGMW-approved symbol).

The orthologous mouse cDNA, Ttyh2, was identified from clones obtained by searching the mouse EST and high-throughput genomic sequence databases. The complete coding region (data not shown) was obtained from EST clone BF232787, the unordered genomic clone RP23-273C14 and EST clone AI587692. At the nucleotide level TTYH2 and Ttyh2 share 84% sequence identity. The Ttyh2 sequence has been submitted to GenBank under the accession no. AF329682 [SEQ ID NO: 6].

Translation from the most 5′ start codon of the TTYH2 cDNA predicted a 534 amino acid of 59.3 kDa. To determine whether this translation start site was functional a PCR product containing nucleotides 3-1618 of the TTYH2 cDNA was transcribed and translated in vitro. Briefly, A template for use in in vitro transcription/translation experiments, containing nucleotides 3 to 1618 of the TTYH2 cDNA, including the complete coding region, was generated by RT-PCR from RCC total RNA using a forward primer that contained the T7 RNA polymerase binding site 5′-GGATCCTAATACGACTCACTATAGGGAGACCACATGCCGAGCCATGCAGGCGT CGC-3′ [SEQ ID NO: 9] and the reverse primer 5′-CTGTTAGGCTGGAAACTGATTCCCG-3′ [SEQ ID NO: 10]. The fidelity of the PCR product was confirmed by sequencing. The PCR product (500 ng) was then transcribed and translated in vitro using a TNT T7 Coupled Reticulocyte Lysate system (Promega) in the presence of [³⁵S]methionine (Amersham). Control reactions were performed using a supplied luciferase cDNA and no DNA. Protein products were separated by electrophoresis on a 12% polyacrylamide gel (Biorad). The gel was fixed, washed in Amplify Reagent (Amersham), dried and exposed to X-ray film (AGFA Curix) for 5 hours at −80° C. Using the aforementioned procedure, a single protein of approximately 59 kDa was generated (FIG. 2B) indicating that the ATG codon at nucleotide 11-13 is capable of functioning as an initiating methionine in vitro.

To gain an insight into the potential role of TTYH2, the deduced protein sequence [SEQ ID NO: 2 and 5] was analysed for cellular sorting signals and functional and structural domains. This analysis indicated that TTYH2 lacks consensus signals for both secretion and translocation to the nucleus. However, five hydrophobic regions were identified spanning amino acids 58-74, 92-108, 217-233, 240-256 and 392-408 (FIGS. 2A and 2C), indicating that TTYH2 is a putative transmembrane protein. Using the PRED-TMR2 algorithm (http://o2.db.uoa.gr/PRED-TMR2), the orientation of TTYH2 is predicted to have the N-terminus located extracellularly and the C-terminus located intracellularly. A search of the PROSITE database (http:/www.expasy.ch) showed that the deduced TTYH2 protein contains one putative casein kinase II (residue 519) and two potential protein kinase C (residues 418 and 512) phosphorylation sites along with four consensus motifs for N-linked glycosylation (NXT/S) (residues 31, 129, 283 and 352) and five potential N-myristoylation sites (residues 49, 97, 110, 287 and 448). An RGD consensus sequence was also identified (residues 164-166) in a putative extracellular region of TTYH2. In other proteins, RGD motifs mediate binding to integrins thereby facilitating cell adhesion/de-adhesion events (D'Souza et al., 1991). Not wishing to be bound by any one particular theory or mode of operation, it is possible that TTYH2 has a role as a cell surface receptor mediating the binding of integrins.

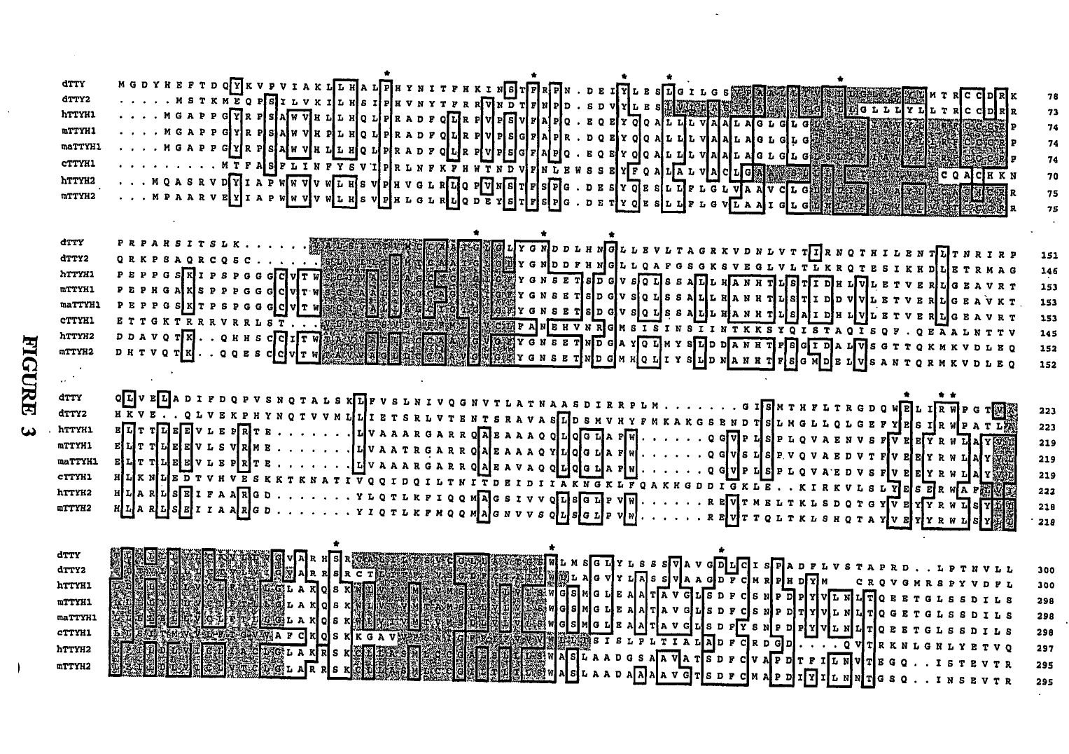

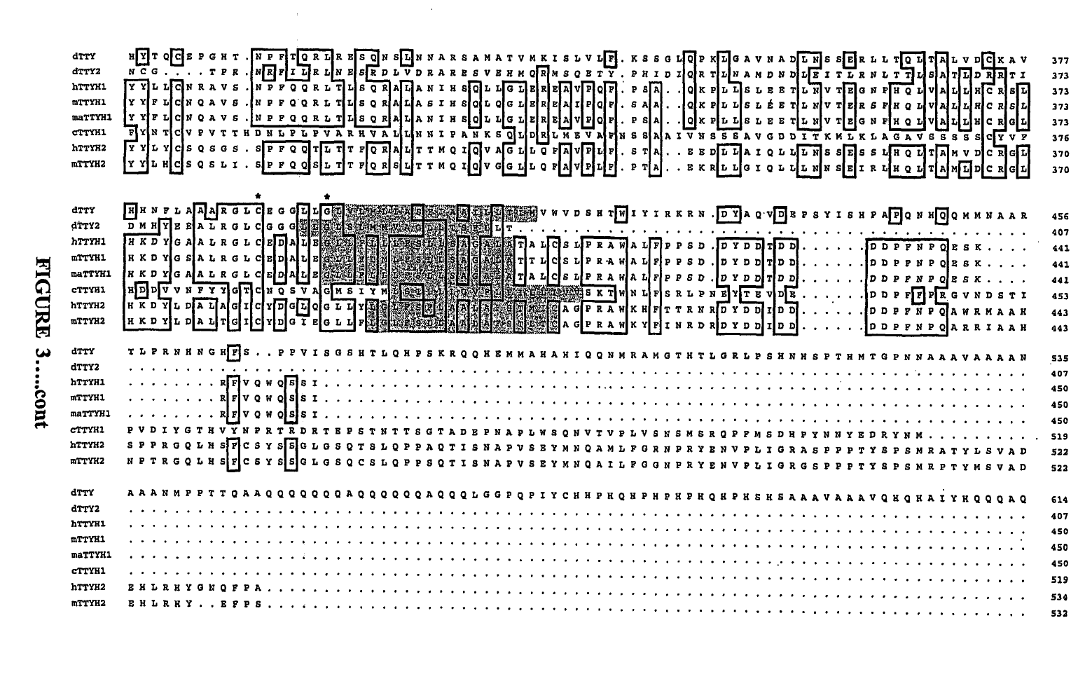

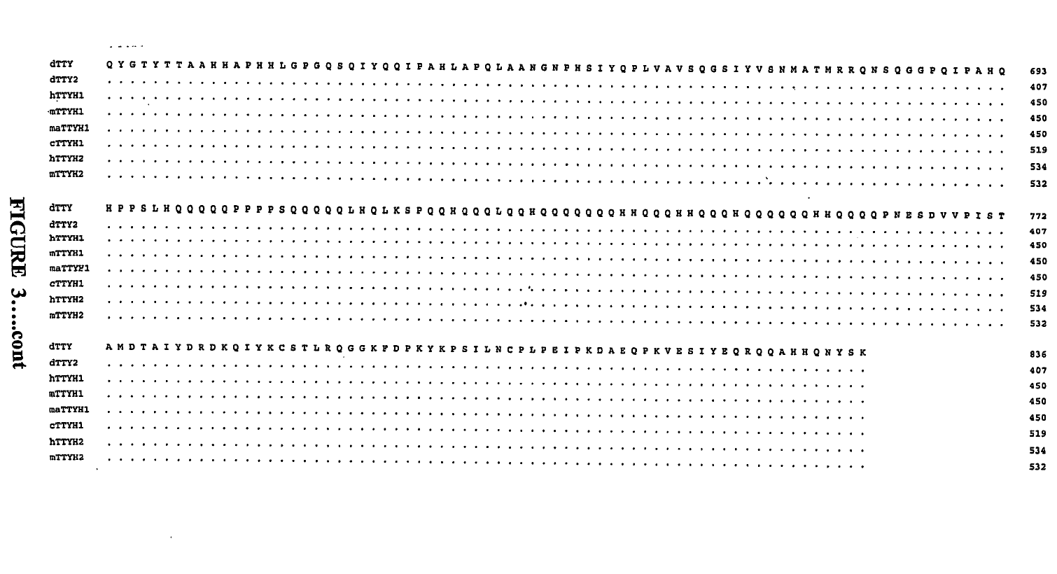

Human TTYH2 and mouse Ttyh2 were aligned against the six other members of the tweety-related protein family (FIG. 3). The alignment revealed that TTYH2 and Ttyh2 share 81% identity (89% similarity) and that the TTYH2 protein shows significant homology to human (43% identity, 63% similarity) and mouse (43% identity, 64% similarity) TTYH1 and Drosophila melanogaster tweety (28% identity, 46% similarity). Caenorhabditis elegans and macaque homologues for TTYH1 have also been identified (Campbell, 2000) and show 18% and 43% identity to TTYH2 respectively. Furthermore, a Drosophila melanogaster genomic clone (accession number AL035331) encodes a 407 amino acids of a second tweety-related protein, although this sequence is truncated by the end of the clone. The two Drosophila sequences share 42% identity (65% similarity). The eight tweety-related proteins share 16 residues. In addition to a high degree of sequence identity, the 5 putative transmembrane regions of the tweety-related proteins are located in almost identical positions. The arrangement of these transmembrane regions, referred to as the 2-2-1 arrangement (Campbell et al., 2000), consists of a pair of transmembrane regions near the N-terminus followed by a hydrophilic region of approximately 120 amino acids and another pair of transmembrane regions. There is then a further hydrophilic region of 120 amino acids followed by a single transmembrane region. The highest level of sequence variation between these proteins is seen in the C-terminus which is predicted to be located intracellularly. TTYH2 has a C-terminal extension of 84 amino acids relative to the human and mouse TTYH1. The Drosophila melanogaster tweety protein is 436 amino acids longer that TTYH2 as it contains a repetitive, hydrophilic C-terminal extension that shows no significant homology to other known proteins. It is likely that these putative intracellular C-terminal regions confer specificity of function of the tweety-related proteins.

### Example 3

Genomic Mapping and Gene Structure of TTYH2

A probe, generated from EST clone H09521 (nucleotides 1733-3420) plasmid DNA by nick-translation incorporating biotin-14-dATP, was hybridised in situ at a final concentration of 20 ng/mL to metaphases from two normal males. The fluorescence in situ hybridisation (FISH) method was modified from that previously described (Callen et al., 1990) in that chromosomes were stained before analysis with both 4′, 6-diamidialo-2-phenylindole (DAPI) (for chromosome identification) and propidium iodide (as counterstain). Images of metaphase preparations were captured by a cooled CCD camera using the ChromoScan™ image collection and enhancement system (Applied Imaging Corporation). FISH signals and the DAPI banding pattern were merged for figure preparation. To determine exon/intron splice sites and intron sizes the TTYH2 cDNA sequence was compared with the unordered fragments of genomic clone RP11-647F2 (accession no. AC021977). PCR was performed on genomic clone 2514K5 (accession no. AQ279008) (Research Genetics) cosmid DNA with specific primers (Table 1) to determine the approximate size of the TTYH2 introns not fully contained within clone RP11-647F2. The 25 mL PCR contained 300 ng clone 2514K5 DNA, 100 ng each primer, 5 mL each of PCR Life Technologies buffer A (60 mM Tris-SO₄, 18 mM (NH₄)₂SO₄, 1 mM MgSO₄) and B (60 mM Tris-SO₄, 18 mM (NH₄)₂SO₄, 2 mM MgSO₄), 0.4 mM dNTPs and 1U Elongase Taq polymerase (Life Technologies). Cycling parameters were 94° C. for 30 sec, 60-68° C. (Table 1) for 1 min and 72° C. for 5 min for 30 cycles followed by a further 7 mins extension at 72° C.

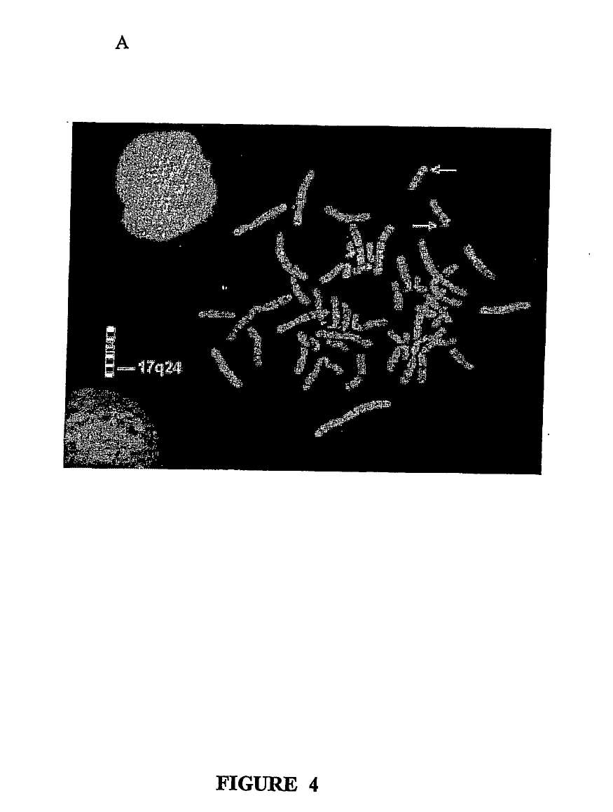

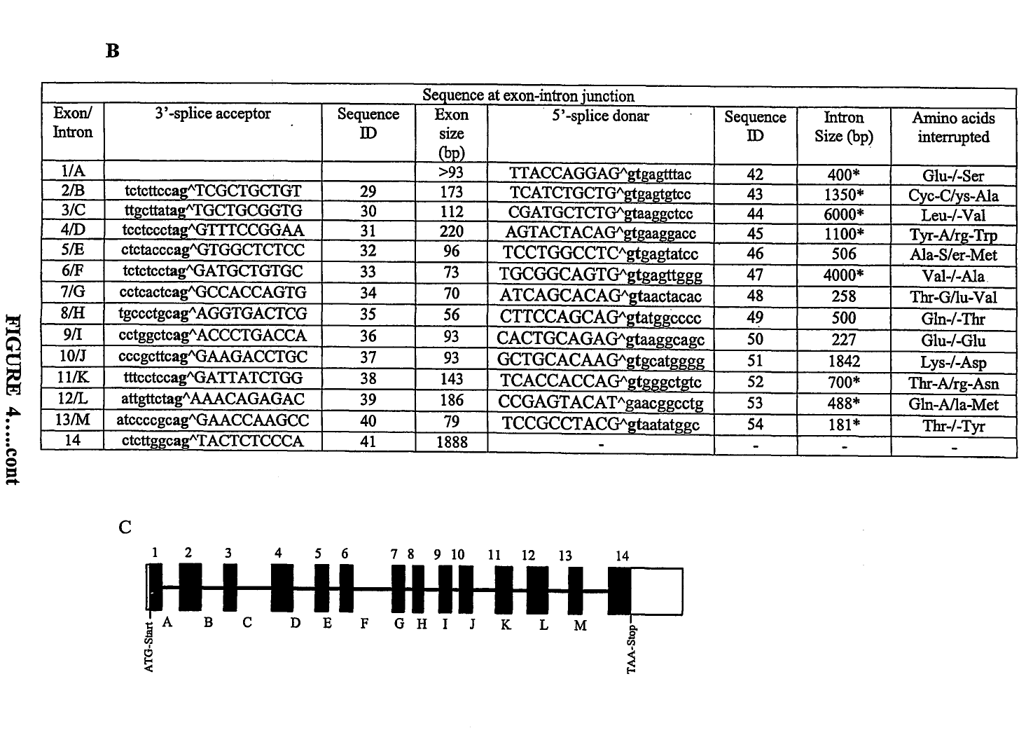

Using the above procedure, the chromosomal location of TTYH2 was mapped to human metaphase chromosomes from two normal males. Twenty metaphases from the first normal male were examined for fluorescent signal. All of these metaphases showed signal on one or both chromatids of chromosome 17 in the region 17q23-17q25; 70% of this signal was at 17q24 (FIG. 4A). There was a total of 10 non-specific background dots observed in these 20 metaphases. A similar result was obtained from hybridisation of the probe to 10 metaphases from the second normal male (data not shown). The mouse orthologue, Ttyh2, was identified on mouse genomic clone, RP23-273C14. This genomic clone has been localised to mouse chromosome 11, which contains regions syntenic to human chromosome 17q24.

The TTYH2 gene sequence is contained within genomic clones, RP11-647F2 (accession no. AC21977) and 2514K5 (accession no. AQ279008) identified by screening the GenBank high-throughput genome sequences and genome survey sequence databases respectively. RP11-647F2 is located on chromosome 17 between microsatellite markers D17S1807 and D17S1163, further refining the location of the TTYH2 gene. Intron/exon junctions and the size of introns E, G, H, I and J were determined by comparison of sequence of the TTYH2 cDNA and genomic clone RP11-647F2. To determine the sizes of the introns (A, B, C, D, F, K, L and M) not fully contained within the unordered fragments of RP11-647F2, PCR was performed on genomic clone 2514K5. The TTYH2 gene contains 14 exons and 13 introns. Exons ranged in size from 56 to 1888 bp and introns from 181 to >6000 bp (FIG. 4B). All of the intron/exon junctions conform to the GT-AG rule except for intron L which begins with GA instead of GT. A schematic representation of the TTYH2 gene is shown in FIG. 4C.

### Example 4

TTYH2 Expression Pattern

Northern blot analysis was carried out on 16 normal human tissues using a cRNA probe generated from TTYH2 cDNA. Briefly EST clone AI623520 (nucleotides 980-3420) plasmid DNA was linearised with SalI followed by transcription with T7 RNA polymerase to generate an antisense ³²P-UTP (Geneworks) labelled cRNA probe using a StripEz™ kit (Ambion). Hybridisation to Human Multiple Tissue Northern blots and a Mutiple Tissue Expression™ array (Clontech) was performed overnight at 68° C. in Ultrahyb™ hybridisation buffer (Ambion). The blots were then washed to a final stringency of 0.1×SSC/0.1% SDS at 71° C. and signals were detected by exposure to X-ray film (AGFA Curix). Blots were reprobed with ³²P-ATP random labelled (Ambion) b-actin cDNA probe to confirm RNA loadings.

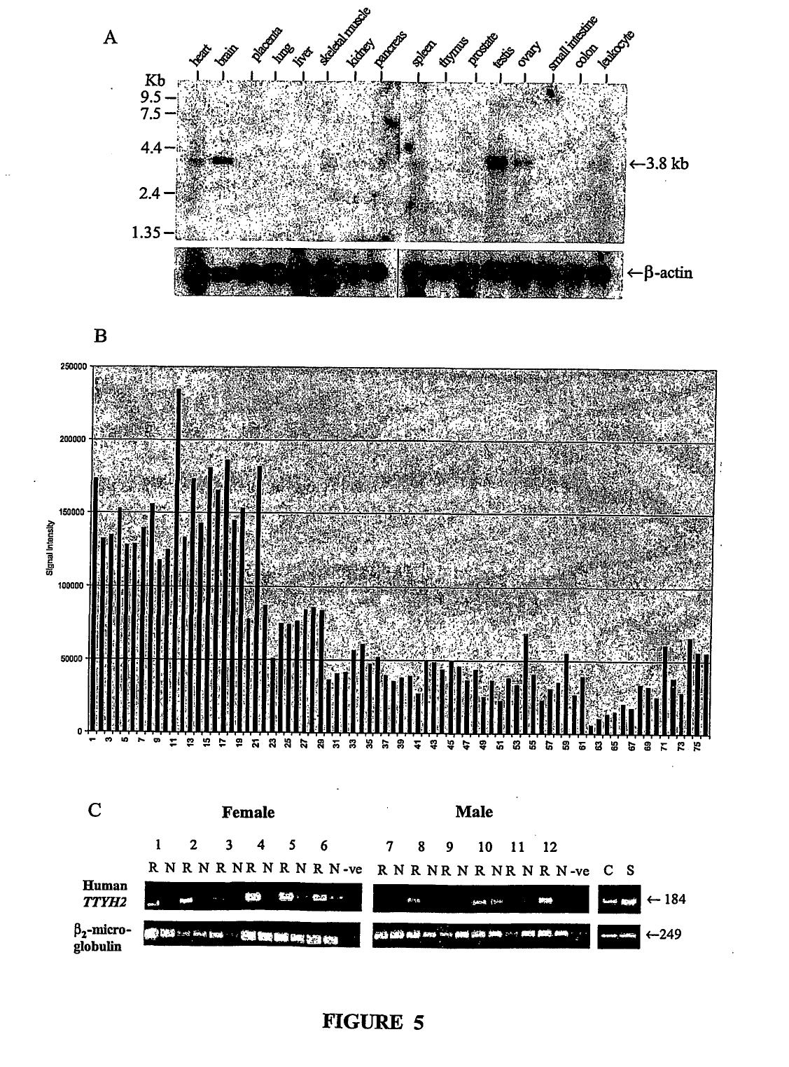

The above analysis revealed a transcript of 3.8 kb which was highly expressed in brain and testis. Lower levels were observed in the ovary and heart and very low expression in skeletal muscle, spleen and peripheral blood leucocytes (FIG. 5A). As the TTYH2 cDNA (3420 bp) together with a poly adenylation tail is shorter than the 3.8 kb transcript observed by Northern analysis, it is likely that there is additional 5′ UTR sequence still to be determined. To extend the expression analysis, a Multiple Tissue Expression array containing poly A+RNA from 76 normal human tissues and cell lines was probed with the TTYH2 cRNA probe. Confirming the Northern blot analysis significant levels of TTYH2 mRNA was detected in the brain (nos. 1-21), heart (nos. 22-29) and testis (no. 54) (FIG. 5B). There was also low levels of expression of TTYH2 in all other tissues and cell lines on the blot.

Using DD-PCR, TTYH2 was shown to be up-regulated in 4 out of 4 paired samples. To confirm the up-regulation of this gene in RCC and to examine a larger number of samples, semi-quantitative RT-PCR was performed on a further 12 matched RCC and normal kidney paired samples (male and female). In this regard, total RNA was extracted from these patient's (6 male and 6 female) paired RCC and normal kidney tissue samples as well as from cell preparations (10⁶ cells) of 2 RCC cell lines, Caki 1 and SN12K1, using TRI-Reagent (Sigma) according to the manufacturer's instructions. For cDNA synthesis, 2 mg of total RNA was reverse transcribed using Superscript II (Life Technologies). RT-PCR was performed using the following primers 5′-GGTGAGGCCGCATGTATATAAGC-3′ and 5′-GGTATATCCGCGTCACATGCAG-3′. Optimum cycling parameters, shown to be in the linear range of amplification, were 94° C. for 1 min, 59° C. for 1 min and 72° C. for 1 min for 26 cycles followed by a further 7 mins extension at 72° C. A control PCR was also performed for β2-microglobulin for 25 cycles.

Up-regulation of TTYH2 in RCC was demonstrated in 9 out of the 12 (75%) paired samples (FIG. 5C) using the above RT-PCR procedure. In addition, RT-PCR performed on the renal cell carcinoma-derived cell lines, Caki 1 and SN12K1, showed that this gene is expressed in both these cell lines (FIG. 5C). The Caki I and SN12K1 cell lines were derived from RCC metastases to skin and lung respectively. Analysis of the source of the ESTs matching the TTYH2 sequence revealed that this gene is also expressed in other malignancies with many of the ESTs (28%) isolated from brain tumour libraries. Furthermore, analysis of the ESTs that match TTYH1 revealed that this gene may have a similar expression pattern to that of TTYH2. Of the 49 ESTs matching TTYH1, 25 (51%) were isolated from normal or malignant brain tissue. Interestingly, 10 (20%) ESTs matching TTYH1 were isolated from testicular-derived germ cell tumours.

In summary, the above data indicate that in normal tissues TTYH2 is expressed abundantly in brain and testis with lower levels of expression in heart and ovary, that TTYH2 expression is up-regulated in RCC, and that brain tumours and testicular-derived germ cell tumours express this gene. Interestingly, using comparative genomic hybridisation, high-level amplification of 17q24-q25, the region containing the TTYH2 gene, has been shown in adrenocortical tumours (Dohna et al., 2000), brain metastases of solid tumours (Petersen et al., 2000) and muscle invasive bladder cancer (Simon et al., 2000). Whether amplification of this genomic region is the mechanism of TTYH2 up-regulation in RCC is not yet known. However, if TTYH2 does function as a cell surface receptor, it is possible that its up-regulation may give a growth advantage or metastatic ability to cancer cells, particularly those of kidney, brain and testis origin.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

Tables

TABLE 1

PCR primers and annealing temperatures used to
amplify TTYH2 introns from BAC 2514K5

| Intron | | SEQ | | | SEQ |
|---|---|---|---|---|---|
| ampli- | Forward primer | ID | Reverse primer | Temp | ID |
| fied | 5′-3′ | NO. | 5′-3′ | (° C.) | NO. |
| A | CCGTGAACAGCACCTTCAGCCC | 13 | CCAGCCCCAGGAACAGCAGCG | 64 | 14 |
| B | CCTGCTGCATCACCTGGACG | 15 | AAACCAACGCCCACCGCAGC | 61 | 16 |
| C | CCACACCTTCTCTGGGATCG | 17 | CCAGGTGCTGCTCTAGGTCC | 66 | 18 |
| D | CCCGGCTCAGTGAGATCTTTGC | 19 | GAGCAGGAGGTAGGAGAGCC | 68 | 20 |
| F | CCTCAGTTGGGCATCCCTGG | 21 | AGCCACACAGAAGTCACTGG | 66 | 22 |
| K | CCTTCTCCACCATGATCTGTGC | 23 | CCACTGCTGTAGCTGCAGAAGC | 63 | 24 |
| L | CCTGTCTCCGAGTACATGAACC | 25 | CGTAGCGTGGGTTCCTACC | 60 | 26 |
| M | CACTAATCGGGAGAGCCTCC | 27 | CGTGGTGAGTCTTCTGCACCC | 60 | 28 |

### BIBLIOGRAPHY

Bentley S., and Bassam B. J. (1996). A robust DNA amplification fingerprint system applied to the analysis of genetic variation within Fusarium oxysporum f.sp. cubense. J Phytopath. 144: 207-213.
Callen D. F., Baker, E., Eyre, H. J., Chernos, J. E., Bell, J. A. and Sutherland, G. R. (1990). Reassessment of two apparent deletions of chromosome 16p to an ins(11;16) and a t(1;16) by chromosome painting. Ann Genet. 33: 219-221.
Campbell H. D., Kamei, M., Claudianos, C., Woollatt, E., Sutherland, G. R., Suzuki, Y., Hida, M., Sugano, S. and Young, I. G. (2000). Human and mouse homologues of the Drosophila melanogaster tweety (tty) gene: a novel gene family encoding predicted transmembrane proteins. Genomics 68: 89-92.
Chen K. S., Gunaratne, P. H., Hoheisel, J. D., Young, I. G., Miklos, G. L., Greenberg, F., Shaffer, L. G., Campbell, H. D. and Lupski, J. R. (1995). The human homologue of the Drosophila melanogaster flightless-I gene fli1) maps within the Smith-Magenis microdeletion critical region in 17p11.2. Am J Hum Genet. 56: 175-182.
Chen L. C., Manjeshwar S., Lu Y., Moore D., Ljung B. M., Kuo W. L., Dairkee S. H., Wernick M., Collins C., and Smith H. S. (1998). The human homologue for the Caenorhabditis elegans cul-4 gene is amplified and overexpressed in primary breast cancers. Cancer Res. 58: 3677-83.
Cole K. A., Chuaqui R. F., Katz K., Pack S., Zhuang Z., Cole C. E., Lyne J. C., Linehan W. M., Liotta L. A., and Emmert-Buck M. R. (1998). cDNA sequencing and analysis of POV1 (PB39): a novel gene up-regulated in prostate cancer. Genomics 51: 282-7.
Dohna M., Reincke, M., Mincheva, A., Allolio, B., Solinas-Tolda, S. and Lichter, P. (2000). Adrenocortical carcinoma is characterized by a high frequency of chromosomal gains and high-level amplifications. Genes Chromosomes Cancer 28: 145-152.
D'Souza S. E., Ginsberg, M. H. and Plow, E. F. (1991). Arginyl-glycyl-aspartic acid (RGD): a cell adhesion motif. Trends Biochem Sci. 16: 246-250. Fleming S. (1998). Genetics of kidney tumours. Forum (Genova) δ: 176-84.
Guinan P., Sobin, L. H., Algaba, F., Badellino, F., Kameyama, S., MacLennan, G., Novick, A. (1997). TNM staging of renal cell carcinoma: Workgroup No. 3. Union International Contre le Cancer (UICC) and the American Joint Committee on Cancer (AJCC). Cancer 80: 992-3.
Ivanov S. V., Kuzmin I., Wei M. H., Pack S., Geil L., Johnson B. E., Stanbridge E. J., and Lerman M. I. (1998). Down-regulation of transmembrane carbonic anhydrases in renal cell carcinoma cell lines by wild-type von Hippel-Lindau transgenes. Pro Natl Acad Sci USA. 95: 12596-601.
Kent J., Wheatly, S. C., Andrews, J. E., Sinclair, A. H. and Koopman, P. (1996). A male-specific role for SOX9 in vertebrate sex dertirmination. Development 122: 2813-2822. Kocher O., Cheresh P., Brown L. F., and Lee S. W. (1995). Identification of a novel gene, selectively up-regulated in human carcinomas, using the differential display technique. Clin Cancer Res. 1: 1209-15.
Kovacs G., Akhtar, M, Beckwith, B J, Bugert, P, Cooper, C S, Delahunt, B, Eble, J N, Fleming, S, Ljungberg, B, Medeiros, L J, Moch, H, Reuter, V E, Ritz, E, Roos, G,
Schmidt, D, Srigley, J R, Storkel, S, van den Berg, E, Zbar, B. (1997). The Heidelberg classification of renal cell tumours. J Pathol. 183: 131-3.
Kyte J., Doolittle, R. F. (1982). A simple method for displaying the hydropathic character of a protein. J Mol Biol. 157: 105-132.
Latif F., Tory, K., Gnarra, J., Yao, M., Duh, F. M., Orcutt, M. L., Stackhouse, T., Kuzmin, I., Modi, W., Geil, L. (1993). Identification of the von Hippel-Lindau disease tumor suppressor gene. Science 260: 1317-20.
Liang P., and Pardee A. B. (1992). Differential display of eukaryotic messenger RNA by means of the polymerase chain reaction. Science 257: 967-71.
Maher E. R., Bentley, E., Yates, J. R., Latif, F., Lerman, M., Zbar, B., Affara, N. A. and Ferguson-Smith, M. A. (1991). Mapping of the von Hippel-Lindau disease locus to a small region of chromosome 3p by genetic linkage analysis. Genomics 10: 957-60.
Maleszka R., De Couet, H. G. and Gabor Miklos, G. L. (1998). Data transferability from model organisms to human beings: Insights from the functional genomics of the flightless region of Drosophila. Proc Natl Acad. Sci. USA 95: 3731-3736.
Mok S. C., Chan W. Y., Wong K. K., Cheung K. K., Lau C. C., Ng S. W., Baldini A., Colitti C. V., Rock C. O., and Berkowitz R. S. (1998). DOC-2, a candidate tumor suppressor gene in human epithelial ovarian cancer. Oncogene 16: 2381-7.
Petersen I., Hidalgo, A., Petersen, S., Schluns, K., Schewe, C., Pacyna-Gengelbach, M., Goeze, A., Krebber, B., Knosel, T., Kaufmann, O., Szymas, J. and von Deimling, A. (2000). Chromosomal imbalances in brain metastases of solid tumours. Brain Pathol. 10: 395-401.
Rae F. K., Stephenson, S-A., Nicol, D. L. and Clements, J. A. (2000). Novel association of a diverse range of genes with renal cell carcinoma as identified by differential display. Int J Cancer 88: 726-732.
Sager R. (1997). Expression genetics in cancer: shifting the focus from DNA to RNA. Proc Natl Acad Sci USA. 94: 952-5.
Schmidt L., Duh, F. M., Chen, F., Kishida, T., Glenn, G., Choyke, P., Scherer, S. W., Zhuang, Z., Lubensky, I., Dean, M., Allikmets, R., Chidambaram, A., Bergerheim, U. R., Feltis, J. T., Casadevall, C., Zamarron, A., Bernues, M., Richard, S., Lips, C. J., Walther, M. M., Tsui, L. C., Geil, L., Orcutt, M. L., Stackhouse, T., Zbar, B. (1997). Germline and somatic mutations in the tyrosine kinase domain of the MET proto-oncogene in papillary renal carcinomas. Nat Genet. 16: 68-73.
Simon R., Burger H., Semjonow, A., Hertle, L., Terpe, H. J. and Bocker, W. (2000). Patterns of chromosomal imbalances in muscle invasive bladder cancer. Int J Oncol. 17: 1025-1029.
Stassar M. J., Pitzer, C. and Zoller, M. (1999). Downregulation of TNF receptor-associated protein-2/p97 in renal cell carcinoma. Oncology Res. 11: 85-90.
Teh B. T., Giraud, S., Sari, N. F., Hii, S. I., Bergerat, J. P., Larsson, C., Limacher, J. M. and Nicol, D. (1997). Familial non-VHL non-papillary clear-cell renal cancer. Lancet 349: 848-9.
Thrash-Bingham C. A., and Tartof K. D. (1999). aHIF: a natural antisense transcript overexpressed in human renal cancer and during hypoxia [see comments]. J Natl Cancer Inst. 91: 143-51.
Vita N., Laurent, P., Lefort, S., Chalon, P., Dumont, X., Kaghad, M., Gully, D., Le Fur, G., Ferrara, P. and Caput, D. (1993). Cloning and expression of a complementary DNA encoding a high affinity human neurotensin receptor. FEBS Lett. 317: 139-142.

1. An isolated polypeptide comprising an amino acid sequence corresponding to at least a biologically active fragment of the sequence set forth in SEQ ID NO: 2 or 7, or to a variant or derivative thereof.

2. The polypeptide of claim 1, wherein said biologically active fragment comprises at least 6 contiguous amino acids contained within the sequence set forth in any one of SEQ ID NO: 2 and 7.

3. The polypeptide of claim 2, wherein said biologically active fragment is selected from residues 1-57, 109-216 or 259-391 of SEQ ID NO: 2 or 7.

4. The polypeptide of claim 2, wherein said biologically active fragment is selected from residues 58-74, 92-108, 217-233, 240-258 or 392-408 of SEQ ID NO: 2 or 7.

5. The polypeptide of claim 2, wherein said biologically active fragment is selected from residues 75-91, 234-239, 409-534 of SEQ ID NO: 2, or residues 409-532 of SEQ ID NO: 7.

6. The polypeptide of claim 1, wherein said variant has at least 50% sequence identity to said at least a biologically active fragment.

7. The polypeptide of claim 6, wherein said variant is distinguished from at least a biologically active fragment of the sequence set forth in SEQ ID NO: 2 or 7 by the substitution of at least one amino acid residue.

8. The polypeptide of claim 6, wherein said variant is distinguished from at least a biologically active fragment of the sequence set forth in SEQ ID NO: 2 or 7 by the substitution of at least one amino acid residue, which is a conservative substitution.

9. An isolated polynucleotide comprising a nucleotide sequence encoding a polypeptide comprising an amino acid sequence corresponding to at least a biologically active fragment of the sequence set forth in SEQ ID NO: 2 or 7, or to a variant or derivative thereof.

10. An isolated polynucleotide comprising a nucleotide sequence that corresponds or is complementary to at least a portion of the sequence set forth in SEQ ID NO: 1, 3, 4, 6 or 8, or to a polynucleotide variant thereof.

11. The polynucleotide of claim 10, wherein said nucleotide sequence corresponds or is complementary to at least 18 contiguous nucleotides of the sequence set forth in SEQ ID NO: 1, 3, 4, 6 or 8.

12. The polynucleotide of claim 10, wherein said polynucleotide variant has at least 50% sequence identity to at least a portion of the sequence set forth in SEQ ID NO: 1, 3, 4, 6 or 8.

13. The polynucleotide of claim 10, wherein said variant is obtained from a mammal.

14. A vector comprising the polynucleotide of claim 10.

15. An expression vector comprising the polynucleotide of claim 10 in operable linkage with a regulatory polynucleotide.

16. A host cell containing the vector of claim 14 or the expression vector of claim 15.

17. A method of producing a recombinant polypeptide comprising an amino acid sequence corresponding to at least a biologically active fragment of the sequence set forth in SEQ ID NO: 2 or 7, or to a variant or derivative thereof, said method comprising:
culturing a host cell containing the expression vector of claim 15 such that said recombinant polypeptide is expressed from said polynucleotide; and
isolating the said recombinant polypeptide.

18. A method of producing a biologically active fragment of a polypeptide comprising the sequence set forth in SEQ ID NO: 2 or 7, comprising:
introducing a fragment of the polypeptide or a polynucleotide from which said fragment can be translated into a cell; and
detecting modulation of tumorigenesis, which indicates that said fragment is a biologically active fragment.

19. The method of claim 18, wherein said fragment is present in said cell at a level and/or functional activity that correlates with the presence or risk of a cancer or tumour.

20. he method of claim 19, wherein said level and/or functional activity corresponds to a level and/or functional activity of a polypeptide comprising the sequence set forth in SEQ ID NO: 2 or 7, which correlates with the presence or risk of said cancer or tumour.

21. The method of claim 18, wherein the cancer or tumour is associated with an organ selected from kidney, brain or testis.

22. The method of claim 18, wherein said cancer or tumour is a cancer or tumour of the kidney.

23. The method of claim 18, wherein said cancer or tumour is renal cell carcinoma.

24. A method of producing a polypeptide variant of a parent polypeptide comprising the sequence set forth in SEQ ID NO: 2 or 7, or a biologically active fragment thereof, said method comprising:
providing a modified polypeptide whose sequence is distinguished from the parent polypeptide by the substitution, deletion or addition of at least one amino acid;
introducing said modified polypeptide or a polynucleotide from which the modified polypeptide can be translated into a cell; and
detecting modulation of tumorigenesis, which indicates that said modified polypeptide is a polypeptide variant.

25. The method of claim 24, wherein said variant is present in said cell at a level and/or functional activity that correlates with the presence or risk of a cancer or tumour.

26. he method of claim 24, wherein said level and/or functional activity corresponds to a level and/or functional activity of a polypeptide comprising the sequence set forth in SEQ ID NO: 2 or 7, which correlates with the presence or risk of said cancer or tumour.

27. The method of claim 24, wherein the cancer or tumour is associated with an organ selected from kidney, brain or testis.

28. The method of claim 24, wherein the cancer or tumour is a cancer or tumour of the kidney.

29. The method of claim 24, wherein said cancer or tumour is renal cell carcinoma.

30. A method of producing a polypeptide variant of a parent polypeptide comprising the sequence set forth in any one of SEQ ID NO: 2 and 7, or a biologically active fragment thereof, said method comprising:
providing a modified polypeptide whose sequence is distinguished from the parent polypeptide or said biologically active fragment, by the substitution, deletion or addition of at least one amino acid;
contacting the modified polypeptide with an antigen-binding molecule that is immuno-interactive with said parent polypeptide or said biologically active fragment; and
detecting the presence of a complex comprising the antigen-binding molecule and the modified polypeptide, which indicates that said modified polypeptide is a variant.

31. A method of screening for an agent which modulates tumorigenesis, said method comprising:
contacting a preparation comprising:
(i) a polypeptide comprising an amino acid sequence corresponding to at least a biologically active fragment of the sequence set forth in SEQ ID NO: 2 or 7, or to a variant or derivative thereof; or
(ii) a polynucleotide comprising at least a portion of a genetic sequence that regulates said polypeptide, which is operably linked to a reporter gene, with a test agent; and
detecting a change in the level and/or functional activity of said polypeptide, or an expression product of said reporter gene, relative to a normal or reference level and/or functional activity in the absence of said test agent.

32. The method of claim 31, wherein inhibits or otherwise reduces tumorigenesis.

33. The method of claim 32, further characterised by detecting an a reduction in the level and/or functional activity of said polypeptide, or an expression product of said reporter gene, relative to said normal or reference level and/or functional activity.

34. (canceled)

35. An antigen-binding molecule that is immuno-interactive with a polypeptide comprising an amino acid sequence corresponding to at least a biologically active fragment of the sequence set forth in SEQ ID NO: 2 or 7, or to a variant or derivative thereof.

36. A method for detecting a specific polypeptide or polynucleotide sequence, comprising detecting a sequence of:
SEQ ID NO: 2, or a fragment thereof at least 6 amino acids in length; or
SEQ ID NO: 7, or a fragment thereof at least 6 amino acids in length; or
SEQ ID NO: 1, or a fragment thereof at least 18 nucleotides in length; or
SEQ ID NO: 3, or a fragment thereof at least 18 nucleotides in length; or
SEQ ID NO: 4, or a fragment thereof at least 18 nucleotides in length; or
SEQ ID NO: 6, or a fragment thereof at least 18 nucleotides in length; or
SEQ ID NO: 8, or a fragment thereof at least 18 nucleotides in length.

37. A method for detecting a polypeptide comprising an amino acid sequence corresponding to at least a biologically active fragment of the sequence set forth in SEQ ID NO: 2 or 7, or to a variant or derivative thereof, said method comprising:
detecting expression in a cell of a polynucleotide comprising a nucleotide sequence encoding said polypeptide.

38. A method of detecting a polypeptide comprising an amino acid sequence corresponding to at least a biologically active fragment of the sequence set forth in SEQ ID NO: 2 or 7, or to a variant or derivative thereof in a biological sample, said method comprising:
contacting the sample with an antigen-binding molecule that is immuno-interactive with said polypeptide; and
detecting the presence of a complex comprising said antigen-binding molecule and said polypeptide in said contacted sample.

39. A method for detecting the presence or diagnosing the risk of a cancer or tumour in a patient, comprising detecting aberrant expression of TTYH2 in a biological sample obtained from said patient.

40. The method of claim 39, wherein said aberrant expression is detected by detecting a level and/or functional activity of a TTYH2 expression product in said biological sample, which differs from a normal reference level and/or functional activity and which correlates with presence or risk of said cancer or tumour.

41. The method of claim 40, wherein said aberrant expression is detected by detecting a higher level and/or functional activity of said expression product than said normal reference level and/or functional activity.

42. The method of claim 40, wherein the level and/or functional activity of said expression product in said biological sample is at least 110% of that which is present in a corresponding biological sample obtained from a normal individual or from an individual who is not afflicted with said cancer or tumour.

43. The method of claim 39, wherein the cancer or tumour is associated with an organ selected from kidney, brain or testis.

44. The method of claim 39, wherein said cancer or tumour is a cancer or tumour of the kidney.

45. The method of claim 39, wherein said cancer or tumour is renal cell carcinoma.

46. A method for detecting the presence or diagnosing the risk of a cancer or tumour in a patient, comprising detecting in a biological sample obtained from said patient an aberrant level and/or functional activity of an expression product of a gene selected from TTYH2 or a gene relating to the same regulatory or biosynthetic pathway as TTYH2, wherein said aberrant level and/or functional activity correlates with the presence or risk of said cancer or tumour.

47. The method of claim 46, wherein said expression product is expressed at a higher level and/or functional activity than said normal reference level and/or functional activity.

48. The method of claim 46, wherein said aberrant level and/or functional activity is at least 110% of that which is present in a corresponding biological sample obtained from a normal individual or from an individual who is not afflicted with said cancer or tumour.

49. The method of claim 46, wherein the cancer or tumour is associated with an organ selected from kidney, brain or testis.

50. The method of claim 46, wherein said cancer or tumour is a cancer or tumour of the kidney.

51. The method of claim 46, wherein said cancer or tumour is renal cell carcinoma.

52. A method for diagnosing the progression of a cancer or tumour in a patient, comprising measuring aberrant TTYH2 expression in a biological sample obtained from said patient.

53. A method for prognostic assessment of a cancer or tumour in a patient, comprising detecting aberrant TTYH2 expression n a biological sample obtained from said patient.

54. A method for detecting the presence or diagnosing the risk of a cancer or tumour in a patient, comprising:
providing a biological sample from said patient; and
detecting relative to a normal reference value, an elevation in the level and/or functional activity of a member selected from the group consisting of a polypeptide comprising the sequence set forth in any one of SEQ ID NO: 2 and 7, or variant thereof, and a polynucleotide comprising the sequence set forth in any one of SEQ ID NO: 1, 3, 6 and 8, or variant thereof.

55. The method of claim 54, wherein said member is present in said biological sample at a higher level and/or functional activity than in a corresponding biological sample obtained from a normal individual or from an individual who is not afflicted with said cancer or tumour.

56. The method of claim 55, wherein said higher level and/or functional activity is at least 110% of that which is present in said corresponding biological sample.

57. The method of claim 54, wherein the cancer or tumour is associated with an organ selected from kidney, brain or testis.

58. The method of claim 54, wherein said cancer or tumour is a cancer or tumour of the kidney.

59. The method of claim 54, wherein said cancer or tumour is renal cell carcinoma.

60. A method for detecting the presence or diagnosing the risk of a cancer or tumour in a patient, comprising:
providing a biological sample from said patient; and
detecting aberrant expression of a TTYH2 polynucleotide or a TTYH2 polypeptide.

61. The method of claim 60, wherein said TTYH2 polynucleotide or said TTYH2 polypeptide is present in said biological sample at a higher level and/or functional activity than in a corresponding biological sample obtained from a normal individual or from an individual who is not afflicted with said cancer or tumour.

62. The method of claim 61, wherein said higher level and/or functional activity is at least 110% of that which is present in said corresponding biological sample.

63. The method of claim 60, wherein the cancer or tumour is associated with an organ selected from kidney, brain or testis.

64. The method of claim 60, wherein said cancer or tumour is a cancer or tumour of the kidney.

65. The method of claim 60, wherein said cancer or tumour is renal cell carcinoma.

66. A method for detecting the presence or diagnosing the risk of a cancer or tumour in a patient, comprising:
contacting a biological sample obtained from said patient with an antigen-binding molecule that is immuno-interactive with a polypeptide comprising an amino acid sequence corresponding to at least a biologically active fragment of the sequence set forth in SEQ ID NO: 2 or 7, or to a variant or derivative thereof,
measuring the concentration of a complex comprising said antigen-binding molecule and a polypeptide comprising the sequence set forth in SEQ ID NO: 2 or 7, or a variant thereof, in said contacted sample; and
relating said measured complex concentration to the concentration of said polypeptide in said sample, wherein the presence of an elevated concentration relative to a normal reference concentration is indicative of said cancer or tumour.

67. The method of claim 66, wherein said concentration of said polypeptide in said sample is at least 110% of that which is present in a corresponding biological sample obtained from a normal individual or from an individual who is not afflicted with said cancer or tumour.

68. The method of claim 67, wherein the cancer or tumour is associated with an organ selected from kidney, brain or testis.

69. The method of claim 67, wherein said cancer or tumour is a cancer or tumour of the kidney.

70. The method of claim 67, wherein said cancer or tumour is renal cell carcinoma.

71. A method for modulating tumorigenesis, said method comprising introducing into said cell an agent for a time and under conditions sufficient to modulate the level and/or functional activity of TTYH2 wherein said agent is identifiable by a screening method comprising:
contacting a preparation comprising:
(i) a polypeptide comprising an amino acid sequence corresponding to at least a biologically active fragment of the sequence set forth in SEQ ID NO: 2 or 7, or to a variant or derivative thereof; or
(ii) a polynucleotide comprising at least a portion of a genetic sequence that regulates said polypeptide, which is operably linked to a reporter gene, with a test agent; and
detecting a change in the level and/or functional activity of said polypeptide, or an expression product of said reporter gene, relative to a normal or reference level and/or functional activity in the absence of said test agent.

72. The method of claim 71, wherein said agent decreases the level and/or functional activity of TTYH2.

73. The method of claim 71, wherein said agent is an antisense oligonucleotide or ribozyme that binds to, or otherwise interacts specifically with, a polynucleotide encoding TTYH2 or complement of thereof, or variant of these.

74. The method of claim 71, wherein said agent is an antigen-binding molecule that is immuno-interactive with TTYH2 or variant thereof.

75. A composition for delaying, repressing or otherwise inhibiting tumorigenesis, comprising an agent that reduces the level and/or functional activity of TTYH2, and optionally a pharmaceutically acceptable carrier.

76. A composition for treatment and/or prophylaxis of a cancer or tumour, comprising an agent that reduces the level and/or functional activity of TTYH2, an optionally a pharmaceutically acceptable carrier.

77. A method for treatment and/or prophylaxis of a cancer or tumour, said method comprising administering to a patient in need of such treatment an effective amount of an agent that reduces the level and/or functional activity of TTYH2, and optionally a pharmaceutically acceptable carrier wherein said agent is identifiable by a screening method comprising:
contacting a preparation comprising:
(i) a polypeptide comprising an amino acid sequence corresponding to at least a biologically active fragment of the sequence set forth in SEQ ID NO: 2 or 7, or to a variant or derivative thereof; or
(ii) a polynucleotide comprising at least a portion of a genetic sequence that regulates said polypeptide, which is operably linked to a reporter gene, with said agent; and
detecting inhibition or reduction in the level and/or functional activity of said polypeptide, or an expression product of said reporter gene, relative to a normal or reference level and/or functional activity in the absence of said agent.

78. (canceled)

79. (canceled)

80. A non-human genetically modified animal model for TTYH2 function, wherein the genetically modified animal is characterised by having an altered TTYH2 gene.

81. The genetically modified animal of claim 80, comprising an alteration to its genome, wherein the alteration comprises replacement of an endogenous TTYH2 gene with a foreign TTYH2 gene.

82. The genetically modified animal of claim 80, comprising an alteration to its genome, wherein the alteration corresponds to a partial or complete loss of function in one or both alleles of the endogenous TTYH2 gene.

83. The genetically modified animal of claim 80, comprising a disruption in at least one allele of the endogenous TTYH2 gene.

84. A composition, comprising an immunopotentiating agent selected from the polypeptide of claim 1, or the polynucleotide of claim 10, or the vector of claim 14 or the expression vector of claim 15, together with a pharmaceutically acceptable carrier.

85. The composition of claim 84, further comprising an adjuvant.

86. A method for modulating an immune response against a cancer or tumour, comprising administering to a patient in need of such treatment an effective amount of an immunopotentiating agent selected from the polypeptide of claim 1, or the polynucleotide of claim 10, or the vector of claim 14 or the expression vector of claim 15.

87. The method of claim 86, wherein the cancer or tumour is associated with an organ selected from kidney, brain or testis.

88. The method of claim 86, wherein said cancer or tumour is a cancer or tumour of the kidney.

89. The method of claim 86, wherein said cancer or tumour is renal cell carcinoma.
